(19) United States
(12) Patent Application Publication
(10) Pub. No.: US 2025/0186586 A1
(43) Pub. Date: Jun. 12, 2025

(54) METHODS OF ISOLATING AND ENHANCING POPULATIONS OF TUMOR-REACTIVE LYMPHOCYTES

(71) Applicant: SKDW HOLDINGS, LLC, Glencoe, IL (US)

(72) Inventors: Shana KELLEY, Glencoe, IL (US); Zongjie WANG, Chicago, IL (US)

(21) Appl. No.: 18/954,020

(22) Filed: Nov. 20, 2024

Related U.S. Application Data
(63) Continuation of application No. PCT/US2023/023070, filed on May 22, 2023.
(60) Provisional application No. 63/344,608, filed on May 22, 2022.
(60) Provisional application No. 63/358,513, filed on Jul. 5, 2022.
(60) Provisional application No. 63/390,195, filed on Jul. 18, 2022.
(60) Provisional application No. 63/491,256, filed on Mar. 20, 2023.

(51) Int. Cl. A61K 40/11; A61K 40/22; A61K 40/42; A61P 35/00; C12M 1/12; C12M 3/06; C12N 5/0783

(52) U.S. Cl. CPC A61K 40/11; A61K 40/22; A61K 40/428; A61P 35/00; C12M 23/16; C12M 25/16; C12N 5/0636; C12N 2502/30

(57) ABSTRACT

Provided herein are methods of isolating and expanding a population of tumor reactive lymphocytes (TRLs) from a fluid sample of a subject in need thereof. In some embodiments, methods of isolating and expanding a population of TRLs comprise flowing the TRLs across a magnetic capture zone of a microfluidic device. Also provided herein are methods of administering a population of TRLs to a subject in need thereof. Also provided herein are compositions comprising therapeutically enhanced TRLs.

### CROSS-REFERENCE

This application is a continuation application of International Application No. PCT/US2023/023070, filed May 22, 2023, which claims the benefit of U.S. Provisional Application No. 63/491,256, filed Mar. 20, 2023, U.S. Provisional Application No. 63/390,195, filed Jul. 18, 2022, U.S. Provisional Application No. 63/358,513, filed Jul. 5, 2022, and U.S. Provisional Application No. 63/344,608, filed May 22, 2022, each of which is hereby incorporated by reference in its entirety.

### BACKGROUND

Adoptive cellular therapy (ACT) is a form of immunotherapy that uses cells from patient's immune systems, such as T cells, as a treatment for cancer. ACT involves isolating and modifying patient's immune cells, and then reinfusing the immune cells into patient to enhance its ability to fight cancer.

### SUMMARY

Described herein is a method of enriching a population of tumor-reactive lymphocytes (TRLs), the method comprising: enriching from a peripheral blood sample of a subject a population of lymphocytes comprising CD103. In some embodiments, the enriching comprises magnetically separating by flowing the population of lymphocytes across a magnetic capture zone disposed in a channel of a microfluidic device. In some embodiments, the fluid sample is a peripheral blood sample. In some embodiments, the fluid sample comprises a pleural effusion. In some embodiments, the fluid sample comprises ascites. In some embodiments, the magnetically separating achieves a recovery of at least 80%. In some embodiments, the magnetically separating achieves a recovery of at least 90%. In some embodiments, the purity of isolation of the population of TRLs is at least 60%. In some embodiments, the purity of isolation of the population of TRLs is at least 70%. In some embodiments, the purity of isolation of the population of TRLs is at least 80%. In some embodiments, the magnetically separating comprises contacting the fluid sample with an antibody capable of binding to a TRL surface protein. In some embodiments, the TRL surface protein is CD3, CD4, CD8, CD39, CD103, SLC6A19, SIDT1 or any combination thereof. In some embodiments, the TRL surface protein is CD103. In some embodiments, the TRL surface protein is CD39. In some embodiments, the TRL surface protein is SLC6A19. In some embodiments, the TRL surface protein is SIDT1. In some embodiments, the antibody is conjugated to a magnetic nanoparticle. In some embodiments, the magnetically separating comprises contacting the fluid sample with a second antibody capable of binding to the antibody capable of binding to a TRL surface protein. In some embodiments, the second antibody is conjugated to a magnetic nanoparticle. In some embodiments, the magnetically separating comprises contacting the fluid sample with a plurality of major histocompatibility complex (MHC) multimers mimicking a tumor epitope, herein the fluid sample comprises a peripheral blood sample. In some embodiments, at least one of the plurality of MHC multimers is conjugated to a linker molecule. In some embodiments, the linker molecule comprises a fluorophore. In some embodiments, the linker molecule is conjugated to a magnetic particle. In some embodiments, the magnetic particle comprises a magnetic nanoparticle. In some embodiments, the device comprises a plurality of magnetic capture zones, wherein the plurality of magnetic capture zones are disposed to spatially separate cells with different degrees of magnetization. In some embodiments, the subject has a cancer or is suspected of having a cancer. In some embodiments, the cancer is in a tissue, and wherein the tissue comprises a breast tissue, a renal tissue, a cervical tissue, a lung tissue, an ovarian tissue, or a skin tissue. In some embodiments, the method further comprises culturing the magnetically separated TRLs. In some embodiments, the method further comprises introducing a cell from the cultured TRLs to a nucleotide that expresses a therapeutically enhancing polypeptide under conditions sufficient to produce the enhancing polypeptide by the cell. In some embodiments, the method further comprises culturing the cell to produce a therapeutically enhanced cell. In some embodiments, the nucleotide encodes a chimeric antigen receptor (CAR). In some embodiments, the CAR comprises an antigen binding domain. In some embodiments, the antigen binding domain binds to a tumor antigen. In some embodiments, the nucleotide encodes an engineered T-Cell receptor. In some embodiments, the engineered T-Cell receptor comprises an antigen binding domain. In some embodiments, the antigen binding domain binds to a tumor antigen. In some embodiments, the method further comprises administering to the subject a therapeutically effective amount of the cultured TRL. In some embodiments, the subject is treated for a cancer or pre-cancer. In some embodiments, the cancer is in a tissue, and wherein the tissue comprises a breast tissue, a renal tissue, a cervical tissue, an ovarian tissue, a lung tissue, or a skin tissue. In some embodiments, the therapeutically enhanced cells are administered together with an additional cancer therapy. In some embodiments, the additional cancer therapy comprises an immune checkpoint inhibitor therapy. In some embodiments, the additional cancer therapy comprises a costimulatory therapy. In some embodiments, the costimulatory therapy comprises administering a GITR. In some embodiments, the additional cancer therapy is an anti-PD1 or anti-PD-L1 therapy. In some embodiments, the anti-PD1 or anti-PD-L1 therapy comprises an anti-PD1 antibody or antigen-binding fragment thereof. In some embodiments, the population of tumor-reactive lymphocytes (TRLs) comprises a population of circulating tumor-reactive lymphocytes (CTRLs).

Described herein is a method of enriching a population of tumor-reactive lymphocytes (TRLs), the method comprising: enriching from a fluid sample of a subject a population of lymphocytes comprising SLC6A19, SIDT1, or SLC6A19 and SIDT1. In some embodiments, the population of lymphocytes further comprises CD3, CD4, CD8, CD39, CD103, or any combination thereof. In some embodiments, the population of lymphocytes comprise CD8, CD103, SLC6A19, and SIDT1. In some embodiments, the enriching comprises magnetically separating by flowing the population of lymphocytes across a magnetic capture zone disposed in a channel of a microfluidic device. In some embodiments, the fluid sample is a peripheral blood sample. In some embodiments, the fluid sample comprises a pleural effusion. In some embodiments, the fluid sample comprises ascites. In some embodiments, the magnetically separating achieves a recovery of at least 70%. In some embodiments, the magnetically separating achieves a recovery of at least 80%. In some embodiments, the magnetically separating achieves a recovery of at least 90%. In some embodiments, the purity of isolation of the population of TRLs is at least 60%. In some embodiments, the purity of isolation of the population of TRLs is at least 70%. In some embodiments, the purity of isolation of the population of TRLs is at least 80%. In some embodiments, the magnetically separating comprises contacting the fluid sample with an antibody capable of binding to a TRL surface protein. In some embodiments, the TRL surface protein is CD3, CD4, CD8, CD39, CD103, SLC6A19, SIDT1 or any combination thereof. In some embodiments, the TRL surface protein is CD103. In some embodiments, the TRL surface protein is CD39. In some embodiments, the TRL surface protein is SLC6A19. In some embodiments, the TRL surface protein is SIDT1. In some embodiments, the antibody is conjugated to a magnetic nanoparticle. In some embodiments, the magnetically separating comprises contacting the fluid sample with a second antibody capable of binding to the antibody capable of binding to a TRL surface protein. In some embodiments, the second antibody is conjugated to a magnetic nanoparticle. In some embodiments, the magnetically separating comprises contacting the fluid sample with a plurality of major histocompatibility complex (MHC) multimers mimicking a tumor epitope, herein the fluid sample comprises a peripheral blood sample. In some embodiments, at least one of the plurality of MHC multimers is conjugated to a linker molecule. In some embodiments, the linker molecule comprises a fluorophore. In some embodiments, the linker molecule is conjugated to a magnetic particle. In some embodiments, the magnetic particle comprises a magnetic nanoparticle. In some embodiments, the device comprises a plurality of magnetic capture zones, wherein the plurality of magnetic capture zones are disposed to spatially separate cells with different degrees of magnetization. In some embodiments, the subject has a cancer or is suspected of having a cancer. In some embodiments, the cancer is in a tissue, and wherein the tissue comprises a breast tissue, a renal tissue, a cervical tissue, a lung tissue, an ovarian tissue, or a skin tissue. In some embodiments, the method further comprises culturing the magnetically separated TRLs. In some embodiments, the method further comprises introducing a cell from the cultured TRLs to a nucleotide that expresses a therapeutically enhancing polypeptide under conditions sufficient to produce the enhancing polypeptide by the cell. In some embodiments, the method further comprises culturing the cell to produce a therapeutically enhanced cell. In some embodiments, the nucleotide encodes a chimeric antigen receptor (CAR). In some embodiments, the CAR comprises an antigen binding domain. In some embodiments, the antigen binding domain binds to a tumor antigen. In some embodiments, the nucleotide encodes an engineered T-Cell receptor. In some embodiments, the engineered T-Cell receptor comprises an antigen binding domain. In some embodiments, the antigen binding domain binds to a tumor antigen. In some embodiments, the method further comprises administering to the subject a therapeutically effective amount of the cultured TRL. In some embodiments, the subject is treated for a cancer or pre-cancer. In some embodiments, the cancer is in a tissue, and wherein the tissue comprises a breast tissue, a renal tissue, a cervical tissue, an ovarian tissue, a lung tissue, or a skin tissue. In some embodiments, the therapeutically enhanced cells are administered together with an additional cancer therapy. In some embodiments, the additional cancer therapy comprises an immune checkpoint inhibitor therapy. In some embodiments, the additional cancer therapy comprises a costimulatory therapy. In some embodiments, the costimulatory therapy comprises administering a GITR. In some embodiments, the additional cancer therapy is an anti-PD1 or anti-PD-L1 therapy. In some embodiments, the anti-PD1 or anti-PD-L1 therapy comprises an anti-PD1 antibody or antigen-binding fragment thereof. In some embodiments, the population of tumor-reactive lymphocytes (TRLs) comprises a population of circulating tumor-reactive lymphocytes (CTRLs).

Described herein is a method of isolating a population of tumor-reactive lymphocytes (TRLs), the method comprising magnetically separating from a fluid sample of a subject a population of CD103+ lymphocytes, wherein the magnetically separating comprises flowing the CD103+ lymphocytes across a magnetic capture zone disposed in a channel of a microfluidic device. In some embodiments, the fluid sample is a peripheral blood sample. In some embodiments, the fluid sample comprises a pleural effusion. In some embodiments, the fluid sample comprises ascites. In some embodiments, the CD103+ lymphocytes are CD8+CD103+ lymphocytes, CD3+CD103+ lymphocytes, CD4+CD103+ lymphocytes or CD39+CD103+ lymphocytes. In some embodiments, the CD103+ lymphocytes comprise CD8+CD103+SLC6A19+ lymphocytes, CD3+CD103+SLC6A19+ lymphocytes, or CD4+CD103+SLC6A19+ lymphocytes. In some embodiments, the CD103+ lymphocytes comprise CD8+CD103+SLC6A19+SIDT1+ lymphocytes, CD3+CD103+SLC6A19+SIDT1+ lymphocytes, CD4+CD103+SLC6A19+SIDT1+ lymphocytes. In some embodiments, the magnetically separating achieves a recovery of at least 70%. In some embodiments, the magnetically separating achieves a recovery of at least 80%. In some embodiments, the magnetically separating achieves a recovery of at least 90%. In some embodiments, the purity of isolation of the population of TRLs is at least 60%. In some embodiments, the purity of isolation of the population of TRLs is at least 70%. In some embodiments, the purity of isolation of the population of TRLs is at least 80%. In some embodiments, the magnetically separating comprises contacting the fluid sample with an antibody capable of binding to a TRL surface protein. In some embodiments, the TRL surface protein is CD3, CD4, CD8, CD39, CD103, SLC6A19, SIDT1 or any combination thereof. In some embodiments, the TRL surface protein is CD103. In some embodiments, the TRL surface protein is CD39. In some embodiments, the TRL surface protein is SLC6A19. In some embodiments, the TRL surface protein is SIDT1. In some embodiments, the antibody is conjugated to a magnetic nanoparticle. In some embodiments, the magnetically separating comprises contacting the fluid sample with a second antibody capable of binding to the antibody capable of binding to a TRL surface protein. In some embodiments, the second antibody is conjugated to a magnetic nanoparticle. In some embodiments, the magnetically separating comprises contacting the fluid sample with a plurality of major histocompatibility complex (MHC) multimers mimicking a tumor epitope, herein the fluid sample comprises a peripheral blood sample. In some embodiments, at least one of the plurality of MHC multimers is conjugated to a linker molecule. In some embodiments, the linker molecule comprises a fluorophore. In some embodiments, the linker molecule is conjugated to a magnetic particle. In some embodiments, the magnetic particle comprises a magnetic nanoparticle. In some embodiments, the device comprises a plurality of magnetic capture zones, wherein the plurality of magnetic capture zones are disposed to spatially separate cells with different degrees of magnetization. In some embodiments, the subject has a cancer or is suspected of having a cancer. In some embodiments, the cancer is in a tissue, and wherein the tissue comprises a breast tissue, a renal tissue, a cervical tissue, a lung tissue, an ovarian tissue, or a skin tissue. In some embodiments, the method further comprises culturing the magnetically separated TRLs. In some embodiments, the method further comprises introducing a cell from the cultured TRLs to a nucleotide that expresses a therapeutically enhancing polypeptide under conditions sufficient to produce the enhancing polypeptide by the cell. In some embodiments, the method further comprises culturing the cell to produce a therapeutically enhanced cell. In some embodiments, the nucleotide encodes a chimeric antigen receptor (CAR). In some embodiments, the CAR comprises an antigen binding domain. In some embodiments, the antigen binding domain binds to a tumor antigen. In some embodiments, the nucleotide encodes an engineered T-Cell receptor. In some embodiments, the engineered T-Cell receptor comprises an antigen binding domain. In some embodiments, the antigen binding domain binds to a tumor antigen. In some embodiments, the method further comprises administering to the subject a therapeutically effective amount of the cultured TRL. In some embodiments, the subject is treated for a cancer or pre-cancer. In some embodiments, the cancer is in a tissue, and wherein the tissue comprises a breast tissue, a renal tissue, a cervical tissue, an ovarian tissue, a lung tissue, or a skin tissue. In some embodiments, the therapeutically enhanced cells are administered together with an additional cancer therapy. In some embodiments, the additional cancer therapy comprises an immune checkpoint inhibitor therapy. In some embodiments, the additional cancer therapy comprises a costimulatory therapy. In some embodiments, the costimulatory therapy comprises administering a GITR. In some embodiments, the additional cancer therapy is an anti-PD1 or anti-PD-L1 therapy. In some embodiments, the anti-PD1 or anti-PD-L1 therapy comprises an anti-PD1 antibody or antigen-binding fragment thereof. In some embodiments, the population of tumor-reactive lymphocytes (TRLs) comprises a population of circulating tumor-reactive lymphocytes (CTRLs).

Described herein is a pharmaceutical formulation, comprising: a plurality of cells comprising: (i) a population of SLC6A19+ lymphocytes, (ii) a population of SIDT1+ lymphocytes; (iii) a population of CD103+ lymphocytes; or (iv) a combination of (i) to (iii); and a pharmaceutically acceptable: excipient, diluent, or vehicle, wherein the formulation is formulated for administration to a subject having or suspected of having cancer. In some embodiments, the formulation is formulated for administration with an additional therapeutic agent for the treatment of a cancer. In some embodiments, the additional therapeutic agent comprises an anti-PD1 antibody. In some embodiments, the CD103+ lymphocytes are CD8+CD103+ lymphocytes, CD3+CD103+ lymphocytes, CD4+CD103+ lymphocytes, or CD39+CD103+ lymphocytes. In some embodiments, the CD103+ lymphocytes are CD8+CD103+SLC6A19+ lymphocytes, CD3+CD103+SLC6A19+ lymphocytes, or CD4+CD103+SLC6A19+ lymphocytes. In some embodiments, the CD103+ lymphocytes are CD8+CD103+SLC6A19+SIDT1+ lymphocytes, CD3+CD103+SLC6A19+SIDT1+ lymphocytes, or CD4+CD103+SLC6A19+SIDT1+ lymphocytes. In some embodiments, the cancer is in a tissue of the subject, and wherein the tissue comprises a breast tissue, a renal tissue, a cervical tissue, a lung tissue, an ovarian tissue, or a skin tissue. In some embodiments, at least a portion of the plurality of cells further comprises a chimeric antigen receptor (CAR). In some embodiments, the CAR comprises an antigen binding domain. In some embodiments, the antigen binding domain binds to a tumor antigen. In some embodiments, at least a portion of the plurality of cells further comprises an engineered T-Cell receptor. In some embodiments, the engineered T-Cell receptor comprises an antigen binding domain. In some embodiments, the antigen binding domain binds to a tumor antigen.

Provided herein is an engineered tumor-reactive lymphocyte (TRL), comprising: (a) a lymphocyte comprising a cell surface marker, wherein the cell surface marker comprises CD3, CD4, CD39, CD103, SLC6A19, or SIDT1, or any combination thereof; and (b) a chimeric antigen receptor (CAR). In some embodiments, the lymphocyte comprises (i) CD8, CD39 and CD103; (ii) CD3, CD39 and CD103; (iii) CD4, CD39 and CD103; (iv) CD8, CD103, SLC6A19, and SIDT1; (v) CD3, CD103, SLC6A19, and SIDT1; or (vi) CD4, CD103, SLC6A19, and SIDT1. In some embodiments, the CAR comprises an antigen binding domain. In some embodiments, the antigen binding domain binds to a tumor antigen. In some embodiments, the engineered TRL disclosed herein is for use in treating cancer in a subject in need thereof.

Provided herein is a method of treating cancer in a subject, the method comprising administering to the subject cells that were produced by any of the methods disclosed herein. In some embodiments, the cancer is in a tissue of the subject, and wherein the tissue comprises a breast tissue, a renal tissue, a cervical tissue, a lung tissue, an ovarian tissue, or a skin tissue. In some embodiments, the method further comprises administering to the subject an additional cancer therapy. In some embodiments, the additional cancer therapy comprises an immune checkpoint inhibitor therapy. In some embodiments, the additional cancer therapy comprises a costimulatory therapy. In some embodiments, the costimulatory therapy comprises administering a GITR. In some embodiments, the additional cancer therapy is an anti-PD1 or anti-PD-L1 therapy. In some embodiments, the anti-PD1 or anti-PD-L1 therapy comprises an anti-PD1 antibody or antigen-binding fragment thereof.

Provided herein is a method of treating cancer in a subject, the method comprising administering to the subject the pharmaceutical formulation disclosed herein or the engineered TRL disclosed herein. In some embodiments, the cancer is in a tissue of the subject, and wherein the tissue comprises a breast tissue, a renal tissue, a cervical tissue, a lung tissue, an ovarian tissue, or a skin tissue. In some embodiments, the method further comprises administering to the subject an additional cancer therapy. In some embodiments, the additional cancer therapy comprises an immune checkpoint inhibitor therapy. In some embodiments, the additional cancer therapy comprises a costimulatory therapy. In some embodiments, the costimulatory therapy comprises administering a GITR. In some embodiments, the additional cancer therapy is an anti-PD1 or anti-PD-L1 therapy. In some embodiments, the anti-PD1 or anti-PD-L1 therapy comprises an anti-PD1 antibody or antigen-binding fragment thereof.

Provided herein is a kit, comprising: (a) the engineered TRL disclosed herein, or a pharmaceutical formulation disclosed herein; and (b) instructions for administering the engineered TRL to a subject in need thereof.

Provided herein is a kit, comprising: (a) cells that were produced by any of the methods disclosed herein; and (b) instructions for administering the cells to the subject.

Provided herein is a system, comprising: a fluid sample obtained from a subject, wherein the fluid sample comprises a population of cells comprising (i) CD103+ lymphocytes, (ii) SLC6A19+ lymphocytes, (iii) SIDT1+ lymphocytes, or (iv) any combination of (i) to (iii); and a microfluidic device comprising a magnetic capture zone disposed in a channel of the microfluidic device, wherein the microfluidic device is configured to magnetically separate the population of cells from the fluid sample. In some embodiments, the population of SLC6A19+ lymphocytes comprise CD103+SLC6A19+SIDT1+ lymphocytes. In some embodiments, the population of SLC6A19+ lymphocytes comprise CD8+CD103+SLC6A19+SIDT1+ lymphocytes. In some embodiments, the CD103+ lymphocytes are CD8+CD103+ lymphocytes. In some embodiments, the CD103+ lymphocytes are CD8+CD39+CD103+ lymphocytes. In some embodiments, the CD103+ lymphocytes comprise CD8+CD103+SLC6A19+ lymphocytes. In some embodiments, the fluid sample is a peripheral blood sample. In some embodiments, the fluid sample comprises a pleural effusion. In some embodiments, the fluid sample comprises ascites. In some embodiments, the system further comprises an antibody or antigen-binding fragment thereof capable of binding a TRL surface protein. In some embodiments, the TRL surface protein is CD8, CD103, SLC6A19, SIDT1 or a combination thereof. In some embodiments, the antibody or antigen-binding fragment is conjugated to a magnetic nanoparticle. In some embodiments, the system further comprises a plurality of major histocompatibility complex (MHC) multimers mimicking a tumor epitope. In some embodiments, at least one of the plurality of MHC multimers is conjugated to a linker molecule comprising a fluorophore or a magnetic particle. In some embodiments, the microfluidic device comprises a plurality of magnetic capture zones, wherein the plurality of magnetic capture zones are disposed to spatially separate cells with different degrees of magnetization. In some embodiments, the system further comprises a cell culture configured to culture therapeutically enhanced cells derived from the population of (i) the CD103+ lymphocytes, (ii) the SLC6A19+ lymphocytes, or (iii) the SIDT1+ lymphocytes. In some embodiments, the system further comprises a nucleic acid molecule encoding a chimeric antigen receptor (CAR), or an engineered T-Cell receptor (TCR). In some embodiments, the CAR or the engineered TCR comprise an antigen binding domain that binds to a tumor antigen. In some embodiments, the system further comprises a pharmaceutically acceptable: excipient, diluent, or vehicle, wherein the population of (i) the CD103+ lymphocytes, (ii) the SLC6A19+ lymphocytes, or (iii) the SIDT1+ lymphocytes is formulated with the pharmaceutically acceptable: excipient, diluent, or vehicle for administration to a subject having or suspected of having cancer. In some embodiments, the system further comprises an additional cancer therapy. In some embodiments, the additional cancer therapy comprises an immune checkpoint inhibitor therapy or a costimulatory therapy. In some embodiments, the additional cancer therapy is an anti-PD1 or anti-PD-L1 therapy. In some embodiments, the anti-PD1 or anti-PD-L1 therapy comprises an anti-PD1 antibody or antigen-binding fragment thereof.

Provided herein is a composition comprising cells derived from a sample enriched for lymphocytes comprising SLC6A19, SIDT1, CD103, or any combination of thereof. In some embodiments, the sample is enriched for the lymphocytes by a process comprising: magnetically separating the lymphocytes from the sample, wherein the magnetically separating comprises flowing the lymphocytes across a magnetic capture zone disposed in a channel of a microfluidic device. In some embodiments, the process results in the sample comprising a purity that is greater than or equal to about 70%. In some embodiments, the process results in the sample comprising a purity that is greater than or equal to about 80%. In some embodiments, the lymphocytes further comprise CD3, CD4, CD8, CD39, CD103, or any combination thereof. In some embodiments, the lymphocytes further comprise CD8, CD103, SLC6A19, and SIDT1. In some embodiments, the sample is a peripheral blood sample. In some embodiments, the lymphocytes further comprise (i) CD8 and CD103, (ii) CD3 and CD103, (iii) CD4 and CD103, (iv) CD39 and CD103. In some embodiments, the lymphocytes further comprise (i) CD8, CD103, and SLC6A19, (ii) CD3, CD103, and SLC6A19, or (iv) CD4, CD103, and SLC6A19. In some embodiments, the lymphocytes further comprise (i) CD8, CD103, SLC6A19, and SIDT1, (ii) CD3, CD103, SLC6A19, and SIDT1, or (iii) CD4, CD103, SLC6A19, and SIDT1. In some embodiments, the lymphocytes comprise at least about 20,000 CD103+ cells per 10 million peripheral blood mononuclear cells. In some embodiments, the lymphocytes comprises at least about 2,000 CD103+ cells per 10 million peripheral blood mononuclear cells. In some embodiments, the lymphocytes comprise at least about 20,000 CD103+ cells, wherein the CD103+ cells comprising the purity that is greater than or equal to about 80%. In some embodiments, the lymphocytes comprise at least about 2,000 CD103+ cells, wherein the CD103+ cells comprising the purity that is greater than or equal to about 80%. In some embodiments, the sample is enriched with the lymphocytes by greater than or equal to about 70%. In some embodiments, the sample is enriched with the lymphocytes by greater than or equal to about 80%. In some embodiments, the sample has a purity of the lymphocytes that is greater than or equal to about 70%. In some embodiments, the sample has a purity of the lymphocytes that is greater than or equal to about 80%.

Provided herein is a composition comprising cells derived from a sample enriched for lymphocytes comprising SLC6A19, SIDT1, CD103, or any combination of thereof, wherein the sample is enriched for the lymphocytes by a process comprising the method disclosed herein. In some embodiments, the lymphocytes further comprise CD3, CD4, CD8, CD39, CD103, or any combination thereof. In some embodiments, the lymphocytes further comprise CD8, CD103, SLC6A19, and SIDT1. In some embodiments, the sample is a peripheral blood sample. In some embodiments, the lymphocytes further comprise (i) CD8 and CD103, (ii) CD3 and CD103, (iii) CD4 and CD103, (iv) CD39 and CD103. In some embodiments, the lymphocytes further comprise (i) CD8, CD103, and SLC6A19, (ii) CD3, CD103, and SLC6A19, or (iv) CD4, CD103, and SLC6A19. In some embodiments, the lymphocytes further comprise (i) CD8, CD103, SLC6A19, and SIDT1, (ii) CD3, CD103, SLC6A19, and SIDT1, or (iii) CD4, CD103, SLC6A19, and SIDT1. In some embodiments, the lymphocytes comprise at least about 20,000 CD103+ cells per 10 million peripheral blood mononuclear cells. In some embodiments, the lymphocytes comprises at least about 2,000 CD103+ cells per 10 million peripheral blood mononuclear cells. In some embodiments, the lymphocytes comprise at least about 20,000 CD103+ cells, wherein the CD103+ cells comprising the purity that is greater than or equal to about 80%. In some embodiments, the lymphocytes comprise at least about 2,000 CD103+ cells, wherein the CD103+ cells comprising the purity that is greater than or equal to about 80%. In some embodiments, the sample is enriched with the lymphocytes by greater than or equal to about 70%. In some embodiments, the sample is enriched with the lymphocytes by greater than or equal to about 80%. In some embodiments, the sample has a purity of the lymphocytes that is greater than or equal to about 70%. In some embodiments, the sample has a purity of the lymphocytes that is greater than or equal to about 80%.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIGS. 1A-1F show the working principle and the results of an experiment in which tumor-reactive lymphocytes (TRLs) in blood circulation are identified and isolated. FIG. 1A shows the working principle of tumor reactivity-mediated microfluidic cell sorting for analysis. Cells were firstly magnetically labeled based on their TCR reactivity with tumor antigen-derived MHC multimers. Then, magnetically labeled cells were separated from their counterparts by microfluidic cell sorting for downstream analysis. FIG. 1B shows the workflow of the identification via defined epitope models. CD8+ T cells in PBMC were classified as tumor-reactive and nonreactive populations based on their reactivity with multimers. Classified cells were compared to intratumoral CD8+ tumor-infiltrating lymphocytes (TILs) for clonal analysis. FIG. 1C shows the quantitation of tumor-reactive T cells in tumor and blood. FIG. 1D shows the quantitation of the sorting performance based on antibody and multimer through microfluidic sorting. FIG. 1E shows a comparison of clonal similarity among TIL, circulating TRL (CTRL), and peripheral blood monocyte (PBMC) by V-J usage profile. FIG. 1F shows analyses of the coverage of top 50 clones between each population. Unpaired t-test, mean±s.d., each dot represents a biological replicate.

FIGS. 2A-2I show the results of experiments used to characterize the molecular and phenotypic signature of cTRL during and post migration. FIG. 2A shows volcano plots showing the differential expression (DE) of genes when comparing the Fragments Per Kilobase of transcript per Million mapped reads (FPKM0 from PBMC and CTRL identified by multimer-based sorting from the B16 model. Key genes for CD8+ T cells are presented as a heatmap alongside. FIG. 2B shows a Gene Set Enrichment Analysis (GSEA) analysis of enriched immunological pathways. FIG. 2C shows an overlay of the shared, highly expressed genes from cTRLs in B16 and CT26 models. FIG. 2D shows an identification of tumor-reactivity in circulating CD8+CD103 populations. FIG. 2E shows the quantitation of tumor-reactive fraction in circulating CD8+CD103 populations. FIG. 2F shows a representative flow cytometric profile from the in vivo CTRL migration model. cTRLs migrated from donor tumor express CD45.2 while the endogenous TILs from the host express CD45.1. FIG. 2G shows the quantitation of the percentage of migrated cTRLs in the in vivo TIL migration model. FIG. 2H shows the CyTOF analysis of CD45.2+ CTRLs and CD45.1+ TILs for the expression of CD103 in AE17 models. FIG. 2I shows the quantitation of CyTOF data for the expression of CD103, CD69 and Programmed cell death protein 1 (PD-1) in CD45.2+ CTRLs and CD45.1+ TILs. Unpaired t-test, mean±s.d., each dot represents a biological replicate.

FIGS. 3A-3F show the results of murine model experiments in which CTRLs were shown to exhibit significant levels of activity against primary and metastasized tumors. FIG. 3A shows the experimental workflow. CTRL, CD8+CD103− PBMC and TIL were expanded 1-2 weeks in vitro before adoptive cell transfer. IL-2 was given daily for the first 3 days post cell transfer to boost lymphocyte proliferation. FIG. 3B shows the quantitation of tumor size, and survival rate. FIG. 3C shows the percentage of infiltrated CD8+ cells in s.c. B16 models treated by different T cells (n=5). FIG. 3D shows the representative bioluminescence images treated by different T cells (n=6) in induced 4T1 metastasis models. FIGS. 3E-3F show the quantitation of the total flux (FIG. 3E) and survival rate (FIG. 3F) in induced 4T1 metastasis models. *p<0.05, **p<0.01, unpaired t-test, mean±s.d., each dot represents a biological replicate.

FIGS. 4A-4G show the results of murine model experiments which demonstrate the synergistic effects of cTRLs and ICB/costimulatory molecules. FIG. 4A shows the quantitation of tumor size, survival rate, and percentage of infiltrated CD8+ cells in s.c. MC38 models treated by different therapeutic modalities (n=5). FIG. 4B is a gene expression analysis showing enriched pathways from upregulated RNAs, which reveal that αPD-1 and cTRLs generate different impacts to the immune responses within the tumor microenvironment in s.c. MC38 models. FIG. 4C shows the quantitation of CD4+ T cells and CD208+ dendritic cells post different therapeutic modalities. FIG. 4D shows rapid tumor rejection and formation of long-lasting TRLs were observed in cTRL-cured mice. FIG. 4E shows the quantitation of tumor size, survival rate, and percentage of infiltrated CD8+ cells in s.c. AE17 models treated by different therapeutic modalities (n=5). FIG. 4F shows the quantitation of lymphocyte subpopulations at the endpoint of treatment in endogenous (CD45.1+) populations in s.c. AE17 models. FIG. 4G shows the quantitation of CD103 expression in transferred cTRLs (CD45.2+) and endogenous lymphocytes (CD45.1+) in s.c. AE17 models. *p<0.05, **p<0.01, unpaired t-test, mean±s.d., each dot represents a biological replicate.

FIGS. 5A-5G show the results of experiments investigating the presence of CD103 CTRLs in human specimens. FIG. 5A shows the workflow of the co-culture assay to study the relationship between tumor-reactivity and CD103 on human PBMCs and examine the level of tumor specificity of isolated cTRLs. FIG. 5B shows the representative flowcytometric profile of IFN-γ secreting populations according to CD103 expression. This specific set of images is from PE71. FIG. 5C shows the quantitation of IFN-γ secreting populations in CD8+CD103 and CD8+CD103− cells across the patient cohort of malignant pleural effusion (MPE). FIG. 5D shows a representative flow cytometric profile of IFN-γ secreting populations in co-cultured populations. This specific set of images is from PE86. FIG. 5E shows the quantitation of IFN-γ secreting populations across a set of 18 patient samples. Tumor cells used in a coculture model to induce IFN-γ secretion were harvested either from tumor tissue (red) or malignant pleural effusions (blue). Fold enrichment was calculated by comparing the percentage of IFNγ+ cells in bulk CD8+ and CD8+CD103 populations post co-culture. FIG. 5F shows a comparison of clonal similarity among TILs, cTRLs and PBMC by V-J usage profile. CTRLs contains four TIL-derived major clones. FIG. 5G shows an analysis of the coverage of top 50 clones between each population.

FIGS. 6A-6B show the results of experiments in which human CTRLs were isolated and validated. FIG. 6A shows a representative flow cytometric profile of IFN-γ secreting populations in Solute Carrier Family 6 Member 19 (SLC6A19)+, CD103, and Systemic RNA Interference Deficient-1 Transmembrane Family Member 1 (SIDT1)+ populations of CD8+ T cells. This specific set of images is from PE96 and SMARTER (Mesothelioma trail) P29. FIG. 6B shows the quantitation of interferon-gamma (IFN-γ) secreting populations in SLC6A19+, CD103, and SIDT1+ populations of CD8+ T cells according to gate shown in FIG. 6A.

FIGS. 7A and 7B show the immunogenic epitopes that stimulate endogenous immune responses against the implanted tumors. FIG. 7A illustrates a schematic representation of the workflow for identifying Tumor-Resident Lymphocytes (TRLs) and circulating Tumor-Resident Lymphocytes (CTRLs) using defined epitope models. FIG. 7B shows the quantification of tumor-reactive T cells in both the tumor and blood samples.

FIGS. 8A-8C show supporting data for the immunogenic epitopes-mediated models. FIG. 8A shows the representative gating strategy used to identify infiltrated T cell in dissociated tumors. FIG. 8B shows the representative cytometric profile and quantitation of T cell infiltration and its impact on tumor weight at the end point for the B16OVA model. FIG. 8C shows the representative cytometric profile and quantitation of T cell infiltration and its impact on tumor weight at the end point for the CT26HA model.

FIGS. 9A-9C show the epitope-reactive T cells presented in blood circulation. FIG. 9A shows the representative gating strategy used to identify circulating T cells in RBC lysed blood. FIG. 9B shows the representative cytometric profile for the B16OVA model. FIG. 9C shows the representative cytometric profile for the CT26HA model.

FIGS. 10A-10F illustrate establishment of the in vivo TIL migration model. FIG. 10A illustrates the tumor transplantation model, where the donor mice carry CD45.2 isoform and the host mice carry CD45.1 isoform. FIG. 10B shows a photograph of a CD45.1+C57BL6 mouse carrying two tumors. Circles highlight the location of two tumors. FIG. 10C shows the representative flow cytometric profile from the in vivo TIL migration model (B16). TILs migrated from donor tumor express CD45.2 while the TILs from the host express CD45.1. FIG. 10D shows the representative flow cytometric profile from the in vivo CTRL migration model (AE17). CTRLs migrated from donor tumor express CD45.2 while the endogenous TILs from the host express CD45.1. FIG. 10E shows the quantitation of the percentage of migrated cTRLs in the in vivo TIL migration model. FIG. 10F shows the quantitation of CD45.2/CD45.1 percentage of migrated TILs in the in vivo TIL migration model.

FIGS. 11A-11E show microfluidic magnetic cell sorting. FIG. 11A illustrates immunomagnetic labeling workflow. FIG. 11B illustrates the chip design that favors the quantitative capture of cells based on its marker expression. ‘X’-shaped structures were introduced to improve the capture performance through forming low-velocity capture pockets in the microfluidic device. FIG. 11C shows the numerical simulation of the flow velocity profile within the microfluidic device. Capture pockets were formed near the edge of ‘X’-shaped structures, as indicated by the black/dark-red color. FIG. 11D shows the experimental validation of the cell capture profile within the microfluidic device. Almost all cells were captured on the capture pocket, proving the microfluidic device works as designed. FIG. 11E shows photographs of fabricated microfluidic devices along/on magnets. Food dye was used to visualize the channels.

FIG. 12 shows the representative workflow of microfluidic magnetic cell sorting according to some embodiments herein.

FIG. 13 shows the comparison of the performance of rare cell isolation based on multimer-labeling among FACS, MACS and microfluidics.

FIG. 14 represents enrichment plots of the GSEA data.

FIG. 15 shows the molecular signature of cTRLs from the CT26 model. Volcano plots show the differential expression (DE) of genes when comparing the FPKM from PBMC and CTRLs from the CT26 model. Key genes for CD8+ T cells were presented as a heatmap alongside.

FIG. 16 illustrates representative gating strategy used to identify cTRLs and endogenous TILs in the in vivo migration model.

FIGS. 17A-17D show a rapid expansion of cTRLs derived from MC-38 models using a feeder-based protocol. For the sake of visualization, CD8+ T cells from CD45.1+ mice were used a feeder to expand CD45.2+ CTRLs isolated from blood. FIG. 17A shows the representative flow cytometric profile of the CD45.2/CD45.1 ratio during the process of expansion. FIG. 17B shows the representative flow cytometric profile of the control samples from the CD8+ splenocytes isolated from CD45.1 or CD45.2 mice. FIG. 17C shows the quantitation of the fold of expansion. FIG. 17D shows a representative gating used for the data presented from FIGS. 17A-17C.

FIGS. 18A-18B show the rapid expansion of cTRLs derived from KPCY 6419c5 models using a feeder-based protocol. FIG. 18A illustrates the representative flow cytometric profile of the CD45.2/CD45.1 ratio during the process of expansion. FIG. 18B shows a quantitation of the fold of expansion.

FIGS. 19A-19C illustrate phenotyping of expanded cTRLs from MC-38 models. FIG. 19A represents the quantitation of relative mRNA expression of cTRLs compared to TILs via TaqMan probes. FIG. 19B represents the quantitation of protein expression of cTRLs and TILs for essential markers. FIG. 19C shows the representative flow cytometric profile of the data used in FIG. 19B.

FIG. 20 shows in vitro evaluation of the anti-tumor efficacy of cTRLs and TILs via co-culture killing assay.

FIGS. 21A-21E illustrate representative pictures and immunohistochemistry images of various tumor models post treatment. FIG. 21A shows B16F10 in WT C57BL6 mice on day 18. FIG. 21B shows LLC-1 in WT C57BL6 mice on day 15. FIG. 21C shows MC-38 in RAG−/−C57BL6 mice on day 18 and 32. FIG. 21D shows MC38 in CD45.1 C57BL6 mice on day 25. FIG. 21E shows AE17 in CD45.1 C57BL6 mice on day 15.

FIGS. 22A-22B represent an automatic quantitation of infiltrated CD8+ T cells from IHC slides using machine learning and image processing. FIG. 22A shows a random forest-based tumor classifier was trained by user-defined tumor/stroma/glass regions. The trained classifier was then applied to perform whole-slide segmentation to identify tumors. Stroma and glass regions were excluded in downstream analysis. It further illustrates representative decomposed images from the tumors. Number of CD8+ TILs were quantified by an automated cell counting algorithm using decomposed images. TILs were defined as hematoxylin+/warp red+. FIG. 22B shows quantification of tumor size, survival rate and percentage of infiltrated CD8+ cells in s.c. LLC-1 models in WT C57BL6 mice treated by different populations of lymphocytes (n=5).

FIG. 23 illustrates lung metastases in i.v. 4T1 models in nude mice at the endpoint treated by different T cells. (n=6, 6 layers for each animal L: Lung, T: Tumor), and quantitation of tumor size, survival rate and percentage of infiltrated CD8+ cells in s.c. MC38 models in RAG−/− C57BL6 mice treated by different therapeutic modalities (n=5).

FIGS. 24A-24B show CyTOF analyses of the immune landscape of infiltrated lymphocytes at the endpoint in s.c. AE17 models in CD45.1 C57BL6 mice treated by different therapeutic modalities. FIG. 24A shows the representative flow cytometric profile of the percentage of infiltrated CD3, CD4 and CD8 cells. FIG. 24B shows the representative flow cytometric profile of the percentage of CD8+PD-1+ cells and CD4+CD25+ cells.

FIG. 25 shows analysis of CD103 expression and its correlation with CD8+ T cell infiltration level using the TIMER algorithms. The level of CD8+ T cell infiltration is calculated using three prediction algorithm—CIBERSORT, MCPCOUNTER and QUANTISEQ. CD103 positively regulates the CD8+ T cell infiltration in many types of cancer types, including breast invasive carcinoma (BRCA), lung adenocarcinoma (LUAD) and colon adenocarcinoma (COAD).

FIGS. 26A-26B represent analyses of large-scale patient data using the TIDE algorithm. FIG. 26A shows a quantitation of adjusted death risk calculated by TIDE against different markers, such as CD103, ZNF683, TCF7 and SERPINB9. TCF7 is previously reported as an up-regulator for the improved therapeutic outcome, while SERPINB9 is reported as a down-regulator for therapeutic outcome. Data were visualized by a truncated violin plot and analyzed using an unpaired T-test. FIG. 26B shows analyses of the relationship between survival benefits and the lymphocytic ITGAE expression across multiple cancer types.

FIGS. 27A-27E show molecular and phenotypic signatures of cTRL. FIG. 27A shows key gene profiles for CD8+ T cells presented as a heatmap. FIGS. 27B-27C show CyTOF analysis of multimer-binding cTRLs (FIG. 27B) and expression levels of CD103, CD39, PD-1, and CD69 (FIG. 27C). FIGS. 27D-27E show the quantitation of the expression level of CD103, CD39, PD-1 and CD69 in cTRLs during migration (FIG. 27D) and gating schemes (FIG. 27E).

FIGS. 28A-28C illustrate CD103+ CTRL population signature in human PBMC. FIG. 28A schematically illustrates a workflow of the co-culture assay to study the relationship between tumor-reactivity and CD103 on human PBMCs. FIG. 28B shows the representative flow cytometric profile of IFN-γ secreting populations according to CD103 expression (from PE95 patient). FIG. 28C shows quantitation of IFN-γ secreting populations in CD8+CD103+ and CD8+CD103− cells across the patient cohort of malignant pleural effusion (MPE).

FIGS. 29A-29B illustrate the feeder-cell free expansion of T cells under different culture conditions, using initial seeding concentrations of 1,000 cells (FIG. 29A) or 10,000 cells (FIG. 29B).

### DETAILED DESCRIPTION

Disclosed herein, in some embodiments, are compositions, systems and kits comprising isolated and enriched populations of cells obtained from the sample of a subject, and methods of their isolation, enrichment, expansion, and use for treatment of a disease or a condition disclosed herein. In some embodiments, the sample comprises a fluid, such as peripheral blood. In some embodiments, the disease or the condition comprises cancer. In some embodiments, the subject is suspected of having a cancer. In some embodiments, the isolated and enriched populations of cells can target and eliminate cancer cells in a subject when administered to the subject as a therapy. In some embodiments, the isolated and enriched populations of cells are tumor-reactive lymphocytes (TRLs) or circulating TRLs (CTRLs) that can recognize a cancer antigen of the cancer and exhibit anti-cancer activity. In some cases, TRLs are present in the peripheral blood of a subject at a very low frequency, for example, as low as 0.002% of the peripheral blood T cell populations. The isolated and enriched TRLs (e.g., isolated and enriched CTRLs) described herein can be used as an alternative to tumor-infiltrating lymphocytes (TILs) in an adoptive cell therapy. The isolated and enriched TRLs disclosed herein can secrete interferon-gamma (IFN-γ). In some embodiments, the isolated and enriched TRLs can enter the peripheral blood from a primary tumor and accumulate in a secondary tumor. In some embodiments, a TRL can be characterized by having comparable reactivity to a TIL. In some embodiments, a TRL can share a clonotype with a TIL. In some embodiments, a population of TRLs can comprise 30%-85% of the top 50 clones presented in a population of intratumoral TILs. In some embodiments, TRLs described herein may have a tissue-resident-like (Tᵣₘ-like) phenotype. In some embodiments, the isolated and enriched population of TRLs can be enhanced to generate tumor-specific T cell therapies, such as chimeric antigen receptor (CAR)-T therapy or T-cell receptor (TCR) therapy.

In some embodiments, a TRL can express CD8, CD103, CD3, CD4, CD39, SLC6A19, SIDT1, or any combination thereof. In some embodiments, a TRL can express CD103. In some embodiments, a population of TRLs can comprise a CD103 signature. In some embodiments, a CD103 signature can define the population of TRLs. In some embodiments, a TRL can express SLC6A19, SIDT1, or a combination thereof. In some embodiments, a TRL can exhibit upregulated expression of TCF7, IL7R, LEF1, or a combination thereof. In some embodiments, a population of TRLs can comprise a SLC6A19+ and SIDT1+ signature. In some embodiments, a CD8+, CD103+, SLC6A19+, and SIDT1+ signature defines a population of TRLs. In some embodiments, the TRL can express CD3 or CD4. In some embodiments, the TRL can be a CD3+ pan T cell. In some embodiments the TRL can be a CD4+ helper T cell. In some embodiments, a TRL can express CD39. In some embodiments, the TRL can express CD39 and CD103. In some embodiments, a CD39+ and CD103+ signature defines a population of TRLs. In some embodiments, the TRL can express CD39, CD103, CD8, or a combination thereof. In some embodiments, a TRL can express CD8, CD103, SLC6A19, SIDT1 or a combination thereof. In some embodiments, a TRL can express CD3, CD103, SLC6A19, SIDT1 or a combination thereof. In some embodiments, a TRL can express CD4, CD103, SLC6A19, SIDT1 or a combination thereof. In some embodiments, a TRL can express CD4, CD39, CD103, SLC6A19, SIDT1 or a combination thereof.

Also disclosed herein are methods of isolating, enriching, and expanding a population of tumor reactive lymphocytes (TRLs) from a peripheral blood sample. Described herein are methods of isolating and enriching a population of TRLs that are present in a very low frequency (e.g., less than 0.002%) in the fluid (e.g., peripheral blood) using a microfluidic device. In some embodiments, isolating the population of TRLs can comprise magnetically separating a population of TRLs using a microfluidic device. In some embodiments, the microfluidic magnetic cell sorting can rely on the immunomagnetic labeling of the population of TRLs, followed by magnetic separation within the microfluidic device.

Further disclosed herein are methods of enhancing a population of the isolated and enriched TRLs comprising CD103, CD39, SLC6A19 and/or SIDT1 expressing lymphocytes by introducing a nucleotide that expresses a therapeutically enhancing polypeptide under conditions sufficient to produce the enhancing polypeptide by the cell. In some embodiments, the nucleotide can encode a T-cell receptor (TCR) or a chimeric antigen receptor (CAR). In some embodiments, the TCR or CAR is configured to recognize an antigen associated with a cancer.

Disclosed herein are methods of providing a cell therapy (e.g., an adoptive cell therapy) to a subject in need thereof, comprising: (a) obtaining a population of cells or therapeutically enhanced cells described herein; and (b) administering to the subject the population of cells, thereby providing the cell therapy. Also disclosed herein are compositions comprising a population of TRLs or therapeutically enhanced TRLs (e.g., to express a CAR). In some embodiments, the compositions can comprise a therapeutically effective amount of TRLs produced by any of the methods disclosed herein. In some embodiments, the compositions can be for use in treating a cancer.

Disclosed herein, in some embodiments, are compositions, systems, and kits for producing or utilizing therapeutically active cells described herein (e.g., enhanced TRLs). Compositions disclosed herein can comprise, in some embodiments, microfluidic devices for separating the population of population of TRLs from a fluid sample disclosed herein. In some embodiments, the compositions can comprise engineered T cell receptors (TCRs) and nucleic acid molecules encoding TCRs. In some embodiments, the compositions can comprise engineered chimeric antigen receptors (CARs) and nucleic acid molecules encoding CARs. In some embodiments, the fluid compositions disclosed herein are obtained from the subject in need of a treatment with the therapeutically active cells (e.g., autologous).

### I. Compositions

Disclosed herein are isolated and enriched populations of cells (e.g., T-cells) from the fluid (e.g., peripheral blood) of a subject (e.g., an individual with cancer) that can comprise tumor-reactive lymphocytes (TRLs) capable of targeting and eliminating cancer cells. Also disclosed herein are enhanced TRLs for use in therapeutic applications, such as synthetic chimeric antigen receptor (CAR) targeting tumor specific antigen. The compositions disclosed herein may be or comprise a polynucleotide encoding one or more components of the compositions disclosed herein, e.g., chimeric antigen receptor. The compositions disclosed herein may comprise in a pharmaceutical formulation, for example, in a formulation for administration to a subject disclosed herein. In some embodiments, a microfluidic device disclosed herein can be used to separate the population of population of TRLs from a fluid sample disclosed herein. In some embodiments, isolating and enriching tumor-reactive lymphocytes can involve a process that comprises magnetic separation of lymphocytes from the sample. This magnetic separation may involve flowing the lymphocytes through a microfluidic device channel that contains one or multiple magnetic capture zones.

### A. Tumor Reactive Lymphocytes

Disclosed herein are compositions comprising isolated and enriched tumor-reactive lymphocytes (TRLs) derived from a fluid sample (e.g., peripheral blood) that express Solute Carrier Family 6 Member 19 (SLC6A19) (NCBI Entrez Gene: 340024; UniProtKB/Swiss-Prot: Q695T7), Systemic RNA Interference Defective (SID) Protein 1 Transmembrane family member 1 (SIDT1) (NCBI Entrez Gene: 54847; UniProtKB/Swiss-Prot: Q9NXL6), Cluster of Differentiation 103 (CD103) (NCBI Entrez Gene: 3682; UniProtKB/Swiss-Prot: P38570), Cluster of Differentiation 39 (CD39) (NCBI Entrez Gene: 953; UniProtKB/Swiss-Prot: P49961) or any combination of thereof. In some embodiments, TRLs can be circulating tumor-reactive lymphocytes (CTRLs). In some embodiments, a population of TRLs (e.g., cTRLs) can comprise CD103 expressing cells. In some embodiments, a population of TRLs can comprise a CD103+ signature. In some embodiments, a population of TRLs can comprise CD39 expressing cells. In some embodiments, a population of TRLs can comprise a CD39+ signature. In some embodiments, a CD103+ signature can define the population of TRLs. In some embodiments, TRLs can express SLC6A19, SIDT1, or a combination thereof. In some embodiments, a population of TRLs can comprise a SLC6A19+ and/or SIDT1+ signature. In some embodiments, a SLC6A19+ and/or SIDT1+ signature can define the population of TRLs.

In some embodiments, the isolated and enriched TRLs may comprise about 100 cells to about 20,000 CD103 expressing cells per 10 million PBMCs (e.g., peripheral blood mononuclear cells). In some embodiments, the isolated and enriched TRLs may comprise about 100 cells to about 500 cells, from about 100 cells to about 1,000 cells, from about 100 cells to about 2,000 cells, from about 100 cells to about 3,000 cells, from about 100 cells to about 4,000 cells, from about 100 cells to about 5,000 cells, from about 100 cells to about 6,000 cells, from about 100 cells to about 8,000 cells, from about 100 cells to about 10,000 cells, from about 100 cells to about 15,000 cells, from about 100 cells to about 20,000 cells, from about 500 cells to about 1,000 cells, from about 500 cells to about 2,000 cells, from about 500 cells to about 3,000 cells, from about 500 cells to about 4,000 cells, from about 500 cells to about 5,000 cells, from about 500 cells to about 6,000 cells, from about 500 cells to about 8,000 cells, from about 500 cells to about 10,000 cells, from about 500 cells to about 15,000 cells, from about 500 cells to about 20,000 cells, from about 1,000 cells to about 2,000 cells, from about 1,000 cells to about 3,000 cells, from about 1,000 cells to about 4,000 cells, from about 1,000 cells to about 5,000 cells, from about 1,000 cells to about 6,000 cells, from about 1,000 cells to about 8,000 cells, from about 1,000 cells to about 10,000 cells, from about 1,000 cells to about 15,000 cells, from about 1,000 cells to about 20,000 cells, from about 2,000 cells to about 3,000 cells, from about 2,000 cells to about 4,000 cells, from about 2,000 cells to about 5,000 cells, from about 2,000 cells to about 6,000 cells, from about 2,000 cells to about 8,000 cells, from about 2,000 cells to about 10,000 cells, from about 2,000 cells to about 15,000 cells, from about 2,000 cells to about 20,000 cells, from about 3,000 cells to about 4,000 cells, from about 3,000 cells to about 5,000 cells, from about 3,000 cells to about 6,000 cells, from about 3,000 cells to about 8,000 cells, from about 3,000 cells to about 10,000 cells, from about 3,000 cells to about 15,000 cells, from about 3,000 cells to about 20,000 cells, from about 4,000 cells to about 5,000 cells, from about 4,000 cells to about 6,000 cells, from about 4,000 cells to about 8,000 cells, from about 4,000 cells to about 10,000 cells, from about 4,000 cells to about 15,000 cells, from about 4,000 cells to about 20,000 cells, from about 5,000 cells to about 6,000 cells, from about 5,000 cells to about 8,000 cells, from about 5,000 cells to about 10,000 cells, from about 5,000 cells to about 15,000 cells, from about 5,000 cells to about 20,000 cells, from about 6,000 cells to about 8,000 cells, from about 6,000 cells to about 10,000 cells, from about 6,000 cells to about 15,000 cells, from about 6,000 cells to about 20,000 cells, from about 8,000 cells to about 10,000 cells, from about 8,000 cells to about 15,000 cells, from about 8,000 cells to about 20,000 cells, from about 10,000 cells to about 15,000 cells, from about 10,000 cells to about 20,000 cells, or from about 15,000 cells to about 20,000 CD103 expressing cells per 10 million PBMCs.

In some embodiments, the isolated and enriched TRLs may comprise about 100 cells, about 500 cells, about 1,000 cells, about 2,000 cells, about 3,000 cells, about 4,000 cells, about 5,000 cells, about 6,000 cells, about 8,000 cells, about 10,000 cells, about 15,000 cells, or about 20,000 CD103 expressing cells per 10 million PBMCs. In some embodiments, the isolated and enriched TRLs may comprise at least about 100 cells, about 500 cells, about 1,000 cells, about 2,000 cells, about 3,000 cells, about 4,000 cells, about 5,000 cells, about 6,000 cells, about 8,000 cells, about 10,000 cells, or about 15,000 CD103 expressing cells per 10 million PBMCs. In some embodiments, the isolated and enriched TRLs may comprise at most about 500 cells, about 1,000 cells, about 2,000 cells, about 3,000 cells, about 4,000 cells, about 5,000 cells, about 6,000 cells, about 8,000 cells, about 10,000 cells, about 15,000 cells, or about 20,000 CD103 expressing cells per 10 million PBMCs.

In some embodiments, the isolated and enriched TRLs may comprise about 100 cells to about 20,000 SLA6A19 and/or SIDT1 expressing cells per 10 million PBMCs (e.g., peripheral blood mononuclear cells). In some embodiments, the isolated and enriched TRLs may comprise about 100 cells to about 500 cells, from about 100 cells to about 1,000 cells, from about 100 cells to about 2,000 cells, from about 100 cells to about 3,000 cells, from about 100 cells to about 4,000 cells, from about 100 cells to about 5,000 cells, from about 100 cells to about 6,000 cells, from about 100 cells to about 8,000 cells, from about 100 cells to about 10,000 cells, from about 100 cells to about 15,000 cells, from about 100 cells to about 20,000 cells, from about 500 cells to about 1,000 cells, from about 500 cells to about 2,000 cells, from about 500 cells to about 3,000 cells, from about 500 cells to about 4,000 cells, from about 500 cells to about 5,000 cells, from about 500 cells to about 6,000 cells, from about 500 cells to about 8,000 cells, from about 500 cells to about 10,000 cells, from about 500 cells to about 15,000 cells, from about 500 cells to about 20,000 cells, from about 1,000 cells to about 2,000 cells, from about 1,000 cells to about 3,000 cells, from about 1,000 cells to about 4,000 cells, from about 1,000 cells to about 5,000 cells, from about 1,000 cells to about 6,000 cells, from about 1,000 cells to about 8,000 cells, from about 1,000 cells to about 10,000 cells, from about 1,000 cells to about 15,000 cells, from about 1,000 cells to about 20,000 cells, from about 2,000 cells to about 3,000 cells, from about 2,000 cells to about 4,000 cells, from about 2,000 cells to about 5,000 cells, from about 2,000 cells to about 6,000 cells, from about 2,000 cells to about 8,000 cells, from about 2,000 cells to about 10,000 cells, from about 2,000 cells to about 15,000 cells, from about 2,000 cells to about 20,000 cells, from about 3,000 cells to about 4,000 cells, from about 3,000 cells to about 5,000 cells, from about 3,000 cells to about 6,000 cells, from about 3,000 cells to about 8,000 cells, from about 3,000 cells to about 10,000 cells, from about 3,000 cells to about 15,000 cells, from about 3,000 cells to about 20,000 cells, from about 4,000 cells to about 5,000 cells, from about 4,000 cells to about 6,000 cells, from about 4,000 cells to about 8,000 cells, from about 4,000 cells to about 10,000 cells, from about 4,000 cells to about 15,000 cells, from about 4,000 cells to about 20,000 cells, from about 5,000 cells to about 6,000 cells, from about 5,000 cells to about 8,000 cells, from about 5,000 cells to about 10,000 cells, from about 5,000 cells to about 15,000 cells, from about 5,000 cells to about 20,000 cells, from about 6,000 cells to about 8,000 cells, from about 6,000 cells to about 10,000 cells, from about 6,000 cells to about 15,000 cells, from about 6,000 cells to about 20,000 cells, from about 8,000 cells to about 10,000 cells, from about 8,000 cells to about 15,000 cells, from about 8,000 cells to about 20,000 cells, from about 10,000 cells to about 15,000 cells, from about 10,000 cells to about 20,000 cells, or from about 15,000 cells to about 20,000 SLA6A19 and/or SIDT1 expressing cells per 10 million PBMCs.

In some embodiments, the isolated and enriched TRLs may comprise about 100 cells, about 500 cells, about 1,000 cells, about 2,000 cells, about 3,000 cells, about 4,000 cells, about 5,000 cells, about 6,000 cells, about 8,000 cells, about 10,000 cells, about 15,000 cells, or about 20,000 SLA6A19 and/or SIDT1 expressing cells per 10 million PBMCs (e.g., peripheral blood mononuclear cells). In some embodiments, the isolated and enriched TRLs may comprise at least about 100 cells, about 500 cells, about 1,000 cells, about 2,000 cells, about 3,000 cells, about 4,000 cells, about 5,000 cells, about 6,000 cells, about 8,000 cells, about 10,000 cells, or about 15,000 cells per 10 million PBMCs. In some embodiments, the isolated and enriched TRLs may comprise at most about 500 cells, about 1,000 cells, about 2,000 cells, about 3,000 cells, about 4,000 cells, about 5,000 cells, about 6,000 cells, about 8,000 cells, about 10,000 cells, about 15,000 cells, or about 20,000 SLA6A19 and/or SIDT1 expressing cells per 10 million PBMCs.

In some embodiments, the purity of isolated and enriched TRLs (e.g., CD39, CD103, SLC6A19 and/or SIDT1 expressing cells) described herein can be about 10% to about 95%. In some embodiments, the purity of isolated and enriched TRLs (e.g., CD103, SLC6A19 and/or SIDT1 expressing cells) described herein can be about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 10% to about 95%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 15% to about 95%, about 20% to about 25%, about 20% to about 30%, about 20% to about 40%, about 20% to about 50%, about 20% to about 60%, about 20% to about 70%, about 20% to about 80%, about 20% to about 90%, about 20% to about 95%, about 25% to about 30%, about 25% to about 40%, about 25% to about 50%, about 25% to about 60%, about 25% to about 70%, about 25% to about 80%, about 25% to about 90%, about 25% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95%. In some embodiments, the purity of isolated and enriched TRLs (e.g., CD39, CD103, SLC6A19 and/or SIDT1 expressing cells) described herein can be about 10%, about 15%, about 20%, about 25%, about 30%, about 40%, about 50%, about 60%, about 70%, about 80%, about 90%, or about 95%. In some embodiments, the purity of isolated and enriched TRLs (e.g., CD39, CD103, SLC6A19 and/or SIDT1 expressing cells) described herein can be at least about 10%, about 15%, about 20%, about 25%, about 30%, about 40%, about 50%, about 60%, about 70%, about 80%, or about 90%. In some embodiments, the purity of isolated and enriched TRLs (e.g., CD39, CD103, SLC6A19 and/or SIDT1 expressing cells) described herein can be at most about 15%, about 20%, about 25%, about 30%, about 40%, about 50%, about 60%, about 70%, about 80%, about 90%, or about 95%.

In some embodiments, the purity of isolated and enriched TRLs (e.g., CD39, CD103, SLC6A19 or SIDT1 cells) described herein can be about 40%, about 50%, about 60%, about 70%, about 80%, about 85%, about 90%, about 95%, about 96%, about 97%, about 98%, or about 99%. In some embodiments, the purity of isolated and enriched TRLs described herein at least about 40%, about 50%, about 60%, about 70%, about 80%, about 85%, about 90%, about 95%, about 96%, about 97%, or about 98%. In some embodiments, the purity of isolated and enriched TRLs described herein can be at most about 50%, about 60%, about 70%, about 80%, about 85%, about 90%, about 95%, about 96%, about 97%, about 98%, or about 99%.

In some embodiments, the isolated and enriched TRLs described herein may comprise at least about 100 cells with a purity of at least about 90% of CD39, CD103, SLC6A19, and/or SIDT1 cells, about 500 cells with a purity of at least about 90% of CD39, CD103, SLC6A19, and/or SIDT1, about 1,000 cells with a purity of at least about 90% of CD39, CD103, SLC6A19, and/or SIDT1, about 2,000 cells with a purity of at least about 90% of CD39, CD103, SLC6A19, and/or SIDT1, about 3,000 cells with a purity of at least about 90% of CD39, CD103, SLC6A19, and/or SIDT1, about 4,000 cells with a purity of at least about 90% of CD39, CD103, SLC6A19, and/or SIDT1 cells, about 5,000 cells with a purity of at least about 90% of CD39, CD103, SLC6A19, and/or SIDT1 cells, about 6,000 cells with a purity of at least about 90% of CD39, CD103, SLC6A19, and/or SIDT1 cells, about 8,000 cells with a purity of at least about 90% of CD39, CD103, SLC6A19, and/or SIDT1 cells about 10,000 cells with a purity of at least about 90% of CD39, CD103, SLC6A19, and/or SIDT1 cells, about 15,000 cells with a purity of at least about 90% of CD39, CD103, SLC6A19, and/or SIDT1 cells, or about 20,000 cells with a purity of at least about 90% of CD39, CD103, SLC6A19, and/or SIDT1 cells per 10 million peripheral blood mononuclear cells (PBMCs).

In some embodiments, the isolated and enriched TRLs described herein may comprise at least about 100 cells with a purity of at least about 80% of CD39, CD103, SLC6A19, and/or SIDT1 cells, about 500 cells with a purity of at least about 80% of CD39, CD103, SLC6A19, and/or SIDT1, about 1,000 cells with a purity of at least about 80% of CD39, CD103, SLC6A19, and/or SIDT1, about 2,000 cells with a purity of at least about 80% of CD39, CD103, SLC6A19, and/or SIDT1, about 3,000 cells with a purity of at least about 80% of CD39, CD103, SLC6A19, and/or SIDT1, about 4,000 cells with a purity of at least about 80% of CD39, CD103, SLC6A19, and/or SIDT1 cells, about 5,000 cells with a purity of at least about 80% of CD39, CD103, SLC6A19, and/or SIDT1 cells, about 6,000 cells with a purity of at least about 80% of CD39, CD103, SLC6A19, and/or SIDT1 cells, about 8,000 cells with a purity of at least about 80% of CD39, CD103, SLC6A19, and/or SIDT1 cells about 10,000 cells with a purity of at least about 80% of CD39, CD103, SLC6A19, and/or SIDT1 cells, about 15,000 cells with a purity of at least about 80% of CD39, CD103, SLC6A19, and/or SIDT1 cells, or about 20,000 cells with a purity of at least about 80% of CD39, CD103, SLC6A19, and/or SIDT1 cells per 10 million PBMCs.

In some embodiments, the isolated and enriched TRLs described herein may comprise at least about 100 cells with a purity of at least about 70% of CD39, CD103, SLC6A19, and/or SIDT1 cells, about 500 cells with a purity of at least about 70% of CD39, CD103, SLC6A19, and/or SIDT1, about 1,000 cells with a purity of at least about 70% of CD39, CD103, SLC6A19, and/or SIDT1, about 2,000 cells with a purity of at least about 70% of CD39, CD103, SLC6A19, and/or SIDT1, about 3,000 cells with a purity of at least about 70% of CD39, CD103, SLC6A19, and/or SIDT1, about 4,000 cells with a purity of at least about 70% of CD39, CD103, SLC6A19, and/or SIDT1 cells, about 5,000 cells with a purity of at least about 70% of CD39, CD103, SLC6A19, and/or SIDT1 cells, about 6,000 cells with a purity of at least about 70% of CD39, CD103, SLC6A19, and/or SIDT1 cells, about 8,000 cells with a purity of at least about 70% of CD39, CD103, SLC6A19, and/or SIDT1 cells about 10,000 cells with a purity of at least about 70% of CD39, CD103, SLC6A19, and/or SIDT1 cells, about 15,000 cells with a purity of at least about 70% of CD39, CD103, SLC6A19, and/or SIDT1 cells, or about 20,000 cells with a purity of at least about 70% of CD39, CD103, SLC6A19, and/or SIDT1 cells per 10 million PBMCs.

In some embodiments, the isolated and enriched TRLs described herein may comprise at least about 100 cells with a purity of at least about 60% of CD39, CD103, SLC6A19, and/or SIDT1 cells, about 500 cells with a purity of at least about 60% of CD39, CD103, SLC6A19, and/or SIDT1, about 1,000 cells with a purity of at least about 60% of CD39, CD103, SLC6A19, and/or SIDT1, about 2,000 cells with a purity of at least about 60% of CD39, CD103, SLC6A19, and/or SIDT1, about 3,000 cells with a purity of at least about 60% of CD39, CD103, SLC6A19, and/or SIDT1, about 4,000 cells with a purity of at least about 60% of CD39, CD103, SLC6A19, and/or SIDT1 cells, about 5,000 cells with a purity of at least about 60% of CD39, CD103, SLC6A19, and/or SIDT1 cells, about 6,000 cells with a purity of at least about 60% of CD39, CD103, SLC6A19, and/or SIDT1 cells, about 8,000 cells with a purity of at least about 60% of CD39, CD103, SLC6A19, and/or SIDT1 cells about 10,000 cells with a purity of at least about 60% of CD39, CD103, SLC6A19, and/or SIDT1 cells, about 15,000 cells with a purity of at least about 60% of CD39, CD103, SLC6A19, and/or SIDT1 cells, or about 20,000 cells with a purity of at least about 60% of CD39, CD103, SLC6A19, and/or SIDT1 cells per 10 million PBMCs.

In some embodiments, the isolated and enriched TRLs described herein may comprise at least about 100 cells with a purity of at least about 50% of CD39, CD103, SLC6A19, and/or SIDT1 cells, about 500 cells with a purity of at least about 50% of CD39, CD103, SLC6A19, and/or SIDT1, about 1,000 cells with a purity of at least about 50% of CD39, CD103, SLC6A19, and/or SIDT1, about 2,000 cells with a purity of at least about 50% of CD39, CD103, SLC6A19, and/or SIDT1, about 3,000 cells with a purity of at least about 50% of CD39, CD103, SLC6A19, and/or SIDT1, about 4,000 cells with a purity of at least about 50% of CD39, CD103, SLC6A19, and/or SIDT1 cells, about 5,000 cells with a purity of at least about 50% of CD39, CD103, SLC6A19, and/or SIDT1 cells, about 6,000 cells with a purity of at least about 50% of CD39, CD103, SLC6A19, and/or SIDT1 cells, about 8,000 cells with a purity of at least about 50% of CD39, CD103, SLC6A19, and/or SIDT1 cells about 10,000 cells with a purity of at least about 50% of CD39, CD103, SLC6A19, and/or SIDT1 cells, about 15,000 cells with a purity of at least about 50% of CD39, CD103, SLC6A19, and/or SIDT1 cells, or about 20,000 cells with a purity of at least about 50% of CD39, CD103, SLC6A19, and/or SIDT1 cells per 10 million PBMCs.

In some embodiments, the isolated and enriched TRLs described herein may comprise at least about 100 cells with a purity of at least about 40% of CD39, CD103, SLC6A19, and/or SIDT1 cells, about 500 cells with a purity of at least about 40% of CD39, CD103, SLC6A19, and/or SIDT1, about 1,000 cells with a purity of at least about 40% of CD39, CD103, SLC6A19, and/or SIDT1, about 2,000 cells with a purity of at least about 40% of CD39, CD103, SLC6A19, and/or SIDT1, about 3,000 cells with a purity of at least about 40% of CD39, CD103, SLC6A19, and/or SIDT1, about 4,000 cells with a purity of at least about 40% of CD39, CD103, SLC6A19, and/or SIDT1 cells, about 5,000 cells with a purity of at least about 40% of CD39, CD103, SLC6A19, and/or SIDT1 cells, about 6,000 cells with a purity of at least about 40% of CD39, CD103, SLC6A19, and/or SIDT1 cells, about 8,000 cells with a purity of at least about 40% of CD39, CD103, SLC6A19, and/or SIDT1 cells about 10,000 cells with a purity of at least about 40% of CD39, CD103, SLC6A19, and/or SIDT1 cells, about 15,000 cells with a purity of at least about 40% of CD39, CD103, SLC6A19, and/or SIDT1 cells, or about 20,000 cells with a purity of at least about 40% of CD39, CD103, SLC6A19, and/or SIDT1 cells per 10 million PBMCs.

In some embodiments, the isolated and enriched TRLs described herein may comprise at least about 100 cells with a purity of at least about 30% of CD39, CD103, SLC6A19, and/or SIDT1 cells, about 500 cells with a purity of at least about 30% of CD39, CD103, SLC6A19, and/or SIDT1, about 1,000 cells with a purity of at least about 30% of CD39, CD103, SLC6A19, and/or SIDT1, about 2,000 cells with a purity of at least about 30% of CD39, CD103, SLC6A19, and/or SIDT1, about 3,000 cells with a purity of at least about 30% of CD39, CD103, SLC6A19, and/or SIDT1, about 4,000 cells with a purity of at least about 30% of CD39, CD103, SLC6A19, and/or SIDT1 cells, about 5,000 cells with a purity of at least about 30% of CD39, CD103, SLC6A19, and/or SIDT1 cells, about 6,000 cells with a purity of at least about 30% of CD39, CD103, SLC6A19, and/or SIDT1 cells, about 8,000 cells with a purity of at least about 30% of CD39, CD103, SLC6A19, and/or SIDT1 cells about 10,000 cells with a purity of at least about 30% of CD39, CD103, SLC6A19, and/or SIDT1 cells, about 15,000 cells with a purity of at least about 30% of CD39, CD103, SLC6A19, and/or SIDT1 cells, or about 20,000 cells with a purity of at least about 30% of CD39, CD103, SLC6A19, and/or SIDT1 cells per 10 million PBMCs.

In some embodiments, the population of TRLs can further express CD3, CD4, CD8, CD39, or any combination thereof. For example, the isolated and enriched TRLs can be CD3 and CD103 expressing cells. In some embodiments, the isolated and enriched TRLs can be CD4 and CD103 expressing cells. In some embodiments, the isolated and enriched TRLs can be CD8 and CD103 expressing cells. In some embodiments, the isolated and enriched TRLs can be CD39 and CD103 expressing cells. In some embodiments, the isolated and enriched TRLs can be CD3, CD39, and CD103 expressing cells. In some embodiments, the isolated and enriched TRLs can be CD4, CD39, and CD103 expressing cells. In some embodiments, the isolated and enriched TRLs can be CD8, CD39, and CD103 expressing cells. In some embodiments, the isolated and enriched TRLs can be CD3, SLC6A19, and SIDT1 expressing cells. In some embodiments, the isolated and enriched TRLs can be CD4, SLC6A19, and SIDT1 expressing cells. In some embodiments, the isolated and enriched TRLs can be CD8, SLC6A19, and SIDT1 expressing cells. In some embodiments, the isolated and enriched TRLs can be CD3, CD103, and SLC6A19 expressing cells. In some embodiments, the isolated and enriched TRLs can be CD4, CD103, and SLC6A19 expressing cells. In some embodiments, the isolated and enriched TRLs can be CD8, CD103, and SLC6A19 expressing cells. In some embodiments, the isolated and enriched TRLs can be CD3, CD103, SLC6A19, and SIDT1 expressing cells. In some embodiments, the isolated and enriched TRLs can be CD4, CD103, SLC6A19, and SIDT1 expressing cells. In some embodiments, the isolated and enriched TRLs can be CD8, CD103, SLC6A19, and SIDT1 expressing cells.

In some embodiments, a population of tumor-reactive lymphocytes (TRLs) may be found in a fluid sample of a patient having cancer. In some embodiments, a fluid sample may comprise a peripheral blood sample. In some embodiments, a fluid sample may be other biofluids, such as pleural effusion, ascites, and the like. In some embodiments, a fluid sample also be cord blood, bone marrow, lymph nodes, liver pleural effusion, thorax, abdominal cavity, synvial fluid, peritoneum, retroperitoneal space, thymus, and tumor.

### B. Enhanced TRLs

Disclosed herein, in some embodiments, are compositions comprising a population of isolated and enriched TRLs (e.g., isolated and enriched cTRLs) that are therapeutically enhanced (e.g., to express a CAR or a TCR). In some embodiments, genetic materials encoding either a cloned TCR or a synthetic chimeric antigen receptor (CAR) targeting tumor specific antigen can be introduced to the isolated and enriched TRLs described herein.

Generally, CARs are engineered fusion proteins constructed from antigen recognition, signaling, and costimulatory domains that may be expressed in T cells to reprogram the T cells to specifically target tumor cells. In some embodiments, a CAR is a recombinant polypeptide construct comprising at least an extracellular antigen binding domain, a transmembrane domain, and a cytoplasmic signaling domain comprising a functional signaling domain derived from a stimulatory molecule. Exogenous T cell receptors are similar to CARs in that they may be engineered to recognize an antigen (e.g., tumor antigen). In some embodiments the TCR is a recombinant polypeptide.

In some embodiments, the extracellular antigen binding domain is an antigen binding fragment of an antibody, or a functional portion thereof (e.g., an scFv) or a functional variant thereof. The specificity of the antigen binding domain may be modified to treat a variety of different disorders and may be mono-valent or multi-valent (e.g., di-valent, tri-valent). In some embodiments, the antigen binding domain comprises an scFv, and multivalent binding is provided by tandem addition of multiple scFvs bearing different antigen specificities. In some embodiments, the specificity and intended indication of the antigen binding matches that of any of the CAR-T constructs in contemporary clinical trials. For example, the specificity may include anti-CD19 (e.g., axicabtageneciloleucel for R/R diffuse large cell lymphoma; or Tisagenlecleucel, for R/R B cell ALL and non-Hodgkin lymphoma), anti-CD22 (e.g., for R/RB-ALL), anti-CD19/CD22 dual targeted (e.g., for R/R ALL), anti-CAIX (carbonic anhydrase 9), anti-PSMA (a.k.a FOLH1, e.g., for renal cell carcinoma), anti-MUC1 (e.g., for seminal vesicle carcinoma), anti-CD33 (e.g., for acute myeloid leukemia), anti-mesothelin mRNA (e.g., for adenocarcinoma and pleural mesothelioma), anti-FOLR1 (e.g., for metastatic ovarian cancer), anti-carcinoembryonic antigen (a.k.a. CEA, e.g., for CEA-expressing adenocarcinoma liver metastases), anti-IL13RA2 (e.g., for glioblastoma), anti-HER2 (e.g., for sarcoma), or any combination thereof. In some embodiments, one or more of the following antigens may be bound by the CAR-T construct: 1-40-β-amyloid, 4-1BB, 5AC, 5T4, 707-AP, A kinase anchor protein 4 (AKAP-4), activin receptor type-2B (ACVR2B), activin receptor-like kinase 1 (ALK1), adenocarcinoma antigen, adipophilin, adrenoceptor β 3 (ADRB3), AGS-22M6, α folate receptor, α-fetoprotein (AFP), AIM-2, anaplastic lymphoma kinase (ALK), androgen receptor, angiopoietin 2, angiopoietin 3, angiopoietin-binding cell surface receptor 2 (Tie 2), anthrax toxin, AOC3 (VAP-1), B cell maturation antigen (BCMA), B7-H3 (CD276), Bacillus anthracis anthrax, B-cell activating factor (BAFF), B-lymphoma cell, bone marrow stromal cell antigen 2 (BST2), Brother of the Regulator of Imprinted Sites (BORIS), C242 antigen, C5, CA-125, cancer antigen 125 (CA-125 or MUC16), Cancer/testis antigen 1 (NY-ESO-1), Cancer/testis antigen 2 (LAGE-1a), carbonic anhydrase 9 (CA-IX), Carcinoembryonic antigen (CEA), cardiac myosin, CCCTC-Binding Factor (CTCF), CCL11 (eotaxin-1), CCR4, CCR5, CD11, CD123, CD125, CD140a, CD147 (basigin), CD15, CD152, CD154 (CD40L), CD171, CD179a, CD18, CD19, CD2, CD20, CD200, CD22, CD221, CD23 (IgE receptor), CD24, CD25 (a chain of IL-2receptor), CD27, CD274, CD28, CD3, CD3 ε, CD30, CD300 molecule-like family member f (CD300LF), CD319 (SLAMF7), CD33, CD37, CD38, CD4, CD40, CD40 ligand, CD41, CD44 v7, CD44 v8, CD44 v6, CD5, CD51, CD52, CD56, CD6, CD70, CD72, CD74, CD79A, CD79B, CD80, CD97, CEA-related antigen, CFD, ch4D5, chromosome X open reading frame 61 (CXORF61), claudin 18.2 (CLDN18.2), claudin 6 (CLDN6), Clostridium difficile, clumping factor A, CLCA2, colony stimulating factor 1 receptor (CSF1R), CSF2, CTLA-4, C-type lectin domain family 12 member A (CLEC12A), C-type lectin-like molecule-1 (CLL-1 or CLECL1), C-X-C chemokine receptor type 4, cyclin B1, cytochrome P4501B1 (CYP1B1), cyp-B, cytomegalovirus, cytomegalovirus glycoprotein B, dabigatran, DLL4, DPP4, DR5, E. coli shiga toxin type-1, E. coli shiga toxin type-2, ecto-ADP-ribosyltransferase 4 (ART4), EGF-like module-containing mucin-like hormone receptor-like 2 (EMR2), EGF-like-domain multiple 7 (EGFL7), elongation factor 2 mutated (ELF2M), endotoxin, Ephrin A2, Ephrin B2, ephrin type-A receptor 2, epidermal growth factor receptor (EGFR), epidermal growth factor receptor variant III (EGFRvIII), episialin, epithelial cell adhesion molecule (EpCAM), epithelial glycoprotein 2 (EGP-2), epithelial glycoprotein 40 (EGP-40), ERBB2, ERBB3, ERBB4, ERG (transmembrane protease, serine 2 (TMPRSS2) ETS fusion gene), Escherichia coli, ETS translocation-variant gene 6, located on chromosome 12p (ETV6-AML), F protein of respiratory syncytial virus, FAP, Fc fragment of IgA receptor (FCAR or CD89), Fc receptor-like 5 (FCRL5), fetal acetylcholine receptor, fibrin II β chain, fibroblast activation protein α (FAP), fibronectin extra domain-B, FGF-5, Fms-Like Tyrosine Kinase 3 (FLT3), folate binding protein (FBP), folate hydrolase, folate receptor 1, folate receptor α, folate receptor β, Fos-related antigen 1, Frizzled receptor, Fucosyl GM1, G250, G protein-coupled receptor 20 (GPR20), G protein-coupled receptor class C group 5, member D (GPRC5D), ganglioside G2 (GD2), GD3 ganglioside, glycoprotein 100 (gp100), glypican-3 (GPC3), GMCSF receptor α-chain, GPNMB, GnT-V, growth differentiation factor 8, GUCY2C, heat shock protein 70-2 mutated (mut hsp70-2), hemagglutinin, Hepatitis A virus cellular receptor 1 (HAVCR1), hepatitis B surface antigen, hepatitis B virus, HER1, HER2/neu, HER3, hexasaccharide portion of globoH glycoceramide (GloboH), HGF, HHGFR, high molecular weight-melanoma-associated antigen (HMW-MAA), histone complex, HIV-1, HLA-DR, HNGF, Hsp90, HST-2 (FGF6), human papilloma virus E6 (HPV E6), human papilloma virus E7 (HPV E7), human scatter factor receptor kinase, human Telomerase reverse transcriptase (hTERT), human TNF, ICAM-1 (CD54), iCE, IFN-α, IFN-β, IFN-γ, IgE, IgE Fc region, IGF-1, IGF-1 receptor, IGHE, IL-12, IL-13, IL-17, IL-17A, IL-17F, IL-1B, IL-20, IL-22, IL-23, IL-31, IL-31RA, IL-4, IL-5, IL-6, IL-6 receptor, IL-9, immunoglobulin lambda-like polypeptide 1 (IGLL1), influenza A hemagglutinin, insulin-like growth factor 1 receptor (IGF-I receptor), insulin-like growth factor 2 (ILGF2), integrin α4β7, integrin β2, integrin α2, integrin α4, integrin α5β1, integrin α7β7, integrin αIIbβ3, integrin αvβ3, interferon α/β receptor, interferon γ-induced protein, Interleukin 11 receptor α (IL-11Rα), Interleukin-13 receptor subunit α-2 (IL-13Ra2 or CD213A2), intestinal carboxyl esterase, kinase domain region (KDR), KIR2D, KIT (CD117), L1-cell adhesion molecule (L1-CAM), legumain, leukocyte immunoglobulin-like receptor subfamily A member 2 (LILRA2), leukocyte-associated immunoglobulin-like receptor 1 (LAIR1), Lewis-Y antigen, LFA-1 (CD11a), LINGO-1, lipoteichoic acid, LOXL2, L-selectin (CD62L), lymphocyte antigen 6 complex, locus K 9 (LY6K), lymphocyte antigen 75 (LY75), lymphocyte-specific protein tyrosine kinase (LCK), lymphotoxin-α (LT-α) or Tumor necrosis factor-β (TNF-β), macrophage migration inhibitory factor (MIF or MMIF), M-CSF, mammary gland differentiation antigen (NY-BR-1), MCP-1, melanoma cancer testis antigen-1 (MAD-CT-1), melanoma cancer testis antigen-2 (MAD-CT-2), melanoma inhibitor of apoptosis (ML-IAP), melanoma-associated antigen 1 (MAGE-A1), mesothelin, mucin 1, cell surface associated (MUC1), MUC-2, mucin CanAg, myelin-associated glycoprotein, myostatin, N-Acetyl glucosaminyl-transferase V (NA17), NCA-90 (granulocyte antigen), nerve growth factor (NGF), neural apoptosis-regulated proteinase 1, neural cell adhesion molecule (NCAM), neurite outgrowth inhibitor (e.g., NOGO-A, NOGO-B, NOGO-C), neuropilin-1 (NRP1), N-glycolylneuraminic acid, NKG2D, Notch receptor, o-acetyl-GD2 ganglioside (OAcGD2), olfactory receptor 51E2 (OR51E2), oncofetal antigen (h5T4), oncogene fusion protein consisting of breakpoint cluster region (BCR) and Abelson murine leukemia viral oncogene homolog 1 (Abl) (bcr-abl), Oryctolagus cuniculus, OX-40, oxLDL, p53 mutant, paired box protein Pax-3 (PAX3), paired box protein Pax-5 (PAX5), pannexin 3 (PANX3), phosphate-sodium co-transporter, phosphatidylserine, placenta-specific 1 (PLAC1), platelet-derived growth factor receptor α (PDGF-R α), platelet-derived growth factor receptor β (PDGFR-β), polysialic acid, proacrosin binding protein sp32 (OY-TES1), programmed cell death protein 1 (PD-1), proprotein convertase subtilisin/kexin type 9 (PCSK9), prostase, prostate carcinoma tumor antigen-1 (PCTA-1 or Galectin 8), melanoma antigen recognized by T cells 1 (MelanA or MARTI), P15, P53, PRAME, prostate stem cell antigen (PSCA), prostate-specific membrane antigen (PSMA), prostatic acid phosphatase (PAP), prostatic carcinoma cells, prostein, Protease Serine 21 (Testisin or PRSS21), Proteasome (Prosome, Macropain) Subunit, β Type, 9 (LMP2), Pseudomonas aeruginosa, rabies virus glycoprotein, RAGE, Ras Homolog Family Member C (RhoC), receptor activator of nuclear factor kappa-B ligand (RANKL), Receptor for Advanced Glycation Endproducts (RAGE-1), receptor tyrosine kinase-like orphan receptor 1 (ROR1), renal ubiquitous 1 (RU1), renal ubiquitous 2 (RU2), respiratory syncytial virus, Rh blood group D antigen, Rhesus factor, sarcoma translocation breakpoints, sclerostin (SOST), selectin P, sialyl Lewis adhesion molecule (sLe), sperm protein 17 (SPA17), sphingosine-1-phosphate, squamous cell carcinoma antigen recognized by T Cells 1, 2, and 3 (SART1, SART2, and SART3), stage-specific embryonic antigen-4 (SSEA-4), Staphylococcus aureus, STEAP1, surviving, syndecan 1 (SDC1)+A314, SOX10, survivin, surviving-2B, synovial sarcoma, X breakpoint 2 (SSX2), T-cell receptor, TCR Γ Alternate Reading Frame Protein (TARP), telomerase, TEM1, tenascin C, TGF-β (e.g., TGF-β 1, TGF-β 2, TGF-β 3), thyroid stimulating hormone receptor (TSHR), tissue factor pathway inhibitor (TFPI), Tn antigen ((Tn Ag) or (GalNAcα-Ser/Thr)), TNF receptor family member B cell maturation (BCMA), TNF-α, TRAIL-R1, TRAIL-R2, TRG, transglutaminase 5 (TGS5), tumor antigen CTAA16.88, tumor endothelial marker 1 (TEM1/CD248), tumor endothelial marker 7-related (TEM7R), tumor protein p53 (p53), tumor specific glycosylation of MUC1, tumor-associated calcium signal transducer 2, tumor-associated glycoprotein 72 (TAG72), tumor-associated glycoprotein 72 (TAG-72)+A327, TWEAK receptor, tyrosinase, tyrosinase-related protein 1 (TYRP1 or glycoprotein 75), tyrosinase-related protein 2 (TYRP2), uroplakin 2 (UPK2), vascular endothelial growth factor (e.g., VEGF-A, VEGF-B, VEGF-C, VEGF-D, PIGF), vascular endothelial growth factor receptor 1 (VEGFR1), vascular endothelial growth factor receptor 2 (VEGFR2), vimentin, v-myc avian myelocytomatosis viral oncogene neuroblastoma derived homolog (MYCN), von Willebrand factor (VWF), Wilms tumor protein (WT1), X Antigen Family, Member 1A (XAGE1), β-amyloid, and k-light chain.

In some embodiments, the transmembrane domain of the CAR is a domain that localizes the CAR to the correct membrane location and stabilizes its structure. Suitable transmembrane domains can include the transmembrane region(s) of alpha, beta or zeta chain of the T-cell receptor; or a transmembrane region from CD28, CD3 epsilon, CD3ζ, CD45, CD4, CD5, CD8alpha, CD9, CD16, CD22, CD33, CD37, CD64, CD80, CD86, CD134, CD137, CD154 or a functional portion or functional variant thereof. Alternatively, the transmembrane domain can be synthetic, and can comprise hydrophobic residues such as leucine and valine. In some embodiments, a triplet of phenylalanine, tryptophan and valine is found at one or both termini of a synthetic transmembrane domain. Optionally, a short oligonucleotide or polypeptide linker, in some embodiments, between 2 and 10 amino acids in length may form the linkage between the transmembrane domain and the cytoplasmic signaling domain of a CAR. In some embodiments, the linker is a glycine-serine linker. In some embodiments, the CAR comprises a cytoplasmic signaling domain comprising a functional signaling domain derived from a stimulatory molecule. In some embodiments, the stimulatory molecule is a stimulatory receptor molecule. In some embodiments, the stimulatory receptor molecule is a stimulatory receptor molecule of an adaptive immune cell. In some embodiments, the stimulatory molecule is the zeta chain associated with the T cell receptor complex. In some embodiments, the stimulatory molecule is e.g., FCERIG, Fc gamma RIIa, FcR beta (Fc Epsilon Rib), CD3 gamma, CD3 delta, CD3 epsilon, CD79a, CD79b, DAP10, or DAP1, or a functional portion or functional variant thereof. In some embodiments, the intracellular signaling domain comprises one or more functional signaling domains derived from at least one costimulatory molecule. In some embodiments, the costimulatory molecule comprises 4-1BB (i.e., CD137), CD27, CD28 CD30, CD40, PD-1, CD2, CD7, CD258, NKG2C, B7-H3, a ligand that binds to CD83, ICAM-1, LFA-1 (CD1 Ia/CD18), ICOS, a functional portion or functional variant thereof, or a combination thereof. In some embodiments, the CAR comprises a leader sequence at the amino-terminus (N-terminus) of the CAR fusion protein. In some embodiments, the CAR comprises a signal peptide sequence at the N-terminus of the extracellular antigen recognition domain, wherein the signal peptide sequence is optionally cleaved from the antigen recognition domain (e.g., a scFv), or a functional portion or functional variant thereof during cellular processing and localization of the CAR to the cellular membrane

In some embodiments, a CAR disclosed herein is a first-, second-, third-, or fourth-generation CAR system, a functional variant thereof, or a combination thereof. In some embodiments, a first-generation CAR comprises an antigen binding domain with specificity for a particular antigen (e.g., an antibody or antigen-binding fragment thereof, such as an scFv, a Fab fragment, a VHH domain, or a VH domain of a heavy-chain only antibody), a transmembrane domain derived from an adaptive immune receptor (e.g., the transmembrane domain from the CD28 receptor or a functional portion or functional variant thereof), and a signaling domain derived from an adaptive immune receptor (e.g., the three ITAM domains derived from the intracellular region of the CD3ζ receptor or FcεRIγ or a functional portion or functional variant thereof). In some embodiments, a second-generation CAR construct comprises the elements of the first-generation CAR and an addition of a co-stimulatory domain to the intracellular signaling domain portion of the CAR (e.g., derived from co-stimulatory receptors that act alongside T-cell receptors such as CD28, CD137/4-1BB, and CD134/OX40 or a functional portion or functional variant thereof). In some embodiments, the co-stimulatory domain abrogates the need for administration of IL-2 alongside a first-generation CAR. In some embodiments, a third-generation CAR comprises the elements of a first-generation CAR with the addition of multiple co-stimulatory domains to the intracellular signaling domain portion of the CAR (e.g., CD32-CD28-OX40, or CD32-CD28-41BB or a functional portion or functional variant thereof). In some embodiments, fourth-generation CAR comprises the elements of a second- or third-generation CARs with the addition of an activating cytokine (e.g., IL-12, IL-23, or IL-27 or a functional portion or functional variant thereof) to the intracellular signaling portion of the CAR (typically between one or more of the costimulatory domains and the CD3ζ ITAM domain or a functional portion or functional variant thereof) or under the control of a CAR-induced promoter (e.g., the NFAT/IL-2 minimal promoter or a functional portion or functional variant thereof).

The isolated and enriched TRLs may be manufactured to express a CAR by variety approaches known to those skilled in the art, which generally include the following steps: isolating a subject's T cells, activating said T cells, transducing said T cells with a CAR transgene, and expanding said transduced T Cells for the required number for cell therapy. In some embodiments, the T cells (e.g., isolated and enriched TRLs such as CD103, SLC6A19 and/or SIDT1 expressing cells) can be isolated using any of the methods disclosed herein. In some embodiments, transducing a cell with a CAR transgene comprises introducing the cell to a nucleotide that expresses a CAR under conditions sufficient to produce the CAR by the cell. Methods for introducing genetically engineered components, such as CARs, to T-Cells are well known to skilled artisans and may be used to produce the CARs disclosed herein. Exemplary methods for transferring a nucleic encoding the CAR, can include viral transduction, e.g., via retroviral transduction or lentiviral transduction, transposon, and electroporation-mediated methods.

Also provided are polynucleotides encoding the compositions disclosed herein. In some embodiments, the vector can comprise a backbone and the polynucleotides encoding a chimeric antigen receptor (CAR), wherein the CAR comprises (a) antigen binding domain; (b) a transmembrane domain; (c) a costimulatory signaling domain (e.g., 4-1BB or CD28, or both); and/or (d) a CD3 zeta signaling domain. In some embodiments, a vector can comprise one or more of the polynucleotides disclosed herein. In some embodiments, the vector can be a plurality of vectors. In some embodiments, the polynucleotides encoding the CAR can be transferred to a TRL (e.g., cTRL) using a lentiviral vector. In some embodiments, the nucleotide encoding the CAR can be transferred to a TRL using a retroviral vector. In some cases, the vector can be a non-viral vector. In some embodiments, the non-viral vector can be a Sleeping Beauty transposon. In some embodiments, the vector comprises a plasmid. In some cases, each vector described herein can comprise an expression plasmid.

In some embodiments, the polynucleotides encoding the CAR can be cloned into a vector comprising lentiviral backbone components. Exemplary backbone components can include, but are not limited to, pFUGW, and pSMPUW. The pFUGW lentiviral vector backbone is a self-inactivating (SIN) lentiviral vector backbone and has unnecessary HIV-1 viral sequences removed resulting in reduced potential for the development of neoplasia, harmful mutations, and regeneration of infectious particles. In some embodiments, the CAR can be under the control of an inducible promoter. In some cases, an inducible promoter can be a small molecule ligand-inducible two polypeptide ecdysone receptor-based gene switch. In some embodiments, the CAR can be under the control of a constitutive promoter.

Provided herein is a system for expressing a CAR in a TRL (e.g., cTRLs), wherein the system comprises one or more vectors encoding polynucleotides disclosed herein. In some cases, the system can further comprise a nucleic acid encoding at least one additional gene. In some embodiments, the additional gene can comprise a cytokine. In some embodiments, the cytokine can comprise at least one of IL-2, IL-15, IL-12, IL-21, and a fusion of IL-15 and IL-15Ra, or a functional portion or functional variant thereof. In some embodiments, the cytokine can be in secreted form. In some embodiments, the cytokine is in membrane bound form.

### C. Pharmaceutical Formulations

Described herein are pharmaceutical formulations comprising the TRLs (e.g., CTRLs) or the enhanced TRLs (e.g., enhanced cTRLs) described herein. In some embodiments, a pharmaceutical formulation can comprise TRLs or enhanced TRLs comprising a population of SLC6A19+ lymphocytes, SIDT1+ lymphocytes, CD103+ lymphocytes, CD39+ lymphocytes, or combination thereof. In some embodiments, the pharmaceutical formulations further comprise a pharmaceutically acceptable: carrier, excipient, diluent, or nebulized inhalant.

In some embodiments, the pharmaceutical formulations include two or more active agents, or two or more therapeutic agents as disclosed herein. In some embodiments, the two or more active agents are contained in a single dosage unit such as, for example, when the TRL or the enhanced TRLs (e.g., CAR or TCR) comprises or are administered with one or more therapeutic agents. In embodiments, the two or more active agents are contained in separate dosage units such as when the TRLs or the enhanced TRLs (e.g., CAR or TCR) are administered separately from an additional therapeutic agent or adjuvant. In some embodiments, the active agents that may be, in some embodiments, the additional therapeutic agent which includes a chemotherapeutic agent, cytotoxic agent, cytokine, growth-inhibitory agent, anti-hormonal agent, anti-angiogenic agent, cardio protectant, and/or checkpoint inhibitor. Non-limiting checkpoint inhibitor includes IMP321/Eftilagimod alpha (Immutep), Relatlimab BMS-986016, Ipilimumab (Yervoy), Pembrolizumab (Keytruda), Nivolumab (Opdivo), Cemiplimab (Libtayo), Atezolizumab (Tecentriq), Avelumab (Bavencio), Durvalumab (Imfinzi), Ipilimumab (Yervoy), LAG525, MK-4280, Irinotecan, Oxaliplatin, REGN3767, TSR-033, BI754111, Sym022, FS118 (a bi-specific anti-LAG3/PD-L1 antagonistic mAb), MGD013 (a bi-specific anti-LAG3/PD-1 antagonistic mAb), TSR-022, Niraparib, Bevacizumab, MBG453, Decitabine, Spartalizumab, Sym023, INCAGN2390, LY3321367, Ramucirumab, Abemaciclib, Merestinib, BMS-986258, SHR-1702, Camrelizumab, MK-7684, Etigilimab/OMP-313 M32, Tiragolumab/MTIG7192A/RG-6058, BMS-986207, AB-154, ASP-8374, JNJ-61610588, CA-170d, Enoblituzumab/MGA271, MGD009, I-8H9/omburtamab, Trastuzumab, MGD013 (Anti-PD-1, anti-LAG-3 dual checkpoint inhibitor), BGB-A1217, CM-24 (MK-6018), BMS 986178, MEDI6469, PF-04518600, GSK3174998, MOXR0916, Utomilimab (PF-05082566), Urelumab (BMS-663513) ES101, BMS-986156, TRX-518, AMG 228, JTX-2011, GSK3359609, BMS-986226, MEDI-570, or Varlilumab (CDX-1127).

The pharmaceutical formulations described herein may be formulated for administration to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended-release formulations, pulsatile release formulations, multiparticulate formulations, and mixed immediate and controlled release formulations.

The pharmaceutical formulations including a therapeutic agent may be manufactured in a conventional manner such as, by way of example only, by means of conventional mixing, dissolving, granulating, dragee-making, levigating, emulsifying, encapsulating, entrapping or compression processes.

The pharmaceutical formulations may include at least an exogenous therapeutic agent as an active ingredient in free-acid or free-base form, or in a pharmaceutically acceptable salt form. In addition, the methods and compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In certain embodiments, pharmaceutical formulations provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, pharmaceutical formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, I about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical formulations described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents such as parabens, chlorobutanol, phenol, sorbic acid, and the like. In some embodiments, it is desirable to include isotonic agents such as sugars, sodium chloride, and the like. Prolonged absorption of the injectable pharmaceutical form may be brought about by the use of agents delaying absorption such as aluminum monostearate and gelatin.

For intravenous injections or drips or infusions, a pharmaceutical formulations described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Pharmaceutical formulations for injection may be presented in unit dosage form, e.g., in ampoules or in multi dose containers, with an added preservative. The composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical formulations described herein are conveniently delivered in the form of an aerosol spray presentation from pressurized packs or nebulizers, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of such as, by way of example only, gelatin for use in an inhaler or insufflator may be formulated containing a powder mix of the therapeutic agent described herein and a suitable powder base such as lactose or starch. Formulations that include a composition are prepared as solutions in saline, employing benzyl alcohol or other suitable preservatives, fluorocarbons, and/or other solubilizing or dispersing agents known in the art. Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compositions described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, the pharmaceutical formulations of the exogenous therapeutic agents are in the form of a capsules, including push fit capsules made of gelatin, as well as soft, sealed capsules made of gelatin and a plasticizer such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole, or the capsule is opened, and the contents sprinkled on food prior to eating.

Pharmaceutical formulations for oral administration are in dosages suitable for such administration. In one aspect, solid oral dosage forms are prepared by mixing a composition with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the composition is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating may provide a delayed release of a therapeutic agent from the formulation. In other embodiments, the film coating aids in patient compliance. Film coatings typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

Buccal formulations are administered using a variety of formulations known in the art. In addition, the buccal dosage forms described herein may further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a pharmaceutical formulations is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a composition described herein is in a form suitable for parenteral injection as a sterile suspensions, solutions or emulsions in oily or aqueous vehicles, and contain formulatory agents such as suspending, stabilizing and/or dispersing agents. The compositions for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

In some embodiments, the compositions are provided that include particles of a therapeutic agent and at least one dispersing agent or suspending agent for oral administration to a subject. The formulations may be a powder and/or granules for suspension, and upon admixture with water, a substantially uniform suspension is obtained.

Furthermore, the pharmaceutical formulations optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the pharmaceutical formulations optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other the pharmaceutical formulations optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state for at least 4 hours. In one embodiment, an aqueous suspension is resuspended into a homogenous suspension by physical agitation lasting less than 1 minute. In still another embodiment, no agitation is necessary to maintain a homogeneous aqueous dispersion.

An aerosol formulation for nasal administration is generally an aqueous solution designed to be administered to the nasal passages in drops or sprays. Nasal solutions may be similar to nasal secretions in that they are generally isotonic and slightly buffered to maintain a pH of about 5.5 to about 6.5, although pH values outside of this range may additionally be used. Antimicrobial agents or preservatives may also be included in the formulation.

An aerosol formulation for inhalations and inhalants may be designed so that the agent or combination of agents is carried into the respiratory tree of the subject when administered by the nasal or oral respiratory route. Inhalation solutions may be administered, for example, by a nebulizer. Inhalations or insufflations, comprising finely powdered or liquid drugs, may be delivered to the respiratory system as a pharmaceutical aerosol of a solution or suspension of the agent or combination of agents in a propellant, e.g., to aid in disbursement. Propellants may be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants may include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Hydrocarbon propellants useful include, for example, propane, isobutane, n-butane, pentane, isopentane and neopentane. A blend of hydrocarbons may also be used as a propellant. Ether propellants include, for example, dimethyl ether as well as the ethers. An aerosol formulation may also comprise more than one propellant. For example, the aerosol formulation comprises more than one propellant from the same class such as two or more fluorocarbons; or more than one, more than two, more than three propellants from different classes such as a fluorohydrocarbon and a hydrocarbon. The compositions of the present disclosure may also be dispensed with a compressed gas, e.g., an inert gas such as carbon dioxide, nitrous oxide or nitrogen.

Aerosol formulations may also include other components, for example, ethanol, isopropanol, propylene glycol, as well as surfactants or other components such as oils and detergents. These components may serve to stabilize the formulation and/or lubricate valve components.

The aerosol formulation may be packaged under pressure and may be formulated as an aerosol using solutions, suspensions, emulsions, powders and semisolid preparations. For example, a solution aerosol formulation comprises a solution of an agent such as a transporter, carrier, or ion channel inhibitor in (substantially) pure propellant or as a mixture of propellant and solvent. The solvent may be used to dissolve the agent and/or retard the evaporation of the propellant. Solvents may include, for example, water, ethanol and glycols. Any combination of suitable solvents may be use, optionally combined with preservatives, antioxidants, and/or other aerosol components.

An aerosol formulation may be a dispersion or suspension. A suspension aerosol formulation comprises a suspension of an agent or combination of agents, e.g., a transporter, carrier, or ion channel inhibitor, and a dispersing agent. Dispersing agents may include, for example, sorbitan trioleate, oleyl alcohol, oleic acid, lecithin and corn oil. A suspension aerosol formulation may also include lubricants, preservatives, antioxidant, and/or other aerosol components.

An aerosol formulation may similarly be formulated as an emulsion. An emulsion aerosol formulation may include, for example, an alcohol such as ethanol, a surfactant, water and a propellant, as well as an agent or combination of agents, e.g., a transporter, carrier, or ion channel. The surfactant used may be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

### II. Methods

Disclosed herein are methods of isolating, enriching, expanding, and using the tumor reactive lymphocytes (TRLs) or the enhanced TRLs of the present disclosure. Also provided are methods of producing the enhanced TRLs from the TRLs of the present disclosure. Disclosed herein are methods of isolating and expanding a population of tumor reactive lymphocytes (TRLs) from a fluid sample (e.g., peripheral blood) of a subject. In some embodiments, the subject may have a cancer or may be suspected of having cancer. For example, the subject may have a cancer in a breast tissue, a renal tissue, a cervical tissue, a lung tissue, an ovarian tissue, or a skin tissue. The resulting TRLs may be used, for example, in an adoptive cell therapy. In some embodiments, methods disclosed herein comprise isolating TRLs from the peripheral blood of a subject, wherein the TRL population comprises CD103 expressing lymphocytes (CD103+ lymphocytes). In some embodiments, methods described herein can comprise isolating from the peripheral blood of a subject a population of TRLs expressing CD103, CD39, SLC6A19, and/or SIDT1. In some embodiments, isolating the population of TRLs comprise magnetically separating a population of TRLs. In some embodiments, the methods of isolating the population of TRLs comprises magnetically separating the population of TRLs using a microfluidic device. In some embodiments, the microfluidic magnetic cell sorting relies on the immunomagnetic labeling of the population of TRLs, followed by magnetic separation within the microfluidic device.

Also disclosed herein are methods of producing a population of enhanced TRLs. In some embodiments, methods of enhancing a population of TRLs comprise isolating and expanding population of CD103, CD39, SLC6A19 and/or SIDT1 expressing lymphocytes to a nucleotide that expresses a therapeutically enhancing polypeptide under conditions sufficient to produce the enhancing polypeptide by the cell. In some embodiments, the nucleotide encodes a chimeric antigen receptor (CAR). In some embodiments, the CAR is configured to recognize an antigen associated with a cancer.

Also disclosed herein are methods of providing a cell therapy (e.g., an adoptive cell therapy) comprising the TRLs or the enhanced TRLs of the present disclosed to a subject in need thereof. In some embodiments, the methods comprises (a) obtaining a population of cells or therapeutically enhanced cells described herein; and (b) administering to the subject the population of cells, thereby providing the cell therapy. In some embodiments, the isolated TRLs are expanded using any of the methods disclosed herein. In some embodiments, autologous cells are preferred over allogeneic cells because of because of their inherent heterogeneity that maximizes the tumor-recognizing T cell receptors (TCRs) while minimizing off-tissue effects. In some embodiments, the subject has a cancer.

### A. Methods of Isolating Tumor Reactive Lymphocytes

Disclosed herein, in some embodiments, is a method of isolating a population of TRLs (e.g., cTRLs) comprising obtaining a peripheral blood sample from a subject comprising lymphocytes; and separating from the sample a population of CD103, CD39, SLC6A19 and/or SIDT1 expressing TRLs. In some embodiments, separating from the sample a population of CD103, CD39, SLC6A19 and/or SIDT1 expressing TRLs may comprise magnetically separating from the sample. In some embodiments, magnetically separating from the sample a population of CD103, CD39, SLC6A19 and/or SIDT1 expressing lymphocytes may comprise immunomagnetically labeling the population of TRLs followed by magnetic separation within the microfluidic device.

In some embodiments, immunomagnetically labeling a population of TRLs can comprise attaching—directly or indirectly—a magnetic label to a surface maker of at least one of the TRLs. In some embodiments, the surface marker can be CD103, CD39, SLC6A19, SIDT1 or a combination thereof. In some embodiments, the surface marker can further comprise CD3, CD4, CD8 or a combination thereof. In some embodiments, the surface marker can be CD103. In some embodiments, the surface marker can be CD39. In some embodiments, the surface marker can be SLC6A19. In some embodiments, the surface marker can be SIDT1. In some embodiments, the surface marker can comprise CD3, CD4, CD8, CD39, CD103, SLC6A19, SIDT1, or a combination thereof. In some embodiments, the TRLs are labeled by an antibody targeting a surface marker of interest (e.g., an anti-CD103 antibody). In some embodiments, the cells are labeled by a multimer targeting a surface marker of interest (e.g., an MHC multimer). In some embodiments, the antibody or multimer is conjugated to a magnetic micro or nano particle (MNP). In some embodiments, the antibody or multimer is labeled by a secondary antibody conjugated with a MNP. In some embodiments, after labeling, the TRLs obtain a level of magnetization as a function of the expression level of the surface marker recognized by the antibody or multimer.

In some embodiments, the microfluidic device is configured to isolate the population of TRLs from a population of non-TRLs based on the levels of magnetization exhibited by the TRLs. For example, the microfluidic device may comprise a sorting chamber comprising several separated zones with varying heights. In some embodiments, in each zone, microstructures are patterned to generate capture pockets that create low-velocity zones for trapping magnetically labeled TRLs. In some embodiments, during operation, the microfluidic device is sandwiched by arrays of magnets that generate constant magnetic fields in the sorting chamber and is connected to a syringe pump for fluidic processing. Without being bound by any theory, when the cells are added to the device, they experience two major forces—the magnetic force generated by the interaction between MNPs and constant magnetic field, and a fluidic drag force which is defined by the fluidic velocity in a specific zone. When the magnetic force overcomes the drag force, a cell acquires enough trapping force to stay in a specific zone. Otherwise, a cell would be flush into the next zone with a lower drag force, and eventually into the syringe if it cannot be captured by any zone. After sorting, captured cells at each zone are recovered by removing the external magnets.

In some embodiments, the microfluidic device exhibits about 20% to about 98% capture efficiency when capturing a population of rare cells (e.g., a population of TRLs). In some embodiments, the microfluidic device exhibits about 20% to about 30%, about 20% to about 40%, about 20% to about 50%, about 20% to about 60%, about 20% to about 70%, about 20% to about 80%, about 20% to about 90%, about 20% to about 92%, about 20% to about 94%, about 20% to about 96%, about 20% to about 98%, about 30% to about 40%, about 30% to about 50%, about 30% to about 60%, about 30% to about 70%, about 30% to about 80%, about 30% to about 90%, about 30% to about 92%, about 30% to about 94%, about 30% to about 96%, about 30% to about 98%, about 40% to about 50%, about 40% to about 60%, about 40% to about 70%, about 40% to about 80%, about 40% to about 90%, about 40% to about 92%, about 40% to about 94%, about 40% to about 96%, about 40% to about 98%, about 50% to about 60%, about 50% to about 70%, about 50% to about 80%, about 50% to about 90%, about 50% to about 92%, about 50% to about 94%, about 50% to about 96%, about 50% to about 98%, about 60% to about 70%, about 60% to about 80%, about 60% to about 90%, about 60% to about 92%, about 60% to about 94%, about 60% to about 96%, about 60% to about 98%, about 70% to about 80%, about 70% to about 90%, about 70% to about 92%, about 70% to about 94%, about 70% to about 96%, about 70% to about 98%, about 80% to about 90%, about 80% to about 92%, about 80% to about 94%, about 80% to about 96%, about 80% to about 98%, about 90% to about 92%, about 90% to about 94%, about 90% to about 96%, about 90% to about 98%, about 92% to about 94%, about 92% to about 96%, about 92% to about 98%, about 94% to about 96%, about 94% to about 98%, or about 96% to about 98% capture efficiency when capturing a population of rare cells (e.g., a population of TRLs). In some embodiments, the microfluidic device exhibits about 20%, about 30%, about 40%, about 50%, about 60%, about 70%, about 80%, about 90%, about 92%, about 94%, about 96%, or about 98% capture efficiency when capturing a population of rare cells (e.g., a population of TRLs). In some embodiments, the microfluidic device exhibits at least about 20%, about 30%, about 40%, about 50%, about 60%, about 70%, about 80%, about 90%, about 92%, about 94%, or about 96% capture efficiency when capturing a population of rare cells (e.g., a population of TRLs). In some embodiments, the microfluidic device exhibits at most about 30%, about 40%, about 50%, about 60%, about 70%, about 80%, about 90%, about 92%, about 94%, about 96%, or about 98% capture efficiency when capturing a population of rare cells (e.g., a population of TRLs.

In some embodiments, the methods of isolating a population of TRLs described herein achieves a higher cell recovery than a standard cell-sorting approach (e.g., fluorescence activated cell sorting, or MACS) performed on the same sample. In some embodiments, the microfluidic approach to cell sorting described herein achieves a higher cell recovery than a standard cell-sorting approach, while retaining similar purity.

In some embodiments, the microfluidic approach to cell sorting described herein achieves a higher cell recovery by at least about 0.5 fold to about 50 fold as compared to a standard cell-sorting approach. In some embodiments, the microfluidic approach to cell sorting described herein achieves a higher cell recovery by about 0.5 fold to about 1 fold, about 0.5 fold to about 2 fold, about 0.5 fold to about 2.5 fold, about 0.5 fold to about 5 fold, about 0.5 fold to about 7.5 fold, about 0.5 fold to about 10 fold, about 0.5 fold to about 20 fold, about 0.5 fold to about 30 fold, about 0.5 fold to about 40 fold, about 0.5 fold to about 50 fold, about 1 fold to about 2 fold, about 1 fold to about 2.5 fold, about 1 fold to about 5 fold, about 1 fold to about 7.5 fold, about 1 fold to about 10 fold, about 1 fold to about 20 fold, about 1 fold to about 30 fold, about 1 fold to about 40 fold, about 1 fold to about 50 fold, about 2 fold to about 2.5 fold, about 2 fold to about 5 fold, about 2 fold to about 7.5 fold, about 2 fold to about 10 fold, about 2 fold to about 20 fold, about 2 fold to about 30 fold, about 2 fold to about 40 fold, about 2 fold to about 50 fold, about 2.5 fold to about 5 fold, about 2.5 fold to about 7.5 fold, about 2.5 fold to about 10 fold, about 2.5 fold to about 20 fold, about 2.5 fold to about 30 fold, about 2.5 fold to about 40 fold, about 2.5 fold to about 50 fold, about 5 fold to about 7.5 fold, about 5 fold to about 10 fold, about 5 fold to about 20 fold, about 5 fold to about 30 fold, about 5 fold to about 40 fold, about 5 fold to about 50 fold, about 7.5 fold to about 10 fold, about 7.5 fold to about 20 fold, about 7.5 fold to about 30 fold, about 7.5 fold to about 40 fold, about 7.5 fold to about 50 fold, about 10 fold to about 20 fold, about 10 fold to about 30 fold, about 10 fold to about 40 fold, about 10 fold to about 50 fold, about 20 fold to about 30 fold, about 20 fold to about 40 fold, about 20 fold to about 50 fold, about 30 fold to about 40 fold, about 30 fold to about 50 fold, or about 40 fold to about 50 fold as compared to a standard cell-sorting approach. In some embodiments, the microfluidic approach to cell sorting described herein achieves a higher cell recovery by about 0.5 fold, about 1 fold, about 2 fold, about 2.5 fold, about 5 fold, about 7.5 fold, about 10 fold, about 20 fold, about 30 fold, about 40 fold, or about 50 fold as compared to a standard cell-sorting approach. In some embodiments, the microfluidic approach to cell sorting described herein achieves a higher cell recovery by at least about 0.5 fold, about 1 fold, about 2 fold, about 2.5 fold, about 5 fold, about 7.5 fold, about 10 fold, about 20 fold, about 30 fold, or about 40 fold as compared to a standard cell-sorting approach. In some embodiments, the microfluidic approach to cell sorting described herein achieves a higher cell recovery by at most about 1 fold, about 2 fold, about 2.5 fold, about 5 fold, about 7.5 fold, about 10 fold, about 20 fold, about 30 fold, about 40 fold, or about 50 fold as compared to a standard cell-sorting approach.

In some embodiments, methods described herein may comprise isolating from a peripheral blood sample a population of CD103, CD39, SLC6A19, and/or SIDT1 expressing lymphocytes by treating the sample with a plurality of major histocompatibility complex (MHC) multimers mimicking a defined tumor epitope. Generally, T-cells express surface T-cell receptors (TCR) which enable T-cells to recognize peptide antigens bound to major histocompatibility complex (MHC) molecules, and TCR recognition of MHC-peptide complexes result in T-cell activation, clonal expansion and differentiation of the T-cells into effector, memory and regulatory T-cells. MHC-multimers comprise multiple copies of MHC-peptide complexes. In some embodiments, MHC multimers exhibit increased T-Cell affinity, compared to a monomer of the same complex. In some embodiments, the MHC molecules are human MHC molecules. In some embodiments, the MHC molecules are murine MHC molecules. In some embodiments, the MHC molecules are class 1 MHC molecules. In some embodiments, the MHC Class 1 molecules are human HLA-A, HLA-B, or HLA-C molecules. In some embodiments, the MHC class I molecules are murine H-2K, H-2D or H-2L molecules. In some embodiments, the molecules are class 2 MHC molecules. In some embodiments, an MHC multimer comprises at least 1, 2, 3, 4, 5, 7, 8, 9, 10, 11, 12, 13, 14, 15, 16, 17, 18, 19 or 20 MHC molecules. In some embodiments, an MHC multimer comprises about 1, 2, 3, 4, 5, 7, 8, 9, 10, 11, 12, 13, 14, 15, 16, 17, 18, 19 or 20 MHC molecules. In some embodiments, an MHC multimer comprises an MHC dimer comprising two MHC molecules. In some embodiments, an MHC multimer comprises an MHC tetramer comprising four MHC molecules. In some embodiments, an MHC multimer comprises an MHC pentamer comprising five MHC molecules. In some embodiments, an MHC multimer comprises a dextramer, comprising 10 or more MHC molecules.

In some embodiments, an MHC multimer may comprise a peptide. In some embodiments, an MHC multimer may comprise a peptide-MHC complex. In some embodiments, the peptide can be any natural or non-natural peptide capable of being presented by an MHC molecule. In some embodiments, the peptide-MHC complex can be one to which a TRL has reactivity. In some embodiments, the peptide-MHC complex can be one to which a CD8 and CD103 expressing lymphocyte has reactivity. In some embodiments, the peptide-MHC complex can be one to which a CD39 and CD103 expressing lymphocyte has reactivity. In some embodiments, the peptide-MHC complex mimics a defined tumor epitope. In some embodiments, the peptide comprises an epitope from influenza A hemagglutinin. In some embodiments, the peptide comprises amino acid residues 533-541 of influenza A hemagglutinin. In some embodiments, the peptide comprises an epitope from chicken ovalbumin. In some embodiments, the peptide comprises amino acid residues 257-264 of chicken ovalbumin. In some embodiments, an MHC multimer comprises a plurality of peptide-MHC complexes. In some embodiments, each of the peptide-MHC complexes is associated with a multimerization domain. In some instance, the peptide-MHC complex comprises the MC-38-derived peptide of SIIVFNLL sequence and an H-2Kb molecule. In some embodiments, the MHC multimer is operatively linked to a magnetic nanoparticle. In some embodiments, the magnetic nanoparticle is joined to the MHC multimer via fluorophore linker.

In some embodiments, the linker can comprise a polymer linker, such as an amino acid linker, biotin linker, and the like. In some embodiments, the linker can be cleavable. In some embodiments, the linker cannot be cleavable.

### Microfluidic Devices for Cell Sorting

In some embodiments, methods described herein can comprise separating from a peripheral blood sample a population of TRLs. In some embodiments, a microfluidic device disclosed herein can be used to separate the population of TRLs (e.g., CD103+ lymphocytes) from the peripheral blood sample. In some embodiments, isolated TRLs may comprise CD103+ lymphocytes. In some embodiments, the isolated CD103+ lymphocytes may be CD8+CD103+ lymphocytes, CD3+CD103+ lymphocytes, CD4+CD103+ lymphocytes, or CD39+CD103+ lymphocytes. In some embodiments, the isolated CD103+ lymphocytes may be CD8+CD39+CD103+ lymphocytes, CD3+CD39+CD103+ lymphocytes, or CD4+CD39+CD103+ lymphocytes. In some embodiments, the isolated CD103+ lymphocytes may comprise CD8+CD103+SLC6A19+ lymphocytes, CD3+CD103+SLC6A19+ lymphocytes, or CD4+CD103+SLC6A19+ lymphocytes. In some embodiments, the isolated CD103+ lymphocytes may comprise CD8+CD103+SLC6A19+SIDT1+ lymphocytes, CD3+CD103+SLC6A19+SIDT1+ lymphocytes, or CD4+CD103+SLC6A19+SIDT1+ lymphocytes.

In some embodiments, any of the microfluidic devices disclosed in PCT Publication No. WO 2014/166000, the contents of which are incorporated by reference herein, can be used to separate the TRLs from the peripheral blood sample. In some embodiments, once the TRLs have been separated, the TRLs may be eluted from the microfluidic device by removing an attractant acting on the TRLs (e.g., via removal of a magnetic field). In some embodiments, the captured TRLs can then be expanded, enhanced, or a combination thereof, by any of the methods disclosed herein.

Microfluidic devices disclosed herein can be configured to magnetically sort a population of cells. In some embodiments, the population of cells may comprise a population of TRLs. In some embodiments, the population of TRLs may be labeled with magnetic nanoparticles. In some embodiments, each of the magnetic nanoparticles can be about 0-50 nm in diameter, 51-100 nm in diameter, 100-150 nm in diameter, or 150-200 nm in diameter. In some embodiments, each of the magnetic nanoparticles can be about 50 nm in diameter. In some embodiments, compared with conventional microbeads, magnetic nanoparticles can have improved colloidal stability, which may be useful for processing larger samples. In some embodiments, cells labeled with magnetic nanoparticles may be difficult to capture because their orders-of-magnitude lower magnetic susceptibilities, compared to microbeads, result in lower capture efficiencies. Therefore, in some embodiments, the microfluidic devices disclosed herein can include flow rate-reducing structures that give rise to localized regions of lower flow rate, as a sample comprising the cells is flowed through the device. In some embodiments, the presence of such low flow velocity regions can enable capture of the magnetically labeled cells.

In some embodiments, a microfluidic device disclosed herein can comprise a microfluidic chip. In some embodiments, the microfluidic the chip can comprise a sorting chamber. In some embodiments, the sorting chamber can be etched or molded into the chip. In some embodiments, the sorting chamber can be in communication with a flow inlet and a flow outlet. In some embodiments, the flow inlet can be configured to receive a sample, e.g., a peripheral blood sample comprising a population of TRLs suspended in a fluid medium, and the outlet can be configured for delivering the fluid medium depleted of said TRLs. In some instance tubing can be connected to the inlet such that the fluid medium can be delivered into the inlet through the tubing. In some embodiments, tubing can be connected to the outlet such that the fluid medium can be received from the outlet through the tubing. In some embodiments, the tubing can be silicone tubing. In some embodiments, the microfluidic device can comprise a syringe pump capable of controlling the flow rate of fluid medium at the inlet.

In some embodiments, the sorting chamber can comprise at least one magnetic capture zone. In some embodiments, the sorting chamber can comprise a plurality of magnetic capture zones. In some embodiments, the sorting chamber can comprise at least 1, 2, 3, 4, 5, 6, 7, 8, 9, 10, 11, 12, 13, 14 or 15 magnetic capture zones. In some embodiments, the sorting chamber can comprise 1, 2, 3, 4, 5, 6, 7, 8, 9, 10, 11, 12, 13, 14 or 15 magnetic capture zones. In some embodiments, at least two of the magnetic capture zones can vary in height. In some embodiments, the magnetic capture zones can range from 50-800 μm in height. In some embodiments, sorting chamber can comprise three magnetic capture zones, one about 100 μm in height, one about 200 μm in height, and one about 400 μm in height.

In some embodiments, the microfluidic device can comprise at least one array of magnets positioned on the outer surface of the microfluidic chip, disposed above or below the sorting chamber, such that a magnetic field can be created in the magnetic capture zones by the at least one array of magnets. In some embodiments, the microfluidic device can comprise two arrays of magnetics positioned on two outer surfaces of the microfluidic chip above and below the sorting chamber such that a magnetic field is created in the magnetic capture zones by the two arrays of magnets. In some embodiments, magnets can be positioned in two arrays, with alternating polarities on opposing sides of the sorting chamber. In some embodiments, the at least one array of magnets can produce a magnetic field strength between 0.1-0.5 T, 0.5-1 T, or 1-1.5T in the magnetic capture zones. In some embodiments, the at least one array of magnets can produce a magnetic field strength between 0.5-1 T in the magnetic capture zones. In some embodiments, the magnets can comprise neodymium magnets. In some embodiments, the magnets can comprise N52 Nd FeB magnets.

In some embodiments, a magnetic capture zone can comprise a plurality of microstructures. In some embodiments, the microstructures can be flow rate-reducing structures configured to improve capture cells labeled with magnetic nanoparticles in the flow. In some embodiments, the microstructures can produce localized regions of lower flow rate, which may allow for capture of the particles (e.g., the reduced flow rate may allow the magnetic force to overcome the drag force on the particles). In some embodiments, the structures can be designed to avoid trapping of non-target particles. For example, despite being lower in flow rate, the regions of lower flow rate may still have enough flow velocity (that is, the flow rate may be at least non-zero) for non-target particles to be washed from the device, while target particles may be trapped in the low flow rate region. In some embodiments, the microstructures are X-shaped.

In some embodiments, the device can comprise a plurality of magnetic capture zones, with a first zone comprising the inlet of the sorting chamber, a final magnetic capture zone comprising the outlet of the sorting chamber, and a plurality of magnetic capture zones disposed between the first magnetic capture zone and the final magnetic capture zone. In some embodiments, the size or pattern of the microstructures can vary among the capture zones. In some embodiments, the heights can vary among the magnetic capture zones. In some embodiments, the first zone can exhibit the highest linear velocity and thus can retain cells with high magnetic content because the retaining magnetic force can overcome the drag force exerted by the locally high flow velocity. In some embodiments, the other magnetic capture zones can exhibit gradually reduced linear velocities, with the final magnetic capture zone exhibiting the lowest velocity. This design can allow cells with high levels of magnetization to be captured in the first zone of the device, whereas cells with lower magnetization can become sorted in later zones according to level of magnetization.

In some methods disclosed herein, a population of TRLs suspended in fluid can be propelled through the inlet, across the magnetic capture zones and through the outlet. In some embodiments, magnetically labelled cells can be captured in the capture zones if the magnetic force exerted on the cells is sufficient to overcome the drag force compelling the cell to flow through the capture zone. Otherwise, a cell would be flush into the next zone with a lower drag force, and eventually into the syringe if it cannot be captured by any zone. After sorting, captured cells at each zone can be recovered by removing the external magnets.

In some methods disclosed herein, a population of cells can be loaded into the microfluidic device, through the inlet, at a flow rate of at least 1 milliliters per hour, 3 mL h-1, 6 mL h-1, 9 mL h-1, 12 mL h-1, 15 mL h-1, 18 mL h-1,21 mL h-1.24 mL h-1, 27 mL h-1.30 mL h-1, 35 mL h-1, 40 mL h-1, 45 mL h-1, or 50 mL h-1. The magnetic force exerted on the cells can be determined by, for example, the size of the magnetic nanoparticle the number of magnetic nanoparticles attached to the cell, the size of the cell, and the strength of the applied magnetic field. In some embodiments, the relationship between drag force and linear flow velocity in a microfluidic device capable of magnetically capturing particles by leveraging rate reducing microstructures can be disclosed in PCT Publication No. WO 2014/166000, the contents of which are incorporated by reference herein.

In some embodiments, the microfluidic approaches to cell sorting disclosed herein can yield a population of CD39, CD103, SLC6A19, and/or SIDT1 expressing lymphocytes about 2 fold to about 20 fold higher than a population of CD39, CD103, SLC6A19, and/or SIDT1 expressing lymphocytes separated from a peripheral blood sample from the subject using fluorescence activated cell sorting (FACS). In some embodiments, the microfluidic approaches to cell sorting described herein can yield a population of CD39, CD103, SLC6A19, and/or SIDT1 expressing lymphocytes about 2 fold to about 4 fold, about 2 fold to about 7 fold, about 2 fold to about 8 fold, about 2 fold to about 10 fold, about 2 fold to about 12 fold, about 2 fold to about 14 fold, about 2 fold to about 16 fold, about 2 fold to about 17 fold, about 2 fold to about 18 fold, about 2 fold to about 19 fold, about 2 fold to about 20 fold, about 4 fold to about 7 fold, about 4 fold to about 8 fold, about 4 fold to about 10 fold, about 4 fold to about 12 fold, about 4 fold to about 14 fold, about 4 fold to about 16 fold, about 4 fold to about 17 fold, about 4 fold to about 18 fold, about 4 fold to about 19 fold, about 4 fold to about 20 fold, about 7 fold to about 8 fold, about 7 fold to about 10 fold, about 7 fold to about 12 fold, about 7 fold to about 14 fold, about 7 fold to about 16 fold, about 7 fold to about 17 fold, about 7 fold to about 18 fold, about 7 fold to about 19 fold, about 7 fold to about 20 fold, about 8 fold to about 10 fold, about 8 fold to about 12 fold, about 8 fold to about 14 fold, about 8 fold to about 16 fold, about 8 fold to about 17 fold, about 8 fold to about 18 fold, about 8 fold to about 19 fold, about 8 fold to about 20 fold, about 10 fold to about 12 fold, about 10 fold to about 14 fold, about 10 fold to about 16 fold, about 10 fold to about 17 fold, about 10 fold to about 18 fold, about 10 fold to about 19 fold, about 10 fold to about 20 fold, about 12 fold to about 14 fold, about 12 fold to about 16 fold, about 12 fold to about 17 fold, about 12 fold to about 18 fold, about 12 fold to about 19 fold, about 12 fold to about 20 fold, about 14 fold to about 16 fold, about 14 fold to about 17 fold, about 14 fold to about 18 fold, about 14 fold to about 19 fold, about 14 fold to about 20 fold, about 16 fold to about 17 fold, about 16 fold to about 18 fold, about 16 fold to about 19 fold, about 16 fold to about 20 fold, about 17 fold to about 18 fold, about 17 fold to about 19 fold, about 17 fold to about 20 fold, about 18 fold to about 19 fold, about 18 fold to about 20 fold, or about 19 fold to about 20 fold higher than a population of CD39, CD103, SLC6A19, and/or SIDT1 expressing lymphocytes separated from a peripheral blood sample from the subject using fluorescence activated cell sorting. In some embodiments, the microfluidic approaches to cell sorting described herein can yield a population of CD39, CD103, SLC6A19, and/or SIDT1 expressing lymphocytes about 2 fold, about 4 fold, about 7 fold, about 8 fold, about 10 fold, about 12 fold, about 14 fold, about 16 fold, about 17 fold, about 18 fold, about 19 fold, or about 20 fold higher than a population of CD39, CD103, SLC6A19, and/or SIDT1 expressing lymphocytes separated from a peripheral blood sample from the subject using fluorescence activated cell sorting. In some embodiments, the microfluidic approaches to cell sorting described herein can yield a population of CD39, CD103, SLC6A19, and/or SIDT1 expressing lymphocytes at least about 2 fold, about 4 fold, about 7 fold, about 8 fold, about 10 fold, about 12 fold, about 14 fold, about 16 fold, about 17 fold, about 18 fold, or about 19 fold higher than a population of CD39, CD103, SLC6A19, and/or SIDT1 expressing lymphocytes separated from a peripheral blood sample from the subject using fluorescence activated cell sorting. In some embodiments, the microfluidic approaches to cell sorting described herein can yield a population of CD39, CD103, SLC6A19, and/or SIDT1 expressing lymphocytes at most about 4 fold, about 7 fold, about 8 fold, about 10 fold, about 12 fold, about 14 fold, about 16 fold, about 17 fold, about 18 fold, about 19 fold, or about 20 fold higher than a population of CD39, CD103, SLC6A19, and/or SIDT1 expressing lymphocytes separated from a peripheral blood sample from the subject using fluorescence activated cell sorting.

In some embodiments, the microfluidic approaches to cell-sorting disclosed herein can achieve a percent recovery about 40% to about 99% of the CD39, CD103, SLC6A19, or SIDT1 lymphocytes. In some embodiments, the microfluidic approaches to cell-sorting disclosed herein can achieve a percent recovery about 40% to about 50%, about 40% to about 60%, about 40% to about 70%, about 40% to about 80%, about 40% to about 85%, about 40% to about 90%, about 40% to about 95%, about 40% to about 96%, about 40% to about 97%, about 40% to about 98%, about 40% to about 99%, about 50% to about 60%, about 50% to about 70%, about 50% to about 80%, about 50% to about 85%, about 50% to about 90%, about 50% to about 95%, about 50% to about 96%, about 50% to about 97%, about 50% to about 98%, about 50% to about 99%, about 60% to about 70%, about 60% to about 80%, about 60% to about 85%, about 60% to about 90%, about 60% to about 95%, about 60% to about 96%, about 60% to about 97%, about 60% to about 98%, about 60% to about 99%, about 70% to about 80%, about 70% to about 85%, about 70% to about 90%, about 70% to about 95%, about 70% to about 96%, about 70% to about 97%, about 70% to about 98%, about 70% to about 99%, about 80% to about 85%, about 80% to about 90%, about 80% to about 95%, about 80% to about 96%, about 80% to about 97%, about 80% to about 98%, about 80% to about 99%, about 85% to about 90%, about 85% to about 95%, about 85% to about 96%, about 85% to about 97%, about 85% to about 98%, about 85% to about 99%, about 90% to about 95%, about 90% to about 96%, about 90% to about 97%, about 90% to about 98%, about 90% to about 99%, about 95% to about 96%, about 95% to about 97%, about 95% to about 98%, about 95% to about 99%, about 96% to about 97%, about 96% to about 98%, about 96% to about 99%, about 97% to about 98%, about 97% to about 99%, or about 98% to about 99% of the 103+ lymphocytes. In some embodiments, the microfluidic approaches to cell-sorting disclosed herein can achieve a percent recovery about 40%, about 50%, about 60%, about 70%, about 80%, about 85%, about 90%, about 95%, about 96%, about 97%, about 98%, or about 99% of the CD39, CD103, SLC6A19, or SIDT1 lymphocytes. In some embodiments, the microfluidic approaches to cell-sorting disclosed herein can achieve a percent recovery at least about 40%, about 50%, about 60%, about 70%, about 80%, about 85%, about 90%, about 95%, about 96%, about 97%, or about 98% of the CD39, CD103, SLC6A19, or SIDT1 lymphocytes. In some embodiments, the microfluidic approaches to cell-sorting disclosed herein can achieve a percent recovery at most about 50%, about 60%, about 70%, about 80%, about 85%, about 90%, about 95%, about 96%, about 97%, about 98%, or about 99% of the CD39, CD103, SLC6A19, or SIDT1 lymphocytes.

In some embodiments, the purity of isolated cells using methods described herein can be about 10% to about 99% or essentially pure (e.g., 100%). In some embodiments, the purity of isolated cells using methods of described herein can be about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 10% to about 99%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 15% to about 99%, about 20% to about 25%, about 20% to about 30%, about 20% to about 40%, about 20% to about 50%, about 20% to about 60%, about 20% to about 70%, about 20% to about 80%, about 20% to about 90%, about 20% to about 99%, about 25% to about 30%, about 25% to about 40%, about 25% to about 50%, about 25% to about 60%, about 25% to about 70%, about 25% to about 80%, about 25% to about 90%, about 25% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99%. In some embodiments, the purity of isolated cells using methods of described herein can be about 10%, about 15%, about 20%, about 25%, about 30%, about 40%, about 50%, about 60%, about 70%, about 80%, about 90%, or about 99%. In some embodiments, the purity of isolated cells using methods of described herein can be at least about 10%, about 15%, about 20%, about 25%, about 30%, about 40%, about 50%, about 60%, about 70%, about 80%, or about 90%. In some embodiments, the purity of isolated cells using methods of described herein can be at most about 15%, about 20%, about 25%, about 30%, about 40%, about 50%, about 60%, about 70%, about 80%, about 90%, or about 99%.

### B. Culturing

Disclosed in some embodiments herein are methods of enriching and expanding a population of TRLs (e.g., cTRLs). In some embodiments, the methods can comprise magnetically separating a population of TRLs described using any of the methods disclosed herein and culturing the magnetically separated population of TRLs.

In some embodiments, an initial cell population comprising about 1,000 to about 20,000 magnetically separated CD103, CD39, SLC6A19, and/or SIDT1 expressing lymphocytes can be seed in the culture system described herein. In some embodiments, an initial cell population comprising about 1,000 to about 2,000, about 1,000 to about 3,000, about 1,000 to about 4,000, about 1,000 to about 5,000, about 1,000 to about 6,000, about 1,000 to about 7,000, about 1,000 to about 8,000, about 1,000 to about 9,000, about 1,000 to about 10,000, about 1,000 to about 15,000, about 1,000 to about 20,000, about 2,000 to about 3,000, about 2,000 to about 4,000, about 2,000 to about 5,000, about 2,000 to about 6,000, about 2,000 to about 7,000, about 2,000 to about 8,000, about 2,000 to about 9,000, about 2,000 to about 10,000, about 2,000 to about 15,000, about 2,000 to about 20,000, about 3,000 to about 4,000, about 3,000 to about 5,000, about 3,000 to about 6,000, about 3,000 to about 7,000, about 3,000 to about 8,000, about 3,000 to about 9,000, about 3,000 to about 10,000, about 3,000 to about 15,000, about 3,000 to about 20,000, about 4,000 to about 5,000, about 4,000 to about 6,000, about 4,000 to about 7,000, about 4,000 to about 8,000, about 4,000 to about 9,000, about 4,000 to about 10,000, about 4,000 to about 15,000, about 4,000 to about 20,000, about 5,000 to about 6,000, about 5,000 to about 7,000, about 5,000 to about 8,000, about 5,000 to about 9,000, about 5,000 to about 10,000, about 5,000 to about 15,000, about 5,000 to about 20,000, about 6,000 to about 7,000, about 6,000 to about 8,000, about 6,000 to about 9,000, about 6,000 to about 10,000, about 6,000 to about 15,000, about 6,000 to about 20,000, about 7,000 to about 8,000, about 7,000 to about 9,000, about 7,000 to about 10,000, about 7,000 to about 15,000, about 7,000 to about 20,000, about 8,000 to about 9,000, about 8,000 to about 10,000, about 8,000 to about 15,000, about 8,000 to about 20,000, about 9,000 to about 10,000, about 9,000 to about 15,000, about 9,000 to about 20,000, about 10,000 to about 15,000, about 10,000 to about 20,000, or about 15,000 to about 20,000 magnetically separated CD103, CD39, SLC6A19, and/or SIDT1 expressing lymphocytes can be seed in the culture system described herein. In some embodiments, an initial cell population comprising about 1,000, about 2,000, about 3,000, about 4,000, about 5,000, about 6,000, about 7,000, about 8,000, about 9,000, about 10,000, about 15,000, or about 20,000 magnetically separated CD103, CD39, SLC6A19, and/or SIDT1 expressing lymphocytes can be seed in the culture system described herein. In some embodiments, an initial cell population comprising at least about 1,000, about 2,000, about 3,000, about 4,000, about 5,000, about 6,000, about 7,000, about 8,000, about 9,000, about 10,000, or about 15,000 magnetically separated CD103, CD39, SLC6A19, and/or SIDT1 expressing lymphocytes can be seed in the culture system described herein. In some embodiments, an initial cell population comprising at most about 2,000, about 3,000, about 4,000, about 5,000, about 6,000, about 7,000, about 8,000, about 9,000, about 10,000, about 15,000, or about 20,000 magnetically separated CD103, CD39, SLC6A19, and/or SIDT1 expressing lymphocytes can be seed in the culture system described herein.

In some embodiments, culturing the magnetically separated CD103, CD39, SLC6A19, and/or SIDT1 expressing lymphocytes can comprise growing the CD103, CD39, SLC6A19, and/or SIDT1 expressing lymphocytes in a cell culture to expand the population of CD103, CD39, SLC6A19, and/or SIDT1 expressing lymphocytes. In some embodiments, the population of CD103, CD39, SLC6A19, and/or SIDT1 expressing lymphocytes can be expanded at least 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or at least 20-fold. In some embodiments, the population of CD103, CD39, SLC6A19, and/or SIDT1 expressing lymphocytes can be expanded at least 0-fold, 100-fold, 200-fold, 300-fold, 400-fold, 500-fold, 600-fold, 700-fold, 800-fold, 900-fold, 1000-fold, 1100-fold, 1200-fold, 1300-fold, 1400-fold, 1500-fold, 1600-fold, 1700-fold, 1800-fold, 1900-fold, 2000-fold, 2100-fold, 2200-fold, 2300-fold, 2400-fold, 2500-fold, 2600-fold, 2700-fold, 2800-fold, 2900-fold, 3000-fold, 3100-fold, 3200-fold, 3300-fold, 3400-fold, 3500-fold, 3600-fold, 3700-fold, 3800-fold, 3900-fold, 4000-fold, 4100-fold, 4200-fold, 4300-fold, 4400-fold, 4500-fold, 4600-fold, 4700-fold, 4800-fold, 4900-fold, or 5000-fold.

In some embodiments, the rapid expansion protocol disclosed in Dudley, M. E., Wunderlich, J. R., Shelton, T. E., Even, J. & Rosenberg, S. A., Generation of Tumor-Infiltrating Lymphocyte Cultures for Use in Adoptive Transfer Therapy for Melanoma Patients: J. Immunother. 26, 332-342 (2003), which is incorporated by reference in its entirety herein, can be used to culture the magnetically separated lymphocytes. In some embodiments, the culturing can be achieved in a culture flask or other container known in the art using feeder cells, T-Cell growth factors, and monoclonal antibodies capable of inducing T-Cell activation. In some embodiments, the culturing can be performed using a culture flask or container known to those of skill in the art. In some embodiments, culturing can be performed for 1, 2, 3, 4, 5, 6, 7, 8, 9, 10, 11, 11, 12, 13, or 14 days.

In some embodiments, the culture can be grown in the presence of culture medium. In some embodiments, the culture medium can comprise a T-Cell growth factor. In some embodiments, the T-Cell growth factor can include Il-2, Il-7, Il-9 or Il-15. In some embodiments, the T-Cell growth factor can comprise Il-2. In some embodiments, the culture medium can comprise at least about 6,000 IU/mL of IL-2. In some embodiments, the culture medium can comprise at least about 5-50 IU/mL, 50-500 IU/mL, 500-1000 IU/mL, 1000-1500 IU/mL, about 1500-2000 IU/mL, about 2000-2500 IU/mL, about 2500-3000 IU/mL, about 3000-3500 IU/mL, about 3500-4000 IU/mL, about 4000-4500 IU/mL, about 4500-5000 IU/mL, about 5000-5500 IU/mL, about 5500-6000 IU/mL, about 6000-6500 IU/mL, about 6500-7000 IU/mL, about 7000-7500 IU/mL, about 7500-8000 IU/mL or about 8000-8500 IU/mL IL-2.

In some embodiments, the culture medium can comprise an antibody (e.g., monoclonal antibody) capable of inducing T-Cell activation. In some embodiments, the culture medium can comprise an OKT-3 antibody. In some embodiments, the culture medium can comprise about 30 ng/mL of OKT-3 antibody. In some embodiments, the culture medium can comprise an antibody (e.g., monoclonal antibody) specific to CD2, CD3, CD28, or any combination thereof. In some embodiments, the culture medium can comprise a plurality of the antibodies disclosed herein, such as anti-OKT-3 antibodies, anti-CD2 antibodies, anti-CD3 antibodies, and anti-CD28 antibodies, or any combinations thereof. In some embodiments, the culture medium can be from about 0.1-5 ng/ml, about 5-10 ng/mL, about 10-15 ng/ml, about 15-20 ng/mL, about 20-25 ng/mL, about 25-30 ng/ml, about 30-35 ng/ml, about 35-40 ng/ml, about 40-45 ng/mL, or about 45-50 ng/ml of one or more, or each, the antibodies.

In some embodiments, the feeder cells can be allogeneic. In some embodiments, the feeder cells can be peripheral blood mononuclear cells (PBMCs). In some embodiments, the PBMCs can be obtained from standard whole blood from donors. In some embodiments, the ratio of TRL to feeder cells can be about 1:25, about 1:50, about 1:100, about 1:125, about 1:150, about 1:175, about 1:200, about 1:225, about 1:250, about 1:275, about 1:300, about 1:325, about 1:350, about 1:375 or about 1:400.

In some embodiments, TRL populations retrieved from the magnetic sorting methods described herein can be cultured using an expansion protocol describe herein. In some embodiments, irradiated PBMC feeder cells, OKT3 antibody, and the TRL can be combined, mixed, and aliquoted to a tissue culture flask or a tissue culture plate. In some embodiments, TRL populations can be cultured in a feeder-free system. In some embodiments, the flask can be incubated upright at 37° C. in 5% CO2. In some embodiments, IL-2 is added to the culture flask at 6000 IU/mL on day 2. In some embodiments, IL-2 can be added to the culture flask at a concentration ranging from 5 to 50 IU/mL. In some embodiments, IL-2 can be added to the culture flask at a concentration ranging from 5 to 6000 IU/mL. In some embodiments, on day 5, culture supernatant can be removed by aspiration and the culture media can be replaced with a 1:1 mixture of CM/AIM V containing 6000 IU/mL IL-2. In some embodiments, on day 6 and every day thereafter, cell concentration can be determined and cells ae split into additional flasks or transferred to culture bags with additional medium containing 6000 IU/mL IL-2 as needed to maintain cell densities around 1×10⁶ cells/mL. In some embodiments, about 14 days after initiation of the culture, cells can be harvested from culture bags. In some embodiments, harvesting can be accomplished using a Baxter/Fenwal continuous centrifuge cell harvester system. In some embodiments, the harvested cells can be washed in sodium chloride. In some embodiments, the cells can be resuspended in sodium chloride with human albumin. In some embodiments, the resulting cell populations can be suitable for administration to a subject in need thereof.

### C. Methods of Producing Enhanced TRLs Cells

Disclosed in some embodiments are methods of producing a population of enhanced TRLs (e.g., cTRLs). In some embodiments, the methods can comprise isolating a population of TRLs described using any of the methods disclosed herein, culturing the isolated TRLs using any of the methods disclosed herein, and introducing a cell from the cultured TRLs to a nucleotide that expresses a therapeutically enhancing polypeptide under conditions sufficient to produce the enhancing polypeptide by the cell. In some embodiments, the nucleotide can encode a chimeric antigen receptor (CAR). In some embodiments, methods described herein can comprise culturing the therapeutically enhanced cells using any of the culturing methods disclosed herein.

In some embodiments, at least one cell from a population of TRLs disclosed herein can be enhanced to express a CAR or an exogenous T cell receptor (TCR). In some embodiments, at least one cell from an enriched population of CD103, CD39, SLC6A19, and/or SIDT1 expressing lymphocytes disclosed herein can be used to produce a CAR-T cell. In some embodiments, at least 5 cells from an enriched population of CD103, CD39, SLC6A19, and/or SIDT1 expressing lymphocytes disclosed herein can be used to produce CAR-T cells. In some embodiments, at least 10 cells from an enriched population of CD103, CD39, SLC6A19, and/or SIDT1 expressing lymphocytes disclosed herein can be used to produce CAR-T cells. In some embodiments, at least 50 cells from an enriched population of CD103, CD39, SLC6A19, and/or SIDT1 expressing lymphocytes disclosed herein can be used to produce CAR-T cells. In some embodiments, at least 100 cells from an enriched population of CD103, CD39, SLC6A19, and/or SIDT1 expressing lymphocytes disclosed herein can be used to produce CAR-T cells. In some embodiments, at least 500 cells from an enriched population of CD103, CD39, SLC6A19, and/or SIDT1 expressing lymphocytes disclosed herein can be used to produce CAR-T cells. In some embodiments, at least 1,000 cells from an enriched population of CD103, CD39, SLC6A19, and/or SIDT1 expressing lymphocytes disclosed herein can be used to produce CAR-T cells. In some embodiments, at least 2,000 cells from an enriched population of CD103, CD39, SLC6A19, and/or SIDT1 expressing lymphocytes disclosed herein can be used to produce CAR-T cells. In some embodiments, at least 5,000 cells from an enriched population of CD103, CD39, SLC6A19, and/or SIDT1 expressing lymphocytes disclosed herein can be used to produce CAR-T cells. In some embodiments, at least 10,000 cells from an enriched population of CD103, CD39, SLC6A19, and/or SIDT1 expressing lymphocytes disclosed herein can be used to produce CAR-T cells. In some embodiments, at least 15,000 cells from an enriched population of CD103, CD39, SLC6A19, and/or SIDT1 expressing lymphocytes disclosed herein can be used to produce CAR-T cells. In some embodiments, at least 20,000 cells from an enriched population of CD103, CD39, SLC6A19, and/or SIDT1 expressing lymphocytes disclosed herein can be used to produce CAR-T cells. In some embodiments, at least 25,000 cells from an enriched population of CD103, CD39, SLC6A19, and/or SIDT1 expressing lymphocytes disclosed herein can be used to produce CAR-T cells.

In some embodiments, at least one cell from an enriched population of CD103, CD39, SLC6A19, and/or SIDT1 expressing lymphocytes having a purity of at least 90% disclosed herein can be used to produce a CAR-T cell. In some embodiments, at least 5 cells from an enriched population of CD103, CD39, SLC6A19, and/or SIDT1 expressing lymphocytes having a purity of at least 90% disclosed herein can be used to produce a CAR-T cell. In some embodiments, at least 10 cells from an enriched population of CD103, CD39, SLC6A19, and/or SIDT1 expressing lymphocytes having a purity of at least 90% disclosed herein can be used to produce a CAR-T cell. In some embodiments, at least 50 cells from an enriched population of CD103, CD39, SLC6A19, and/or SIDT1 expressing lymphocytes having a purity of at least 90% disclosed herein can be used to produce a CAR-T cell. some instances, at least 100 cells from an enriched population of CD103, CD39, SLC6A19, and/or SIDT1 expressing lymphocytes having a purity of at least 90% disclosed herein can be used to produce a CAR-T cell. In some embodiments, at least 500 cells from an enriched population of CD103, CD39, SLC6A19, and/or SIDT1 expressing lymphocytes having a purity of at least 90% disclosed herein can be used to produce a CAR-T cell. In some embodiments, at least 1,000 cells from an enriched population of CD103, CD39, SLC6A19, and/or SIDT1 expressing lymphocytes having a purity of at least 90% disclosed herein can be used to produce a CAR-T cell. In some embodiments, at least 2,000 cells from an enriched population of CD103, CD39, SLC6A19, and/or SIDT1 expressing lymphocytes having a purity of at least 90% disclosed herein can be used to produce a CAR-T cell. In some embodiments, at least 5,000 cells from an enriched population of CD103, CD39, SLC6A19, and/or SIDT1 expressing lymphocytes having a purity of at least 90% disclosed herein can be used to produce a CAR-T cell. In some embodiments, at least 10,000 cells from an enriched population of CD103, CD39, SLC6A19, and/or SIDT1 expressing lymphocytes having a purity of at least 90% disclosed herein can be used to produce a CAR-T cell. In some embodiments, at least 15,000 cells from an enriched population of CD103, CD39, SLC6A19, and/or SIDT1 expressing lymphocytes having a purity of at least 90% disclosed herein can be used to produce a CAR-T cell. In some embodiments, at least 20,000 cells from an enriched population of CD103, CD39, SLC6A19, and/or SIDT1 expressing lymphocytes having a purity of at least 90% disclosed herein can be used to produce a CAR-T cell. In some embodiments, at least 25,000 cells from an enriched population of CD103, CD39, SLC6A19, and/or SIDT1 expressing lymphocytes having a purity of at least 90% disclosed herein can be used to produce a CAR-T cell.

In some embodiments, at least one cell from an enriched population of CD103, CD39, SLC6A19, and/or SIDT1 expressing lymphocytes having a purity of at least 80% disclosed herein can be used to produce a CAR-T cell. In some embodiments, at least 5 cells from an enriched population of CD103, CD39, SLC6A19, and/or SIDT1 expressing lymphocytes having a purity of at least 80% disclosed herein can be used to produce a CAR-T cell. In some embodiments, at least 10 cells from an enriched population of CD103, CD39, SLC6A19, and/or SIDT1 expressing lymphocytes having a purity of at least 80% disclosed herein can be used to produce a CAR-T cell. In some embodiments, at least 50 cells from an enriched population of CD103, CD39, SLC6A19, and/or SIDT1 expressing lymphocytes having a purity of at least 80% disclosed herein can be used to produce a CAR-T cell. some instances, at least 100 cells from an enriched population of CD103, CD39, SLC6A19, and/or SIDT1 expressing lymphocytes having a purity of at least 80% disclosed herein can be used to produce a CAR-T cell. In some embodiments, at least 500 cells from an enriched population of CD103, CD39, SLC6A19, and/or SIDT1 expressing lymphocytes having a purity of at least 80% disclosed herein can be used to produce a CAR-T cell. In some embodiments, at least 1,000 cells from an enriched population of CD103, CD39, SLC6A19, and/or SIDT1 expressing lymphocytes having a purity of at least 80% disclosed herein can be used to produce a CAR-T cell. In some embodiments, at least 2,000 cells from an enriched population of CD103, CD39, SLC6A19, and/or SIDT1 expressing lymphocytes having a purity of at least 80% disclosed herein can be used to produce a CAR-T cell. In some embodiments, at least 5,000 cells from an enriched population of CD103, CD39, SLC6A19, and/or SIDT1 expressing lymphocytes having a purity of at least 80% disclosed herein can be used to produce a CAR-T cell. In some embodiments, at least 10,000 cells from an enriched population of CD103, CD39, SLC6A19, and/or SIDT1 expressing lymphocytes having a purity of at least 80% disclosed herein can be used to produce a CAR-T cell. In some embodiments, at least 15,000 cells from an enriched population of CD103, CD39, SLC6A19, and/or SIDT1 expressing lymphocytes having a purity of at least 80% disclosed herein can be used to produce a CAR-T cell. In some embodiments, at least 20,000 cells from an enriched population of CD103, CD39, SLC6A19, and/or SIDT1 expressing lymphocytes having a purity of at least 80% disclosed herein can be used to produce a CAR-T cell. In some embodiments, at least 25,000 cells from an enriched population of CD103, CD39, SLC6A19, and/or SIDT1 expressing lymphocytes having a purity of at least 80% disclosed herein can be used to produce a CAR-T cell.

In some embodiments, at least one cell from an enriched population of CD103, CD39, SLC6A19, and/or SIDT1 expressing lymphocytes having a purity of at least 70% disclosed herein can be used to produce a CAR-T cell. In some embodiments, at least 5 cells from an enriched population of CD103, CD39, SLC6A19, and/or SIDT1 expressing lymphocytes having a purity of at least 70% disclosed herein can be used to produce a CAR-T cell. In some embodiments, at least 10 cells from an enriched population of CD103, CD39, SLC6A19, and/or SIDT1 expressing lymphocytes having a purity of at least 70% disclosed herein can be used to produce a CAR-T cell. In some embodiments, at least 50 cells from an enriched population of CD103, CD39, SLC6A19, and/or SIDT1 expressing lymphocytes having a purity of at least 70% disclosed herein can be used to produce a CAR-T cell. some instances, at least 100 cells from an enriched population of CD103, CD39, SLC6A19, and/or SIDT1 expressing lymphocytes having a purity of at least 70% disclosed herein can be used to produce a CAR-T cell. In some embodiments, at least 500 cells from an enriched population of CD103, CD39, SLC6A19, and/or SIDT1 expressing lymphocytes having a purity of at least 70% disclosed herein can be used to produce a CAR-T cell. In some embodiments, at least 1,000 cells from an enriched population of CD103, CD39, SLC6A19, and/or SIDT1 expressing lymphocytes having a purity of at least 70% disclosed herein can be used to produce a CAR-T cell. In some embodiments, at least 2,000 cells from an enriched population of CD103, CD39, SLC6A19, and/or SIDT1 expressing lymphocytes having a purity of at least 70% disclosed herein can be used to produce a CAR-T cell. In some embodiments, at least 5,000 cells from an enriched population of CD103, CD39, SLC6A19, and/or SIDT1 expressing lymphocytes having a purity of at least 70% disclosed herein can be used to produce a CAR-T cell. In some embodiments, at least 10,000 cells from an enriched population of CD103, CD39, SLC6A19, and/or SIDT1 expressing lymphocytes having a purity of at least 70% disclosed herein can be used to produce a CAR-T cell. In some embodiments, at least 15,000 cells from an enriched population of CD103, CD39, SLC6A19, and/or SIDT1 expressing lymphocytes having a purity of at least 70% disclosed herein can be used to produce a CAR-T cell. In some embodiments, at least 20,000 cells from an enriched population of CD103, CD39, SLC6A19, and/or SIDT1 expressing lymphocytes having a purity of at least 70% disclosed herein can be used to produce a CAR-T cell. In some embodiments, at least 25,000 cells from an enriched population of CD103, CD39, SLC6A19, and/or SIDT1 expressing lymphocytes having a purity of at least 70% disclosed herein can be used to produce a CAR-T cell.

In some embodiments, at least one cell from an enriched population of CD103, CD39, SLC6A19, and/or SIDT1 expressing lymphocytes having a purity of at least 60% disclosed herein can be used to produce a CAR-T cell. In some embodiments, at least 5 cells from an enriched population of CD103, CD39, SLC6A19, and/or SIDT1 expressing lymphocytes having a purity of at least 60% disclosed herein can be used to produce a CAR-T cell. In some embodiments, at least 10 cells from an enriched population of CD103, CD39, SLC6A19, and/or SIDT1 expressing lymphocytes having a purity of at least 60% disclosed herein can be used to produce a CAR-T cell. In some embodiments, at least 50 cells from an enriched population of CD103, CD39, SLC6A19, and/or SIDT1 expressing lymphocytes having a purity of at least 60% disclosed herein can be used to produce a CAR-T cell. some instances, at least 100 cells from an enriched population of CD103, CD39, SLC6A19, and/or SIDT1 expressing lymphocytes having a purity of at least 60% disclosed herein can be used to produce a CAR-T cell. In some embodiments, at least 500 cells from an enriched population of CD103, CD39, SLC6A19, and/or SIDT1 expressing lymphocytes having a purity of at least 60% disclosed herein can be used to produce a CAR-T cell. In some embodiments, at least 1,000 cells from an enriched population of CD103, CD39, SLC6A19, and/or SIDT1 expressing lymphocytes having a purity of at least 60% disclosed herein can be used to produce a CAR-T cell. In some embodiments, at least 2,000 cells from an enriched population of CD103, CD39, SLC6A19, and/or SIDT1 expressing lymphocytes having a purity of at least 60% disclosed herein can be used to produce a CAR-T cell. In some embodiments, at least 5,000 cells from an enriched population of CD103, CD39, SLC6A19, and/or SIDT1 expressing lymphocytes having a purity of at least 60% disclosed herein can be used to produce a CAR-T cell. In some embodiments, at least 10,000 cells from an enriched population of CD103, CD39, SLC6A19, and/or SIDT1 expressing lymphocytes having a purity of at least 60% disclosed herein can be used to produce a CAR-T cell. In some embodiments, at least 15,000 cells from an enriched population of CD103, CD39, SLC6A19, and/or SIDT1 expressing lymphocytes having a purity of at least 60% disclosed herein can be used to produce a CAR-T cell. In some embodiments, at least 20,000 cells from an enriched population of CD103, CD39, SLC6A19, and/or SIDT1 expressing lymphocytes having a purity of at least 60% disclosed herein can be used to produce a CAR-T cell. In some embodiments, at least 25,000 cells from an enriched population of CD103, CD39, SLC6A19, and/or SIDT1 expressing lymphocytes having a purity of at least 60% disclosed herein can be used to produce a CAR-T cell.

In some embodiments, the TRLs described herein can be engineered to express a T-Cell Receptor (TCR) or a chimeric antigen receptor (CAR), wherein the cell exhibits an antitumor property. In some embodiments, a TRL can be transformed with the TCR or CAR and the at least part of the TCR or CAR can be expressed on the cell surface. In some embodiments, the TRLs may be transduced with a viral vector encoding a TCR or CAR. In some embodiments, the viral vector can be a retroviral vector. In some embodiments, the viral vector can be a lentiviral vector. In some such embodiments, the cell may stably express the TCR or CAR. In another embodiment, the TRLs can be transfected with a nucleic acid, e.g., mRNA, cDNA, DNA, encoding a TCR or a CAR. In some such embodiments, the cell may transiently express the TCR or the CAR. In one aspect, the antigen binding domain of the TCR or CAR comprises a murine (e.g., rat or mouse) antibody or antibody fragment.

In some embodiments, the TRLs can be engineered to express a chimeric antigen receptor (e.g., CART), wherein the cell (e.g., “CART”) exhibits an antitumor property. In some embodiments, the methods disclosed herein can comprise a recombinant DNA construct comprising sequences encoding a CAR, wherein the CAR comprises an antigen binding domain (e.g., antibody, antibody fragment) that binds to an antigen of interest (e.g., tumor antigen). The intracellular signaling domain can comprise a costimulatory signaling domain and/or a primary signaling domain, e.g., a zeta chain. The costimulatory signaling domain can refer to a portion of the CAR comprising at least a portion of the intracellular domain of a costimulatory molecule.

In some embodiments, the enhanced TRLs can be an allogenic immune effector cell lacking expression of a functional T cell receptor (TCR) and/or human leukocyte antigen (HLA), e.g., HLA class I and/or HLA class II. An enhanced TRL lacking a functional TCR can be, e.g., engineered such that it does not express any functional TCR on its surface, engineered such that it does not express one or more subunits that comprise a functional TCR or engineered such that it produces very little functional TCR on its surface. Alternatively, the TRL can express a substantially impaired TCR, e.g., by expression of mutated or truncated forms of one or more of the subunits of the TCR. The term “substantially impaired TCR” means that this TCR will not elicit an adverse immune reaction in a host. Such cells can be created through the use of one or more gene editing systems. An enhanced TRL described herein can be, e.g., engineered such that it does not express a functional HLA on its surface. For example, an enhanced TRL described herein, can be engineered such that cell surface expression HLA, e.g., HLA class 1 and/or HLA class II, is downregulated. Such cells can be created through the use of one or more gene editing systems as described herein. In embodiments, the gene editing system targets a sequence encoding a component of one or more HLA molecules. In embodiments, the gene editing system can target a sequence encoding a factor which affects the expression of one or more HLA molecules. In embodiments, the gene editing system can target a regulator of MHC class I expression, for example a sequence encoding beta-2 microglobulin (B2M). In embodiments, the gene editing system can target a sequence encoding a regulator of MHC class II molecule expression, for example, CIITA. In embodiments, gene editing systems can target both a regulator of MHC class I expression (for example, B2M) and a regulator of MHC class II molecule expression (e.g., CIITA), such that at least MHC class I molecule and at least one MHC class II molecule expression is downregulated. Modified T cells that lack expression of a functional TCR and/or HLA can be obtained by any suitable means, including a knockout or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

Provided herein are delivery systems (e.g., viral-based systems or non-viral based systems), in which a polynucleotide encoding a TCR, or a CAR disclosed herein can be inserted. Representative viral expression vectors can include, but are not limited to, the adenovirus-based vectors (e.g., the adenovirus-based Per. C6 system available from Crucell, Inc. (Leiden, The Netherlands)), lentivirus-based vectors (e.g., the lentiviral-based pLPI from Life Technologies (Carlsbad, Calif.)) and retroviral vectors (e.g., the pFB-ERV plus pCFB-EGSH), herpes viruses. In some embodiments, the viral vector can be a lentivirus vector. Vectors derived from retroviruses such as the lentivirus can be used to achieve long-term gene transfer. In some embodiments, the lentivirus can transduce non-proliferating cells. In some embodiments, the lentivirus can have a low immunogenicity. In some embodiments, a suitable vector can comprise an origin of replication functional in at least one organism, a promoter sequence, convenient restriction endonuclease sites, and one or more selectable markers. Other suitable vectors may include integrating expression vectors, which may randomly integrate into the host cell's DNA, or may include a recombination site to enable the specific recombination between the expression vector and the host cell's chromosome. Such integrating expression vectors may utilize the endogenous expression control sequences of the host cell's chromosomes to effect expression of the desired protein. Examples of vectors that integrate in a site specific manner can include, for example, components of the flp-in system from Invitrogen (Carlsbad, Calif.) (e.g., pcDNA™5/FRT), or the cre-lox system, such as can be found in the pExchange-6 Core Vectors from Stratagene (La Jolla, Calif). Examples of vectors that randomly integrate into host cell chromosomes can include, for example, pcDNA3.1 (when introduced in the absence of T-antigen) from Invitrogen (Carlsbad, Calif), and pCI or pFNIOA (ACT) FLEXI™ from Promega (Madison, Wis.). Additional promoter elements, e.g., enhancers, can regulate the frequency of transcriptional initiation. Non-limiting example of a suitable promoter can be the cytomegalovirus (CMV) promoter sequence, which can constitutively drive high levels of expression of any polynucleotide sequence operatively linked thereto. Another non-limiting example of a suitable promoter can include human elongation growth factor 1 alpha 1 (hEF1a1). In some embodiments, the vector construct comprising a CAR described herein can comprise hEF1a1 functional variants. In some embodiments, the vector may comprise a constitutive promoter sequences including, but not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. In some embodiments, inducible promoters can be used. Examples of inducible promoters include, but are not limited to a metallothionine promoter, a glucocorticoid promoter, a progesterone promoter, and a tetracycline promoter.

In some embodiments, the expression vector to be introduced into a cell can also contain either a selectable marker gene or a reporter gene or both to facilitate identification and selection of expressing cells from the population of cells sought to be transfected or infected through viral vectors. In some embodiments, the selectable marker can be carried on a separate piece of DNA and used in a co-transfection procedure. Useful selectable markers include, for example, antibiotic-resistance genes, such as neomycin resistance gene (neo) and ampicillin resistance gene and the like. In some embodiments, a truncated epidermal growth factor receptor (HERIt or HERIt-1) tag can be used as a selectable marker gene. Reporter genes can be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Suitable reporter genes include genes encoding luciferase, beta-galactosidase, chloramphenicol acetyl transferase, secreted alkaline phosphatase, or the green fluorescent protein gene.

In some embodiments, the polynucleotide encoding a TCR, or a CAR described herein can also be introduced into TRLs using a non-viral based delivery system, such as the “Sleeping Beauty (SB) Transposon System,” which refers a synthetic DNA transposon system for introducing DNA sequences into the chromosomes of vertebrates. The Sleeping Beauty transposon system is composed of a Sleeping Beauty (SB) transposase and a SB transposon. In some embodiments, the Sleeping Beauty transposon system can include the SB 11 transposon system, the SB100X transposon system, or the SB 110 transposon system.

In some embodiments, non-viral based delivery systems (e.g., delivery vehicle) can include lipid-based delivery systems, polymeric delivery systems, inorganic compound-based nanoparticles, or extracellular vesicle-based delivery. In some embodiments, an exemplary delivery vehicle can be a liposome. Lipid formulations can be used for the introduction of the polynucleotide into a host cell (in vitro, ex vivo, or in vivo). In some embodiments, the polynucleotide may be associated with a lipid. The polynucleotide associated with a lipid can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure.

In some embodiments, an exemplary delivery vehicle can be a lipid nanoparticle (LNP). In some embodiments, polynucleotides encoding the compositions disclosed herein can be incorporated into or associated with one or more LNPs. In some embodiments, the LNP can comprise 1,2-dioleoyl-3-trimethylammonium propane (DOTAP), dioleoylphosphatidylethanolamine (DOPE), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy (polyethylene glycol)-2000] (DSPE-PEG), 1,2-dimyristoyl-rac-glycero-3-methoxypolyethyleneglycol-2000 (DMG-PEG 2K), and 1,2 distearoyl-sn-glycero-3 phosphocholine (DSPC) and/or comprising of one or more molecules selected from polyethylenimine (PEI) and poly (lactic-co-glycolic acid) (PLGA), and N-Acetylgalactosamine (GaINAc). The LNP can comprise one or more of a structural lipid (e.g., DSPC), a PEG-conjugated lipid (CDM-PEG), a cationic lipid (MC3), cholesterol, and a targeting ligand (e.g., GaINAc). In some embodiments, a nanoparticle described herein can be a particle having a diameter of less than about 1000 nm. In some embodiments, nanoparticles can have a greatest dimension (e.g., diameter) of about 500 nm or less, or about 400 nm or less, or about 300 nm or less, or about 200 nm or less, or about 100 nm or less. In some embodiments, nanoparticles described herein can have a greatest dimension ranging between about 50 nm and about 150 nm, or between about 70 nm and about 130 nm, or between about 80 nm and about 120 nm, or between about 90 nm and about 110 nm. In some embodiments, the nanoparticles described herein can have a greatest dimension (e.g., a diameter) of about 100 nm.

In some embodiment, TRLs transduced with a nucleic acid encoding a TCR or a CAR can be expanded, e.g., by a method described herein. In some embodiment, the enhanced TRLs (e.g., TRLs transduced with a nucleic acid encoding a CAR) can be expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells can be expanded for a period of 4 to 9 days. In one embodiment, the cells can be expanded for a period of 8 days or less, e.g., 7, 6 or 5 days.

Conditions appropriate for T cell culture (e.g., enhanced TRLs) may include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

In one embodiment, the enhanced TRLs can be expanded in an appropriate media (e.g., media described herein) that includes one or more interleukin that result in at least a 200-fold (e.g., 200-fold, 250-fold, 300-fold, 350-fold) increase in cells over a 14-day expansion period, e.g., as measured by a method described herein such as flow cytometry. In one embodiment, the enhanced TRLs are expanded in the presence IL-15 and/or IL-7 (e.g., IL-15 and IL-7).

Once a CAR (e.g., enhanced TRL) is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. For example, western blot analysis of CAR expression in primary T cells can be used to detect the presence of monomers and dimers. In vitro expansion of the enhanced CAR cells following antigen stimulation can be measured by flow cytometry. Animal models can also be used to measure a CART activity. For example, imaging technologies can be used to evaluate specific trafficking and proliferation of CARS in tumor-bearing animal models.

### D. Methods of Treatment

Disclosed herein are methods of treating a disease or a condition in a subject by administering to the subject a composition or pharmaceutical formulation disclosed herein. In some embodiments, the composition comprises the TRLs (e.g., cTRLs) or enhanced TRLs (e.g., enhanced cTRLs) disclosed herein. In some embodiments, a plurality of the TRLs have been therapeutically enhanced to express a chimeric antigen receptor (CAR) having antigenic specificity for a cancer antigen, e.g., of any of the cancers disclosed herein. In some embodiments, the cells have been isolated from the peripheral blood of a subject using any of the methods disclosed herein. In some embodiments, cells are autologous to the subject. In some embodiments, the cells have been isolated from the peripheral blood of the subject using any of the methods of isolating TRLs disclosed herein. In some embodiments, the isolated TRLs are expanded using any of the methods disclosed herein. In some embodiments, autologous cells are preferred over allogeneic cells because of because of their inherent heterogeneity that maximizes the tumor-recognizing T cell receptors (TCRs) while minimizing off-tissue effects.

In some embodiments, the TRLs (e.g., enhanced TRLs) are injected into a body part of a subject (e.g., a vein, a marrow, etc. of a patient). In some embodiments, the cells are administered by intradermal, subcutaneous, intramuscular, intravenous, intraosseous, intraperitoneal, intrathecal, epidural, intracardiac, intraarticular, intracavernous, and/or intravitreal injection.

In some embodiments, administration of a populations of cells disclosed herein is by a single administration, at continuous intervals, or at distinct intervals, and can be readily determined by a person skilled in the art. In some embodiments, the subject is injected with a dose of the cells at least about 1, 2, 3, 4, 5, 6, 7, 8, 9, 10, or more times. In some embodiments, the subject is injected with a dose of the cells at most about 10, 9, 8, 7, 6, 5, 4, 3, 2, or 1 time. In some embodiments, the subject is injected with a dose of the cells at a frequency of at least once every 1, 2, 3, 4, 5, 6, 7, 8, 9, 10, 20, 30, 60, 90, 180, 360, or more days. In some embodiments, the subject is injected with a dose of the cells at a frequency of at most once every 360, 180, 90, 60, 30, 20, 10, 9, 8, 7, 6, 5, 4, 3, 2, or 1 day. In some embodiments, the subject is injected with a dose of at least about 1×10⁴ cells/kg, 1×10⁵ cells/kg, 1×10⁶ cells/kg, 1×10⁷ cells/kg, 1×10⁸ cells/kg, 1×10⁹ cells/kg, 1×10¹⁰ cells/kg, 1×10¹¹ cells/kg or more. In some embodiments, the subject may be injected with at most a dose of about at least about 1×10⁴ cells, 1×10⁵ cells, 1×10⁶ cells, 1×10⁷ cells, 1×10⁸ cells, 1×10⁹ cells, 1×10¹⁰ cells, 1×10¹¹ cells, 1×10¹² cells, or more.

In some embodiments, the cells may be useful in a variety of applications, including, but not limited to, immunotherapy to treat diseases and disorders. Diseases and disorders that can be treated using the cells of the present disclosure include, but are not limited to, inflammatory conditions, cancer, infectious diseases, autoimmune diseases, and neurodegenerative diseases. In some embodiments, the cell therapies disclosed herein are used to treat a cancer. In some embodiments, the cell therapy is used to treat a tumor that is not optimal source material for tumor-infiltrating lymphocyte (TIL) isolation. In some embodiments, a cell therapy described herein, is used to treat a tumor wherein large resectable lesion (e.g., less than 3 cm in diameter) is not readily accessible. In some embodiments, a cell therapy described herein is used to treat a patient for which excisional surgery is not an option for patients (e.g., due to a substantial risk or rapid tumor progression). In some embodiments, a cell therapy described herein is used to treat a tumor which exhibited a reduced response rate to a TIL-based adoptive cell therapy. In some embodiments, the cell therapy is used to treat solid tumors, such as renal carcinoma, cervical cancer, and breast cancer, which have been demonstrated, in some embodiments, to be less responsive to TIL therapy. In some embodiments, the cells of the present disclosure are used to treat a cancer. In some embodiments, the cancer is in a tissue. In some embodiments, the tissue comprises a bone tissue, a muscle tissue, a breast tissue, an epithelial tissue, a connective tissue, a brain tissue, a lung tissue, a kidney tissue, a liver tissue, a pancreatic tissue, a prostate tissue, a lymphoid tissue, a myeloid tissue, or a bladder tissue. Non-limiting examples of cancer include: Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof.

In some embodiments, compositions and methods disclosed herein are administered in combination with other cancer immunotherapies or with a chemotherapy. In some embodiments, a method of providing a cell therapy to a subject in need thereof further comprises administering at least one additional therapy. In some embodiments, administering at least one additional therapy comprises administering to the subject an additional therapeutic agent. In some embodiments, the additional therapeutic agent can be an immunosuppressive drug. In some embodiments, the immunosuppressive drugs can comprise a monoclonal antibody. In some embodiments, the monoclonal antibody can deplete endogenous lymphocytes. In some embodiments, anti-CD3, anti-CD2, and/or anti-CD52 can be used to deplete endogenous lymphocytes. In some embodiments, the additional therapeutic agent is an anti-oncologic agent. In some embodiments, the anti-oncologic agent is a chemotherapeutic agent. In some embodiments, the chemotherapeutic agent is a lymphocyte depleting chemotherapeutic agent. In some embodiments, the anti-oncologic agent is an immunotherapy agent. In some embodiments, the anti-oncologic agent is an immune checkpoint inhibitor, a chemotherapeutic agent, or any combination thereof. In some embodiments, the anti-oncologic agent is a costimulatory molecule. In some embodiments, the costimulatory molecule is Glucocorticoid-Induced Tumor Necrosis Factor Receptor (GITR). In some embodiments, the costimulatory molecule can comprise CD28, CD137 (4-1BB), CD134 (OX40), Inducible T cell costimulatory (ICOS), CD27 or any combination thereof. In some embodiments, the ani-oncologic agent can be a cytokine that stimulates survival, proliferation and activation. In some embodiment, the cytokine can be IL-2, IL-7, IL-15, IL-21, or any combination thereof. In some embodiments, the anti-oncologic agent is an immune checkpoint inhibitor. In some embodiments, the immune checkpoint inhibitor is an anti-PD1 antibody, an anti-PD-L1 antibody, or a variant or functional fragment thereof. In some embodiments, the anti-PD1 antibody is selected from pidilizumab, BMS-936559, nivolumab, pembrolizumab, or a variant or functional fragment thereof. In some embodiments, the anti-PD-L1 antibody is selected from atezolizumab, avelumab, durvalumab, MDX-1105, or a variant or functional fragment thereof.

In some embodiments, the immune checkpoint inhibitor is an anti-CTLA-4 antibody, or a variant or functional fragment thereof. Anti-CTLA-4 antibodies of the present invention can bind human CTLA-4 to interfere with the interaction between CTLA-4 and human B7 receptors. Since the interaction between CTLA-4 and B7 transforms a signal leading to the inactivation of T-cells carrying the CTLA-4 receptor, interfering with the interaction effectively induces the activation of these T cells. An exemplary clinical anti-CTLA-4 Ab is human mAb 10D1 (now known as ipilimumab and marketed as YERVOY®). In some embodiments, the anti-CTLA-4 Ab is a mAb. In certain other embodiments, the anti-CTLA-4 antibody is a chimeric, humanized or human antibody. In an embodiment, the anti-CTLA-4 antibody is ipilimumab.

In some embodiments, the additional therapeutic agent is administered before administration of the cell population. For example, anti-CD3 can be administered before administration of the TRLs (e.g., enhanced TRLs) to deplete endogenous lymphocytes. In some embodiments, the additional therapeutic agent is administered after administration of the cell population. For example, IL-2 can be administered after administration of the TRLs (e.g., enhanced TRLs) to stimulate expansion (e.g., cell proliferation, activation, survival) of the administered TRLs. In some embodiments, the additional therapeutic agent is administered concurrently with administration of the cell population. In some embodiments, in some embodiments, the additional therapeutic agent is administered 1, 2, 3, 4, 5, 6, 7, 8, 9, 10, or more times. In some embodiments, the additional therapeutic agent is administered at most about 10, 9, 8, 7, 6, 5, 4, 3, 2, or 1 time. In some embodiments, the additional therapeutic agent is administered at a frequency of at least once every 1, 2, 3, 4, 5, 6, 7, 8, 9, 10, 20, 30, 60, 90, 180, 360, or more days. In some embodiments, the additional therapeutic agent is administered at a frequency of at most once every 360, 180, 90, 60, 30, 20, 10, 9, 8, 7, 6, 5, 4, 3, 2, or 1 day.

In practicing the methods of treatment or use provided herein, therapeutically effective amounts of pharmaceutical formulations described herein are administered to a mammal having a disease, disorder, or condition to be treated, e.g., cancer. In some embodiments, the mammal is a human. A therapeutically effective amount may vary widely depending on the severity of the disease, the age and relative health of the subject, the potency of the therapeutic agent used and other factors. In some embodiments, pharmaceutical formulations described herein, may be used singly or in combination with one or more therapeutic agents as components of mixtures.

### III. Systems

Described herein, in some embodiments, are systems comprising one or more compositions or devices disclosed herein. In some embodiments, the system comprises a microfluidic device and instructions for how to use the microfluidic device to magnetically separate to separate tumor reactive lymphocytes (TRLs) from a fluid sample from a subject. In some embodiments, the microfluid device comprises a magnetic capture zone disposed in a channel of the microfluidic device, wherein the microfluidic device is configured to magnetically separate the population of cells from the fluid sample. In some embodiments, the system comprises the fluid sample obtained from a subject, wherein the fluid sample comprises a population of cells comprising (i) CD103 lymphocytes, (ii) CD39 lymphocytes, (iii) SLC6A19+ lymphocytes, (iv) SIDT1+ lymphocytes, or (v) any combination of (i) to (iv). In some embodiments, the system comprises an enhanced TRL provided in Section I (Compositions) of this disclosure. In some embodiments, the system comprises a kit provided in Section IV (Kits) of this disclosure.

In some embodiments, the system may comprise a population of isolated and enriched tumor-reactive lymphocytes derived from a sample (e.g., peripheral blood) that express SLC6A19, SIDT1, CD103, CD39, or any combination of thereof. In some embodiments, a TRL expresses CD103. In some embodiments, a population of TRLs comprises a CD103 signature. In some embodiments, a CD103 signature defines the population of TRLs. In some embodiments, a TRL expresses CD39. In some embodiments, a population of TRLs comprises a CD39 signature. In some embodiments, a CD39 signature defines the population of TRLs. In some embodiments, a TRL expresses SLC6A19, SIDT1, or a combination thereof. In some embodiments, a population of TRLs comprise a SLC6A19+, SIDT1+ signature. In some embodiments, the population of TRLs can further comprise CD3, CD4, CD8 or any combination thereof. For example, the isolated and enriched TRLs can be CD3 and CD103 cells. In some embodiments, the isolated and enriched TRLs can be CD4 and CD103 cells. In some embodiments, the isolated and enriched TRLs can be CD8 and CD103 cells. In some embodiments, the isolated and enriched TRLs can be CD39 and CD103 cells. In some embodiments, the isolated and enriched TRLs can be CD3, CD39, and CD103 cells. In some embodiments, the isolated and enriched TRLs can be CD4, CD39, and CD103 cells. In some embodiments, the isolated and enriched TRLs can be CD8, CD39, and CD103 cells. In some embodiments, the isolated and enriched TRLs can be CD3, SLC6A19, and SIDT1 cells. In some embodiments, the isolated and enriched TRLs can be CD4, SLC6A19, and SIDT1 cells. In some embodiments, the isolated and enriched TRLs can be CD8, SLC6A19, and SIDT1 cells. In some embodiments, the isolated and enriched TRLs can be CD3, CD103, and SLC6A19 cells. In some embodiments, the isolated and enriched TRLs can be CD4, CD103, and SLC6A19 cells. In some embodiments, the isolated and enriched TRLs can be CD8, CD103, and SLC6A19 cells. In some embodiments, the isolated and enriched TRLs can be CD3, CD103, SLC6A19, and SIDT1 cells. In some embodiments, the isolated and enriched TRLs can be CD4, CD103, SLC6A19, and SIDT1 cells. In some embodiments, the isolated and enriched TRLs can be CD8, CD103, SLC6A19, and SIDT1 cells.

In some embodiments, a population of tumor-reactive lymphocytes (TRLs) may be found in a fluid sample of a patient having cancer. In some embodiments, a fluid sample may comprise a peripheral blood sample. In some embodiments, a fluid sample may be other biofluids, such as pleural effusion, ascites, and the like. In some embodiments, a fluid sample also be cord blood, bone marrow, lymph nodes, liver pleural effusion, thorax, abdominal cavity, synvial fluid, peritoneum, retroperitoneal space, thymus, and tumor.

In some embodiments, a system described herein comprises isolating or enriching a population of CD103, CD39, SLC6A19, and/or SIDT1 expressing lymphocytes from a peripheral blood sample by treating the sample with a plurality of major histocompatibility complex (MHC) multimers mimicking a defined tumor epitope. Generally, T-cells express surface T-cell receptors (TCR) which enable T-cells to recognize peptide antigens bound to major histocompatibility complex (MHC) molecules, and TCR recognition of MHC-peptide complexes result in T-cell activation, clonal expansion and differentiation of the T-cells into effector, memory and regulatory T-cells. MHC-multimers comprise multiple copies of MHC-peptide complexes. In some embodiments, MHC multimers exhibit increased T-Cell affinity, compared to a monomer of the same complex. In some embodiments, the MHC molecules are human MHC molecules. In some embodiments, the MHC molecules are murine MHC molecules. In some embodiments, the MHC molecules are class 1 MHC molecules. In some embodiments, the MHC Class 1 molecules are human HLA-A, HLA-B, or HLA-C molecules. In some embodiments, the MHC class I molecules are murine H-2K, H-2D or H-2L molecules. In some embodiments, the molecules are class 2 MHC molecules. In some embodiments, an MHC multimer comprises at least 1, 2, 3, 4, 5, 7, 8, 9, 10, 11, 12, 13, 14, 15, 16, 17, 18, 19 or 20 MHC molecules. In some embodiments, an MHC multimer comprises about 1, 2, 3, 4, 5, 7, 8, 9, 10, 11, 12, 13, 14, 15, 16, 17, 18, 19 or 20 MHC molecules. In some embodiments, an MHC multimer comprises an MHC dimer comprising two MHC molecules. In some embodiments, an MHC multimer comprises an MHC tetramer comprising four MHC molecules. In some embodiments, an MHC multimer comprises an MHC pentamer comprising five MHC molecules. In some embodiments, an MHC multimer comprises a dextramer, comprising 10 or more MHC molecules.

In some embodiments, an MHC multimer comprises a peptide. In some embodiments, an MHC multimer comprises a peptide-MHC complex. In some embodiments, the peptide is any natural or non-natural peptide capable of being presented by an MHC molecule. In some embodiments, the peptide-MHC complex is one to which a TRL has reactivity. In some embodiments, the peptide-MHC complex is one to which a CD8+CD103+ lymphocyte has reactivity. In some embodiments, the peptide-MHC complex is one to which a CD103+CD39+ lymphocyte has reactivity. In some embodiments, the peptide-MHC complex is one to which a CD8+CD103+CD39+ lymphocyte has reactivity. In some embodiments, the peptide-MHC complex mimics a defined tumor epitope. In some embodiments, the peptide comprises an epitope from influenza A hemagglutinin. In some embodiments, the peptide comprises amino acid residues 533-541 of influenza A hemagglutinin. In some embodiments, the peptide comprises an epitope from chicken ovalbumin. In some embodiments, the peptide comprises amino acid residues 257-264 of chicken ovalbumin. In some embodiments, an MHC multimer comprises a plurality of peptide-MHC complexes. In some embodiments, each of the peptide-MHC complexes is associated with a multimerization domain. In some instance, the peptide-MHC complex comprises the MC-38-derived peptide of SIIVFNLL sequence and an H-2Kb molecule. In some embodiments, the MHC multimer is operatively linked to a magnetic nanoparticle. In some embodiments, the magnetic nanoparticle is joined to the MHC multimer via fluorophore linker.

In some embodiments, the linker comprises a polymer linker, such as an amino acid linker, biotin linker, and the like. In some embodiments, the linker is cleavable. In some embodiments, the linker is not cleavable.

In some embodiments, a system described herein may comprise separating, isolating or enriching from a peripheral blood sample a population of TRLs. In some embodiments, a microfluidic device disclosed herein is used to separate the population of TRLs from the peripheral blood sample. In some embodiments, any of the microfluidic devices disclosed in PCT Publication No. WO 2014/166000, the contents of which are incorporated by reference herein, are used to separate the TRLs from the peripheral blood sample. In some embodiments, once the TRLs have been separated, the TRLs may be eluted from the microfluidic device by removing an attractant acting on the TRLs (e.g., via removal of a magnetic field). In some embodiments, the captured TRLs are then be expanded, enhanced, or a combination thereof, by any of the methods disclosed herein.

Microfluidic devices disclosed herein are configured to magnetically sort a population of cells. In some embodiments, the microfluidic device is provided in U.S. Pat. No. 10,073,079, which is hereby incorporated by reference in its entirety. In some embodiments, the population of cells comprises a population of TRLs. In some embodiments, the population of TRLs have been labeled with magnetic nanoparticles. In some embodiments, each of the magnetic nanoparticles are about 0-50 nanometer (nm) in diameter, 51-100 nm in diameter, 100-150 nm in diameter, or 150-200 nm in diameter. In some embodiments, each of the magnetic nanoparticles are about 50 nm in diameter. In some embodiments, compared with conventional microbeads, magnetic nanoparticles have improved colloidal stability, which may be useful for processing larger samples. In some embodiments, cells labeled with magnetic nanoparticles are difficult to capture because their orders-of-magnitude lower magnetic susceptibilities, compared to microbeads, result in lower capture efficiencies. Therefore, in some embodiments, the microfluidic devices disclosed herein include flow rate-reducing structures that give rise to localized regions of lower flow rate, as a sample comprising the cells is flowed through the device. In some embodiments, the presence of such low flow velocity regions enables capture of the magnetically labeled cells

In some embodiments, a microfluidic device disclosed herein comprises a microfluidic chip. In some embodiments, the microfluidic the chip comprises a sorting chamber. In some embodiments, the sorting chamber is etched or molded into the chip. In some embodiments, the sorting chamber is in communication with a flow inlet and a flow outlet. In some embodiments, the flow inlet is configured to receive a sample, e.g., a peripheral blood sample comprising a population of TRLs suspended in a fluid medium, and the outlet is configured for delivering the fluid medium depleted of said TRLs. In some instance tubing is connected to the inlet such that the fluid medium can be delivered into the inlet through the tubing. In some embodiments, tubing is connected to the outlet such that the fluid medium can be received from the outlet through the tubing. In some embodiments, the tubing is silicone tubing. In some embodiments, the microfluidic device comprises a syringe pump capable of controlling the flow rate of fluid medium at the inlet.

In some embodiments, the sorting chamber comprises at least one magnetic capture zone. In some embodiments, the sorting chamber comprises a plurality of magnetic capture zones. In some embodiments, the sorting chamber comprises at least 1, 2, 3, 4, 5, 6, 7, 8, 9, 10, 11, 12, 13, 14 or 15 magnetic capture zones. In some embodiments, the sorting chamber comprises 1, 2, 3, 4, 5, 6, 7, 8, 9, 10, 11, 12, 13, 14 or 15 magnetic capture zones. In some embodiments, at least two of the magnetic capture zones vary in height. In some embodiments, the magnetic capture zones range from 50-800 μm in height. In some embodiments, sorting chamber comprises three magnetic capture zones, one about 100 μm in height, one about 200 μm in height, and one about 400 μm in height.

In some embodiments, the microfluidic device comprises at least one array of magnets positioned on the outer surface of the microfluidic chip, disposed above or below the sorting chamber, such that a magnetic field is created in the magnetic capture zones by the at least one array of magnets. In some embodiments, the microfluidic device comprises two arrays of magnetics positioned on two outer surfaces of the microfluidic chip above and below the sorting chamber such that a magnetic field is created in the magnetic capture zones by the two arrays of magnets. In some embodiments, magnets are positioned in two arrays, with alternating polarities on opposing sides of the sorting chamber. In some embodiments, the at least one array of magnets produce a magnetic field strength between 0.1-0.5 Tesla (T), 0.5-1 T, or 1-1.5T in the magnetic capture zones. In some embodiments, the at least one array of magnets produce a magnetic field strength between 0.5-1 T in the magnetic capture zones. In some embodiments, the magnets comprise neodymium magnets. In some embodiments, the magnets comprise N52 Nd FeB magnets.

In some embodiments, a magnetic capture zone comprises a plurality of microstructures. In some embodiments, the microstructures are flow rate-reducing structures configured to improve capture cells labeled with magnetic nanoparticles in the flow. In some embodiments, the microstructures produce localized regions of lower flow rate, which may allow for capture of the particles (e.g., the reduced flow rate may allow the magnetic force to overcome the drag force on the particles). In some embodiments, the structures are designed to avoid trapping of non-target particles. For example, despite being lower in flow rate, the regions of lower flow rate may still have enough flow velocity (that is, the flow rate may be at least non-zero) for non-target particles to be washed from the device, while target particles may be trapped in the low flow rate region. In some embodiments, the microstructures are X-shaped.

In some embodiments, the device comprises a plurality of magnetic capture zones, with a first zone comprising the inlet of the sorting chamber, a final magnetic capture zone comprising the outlet of the sorting chamber, and a plurality of magnetic capture zones disposed between the first magnetic capture zone and the final magnetic capture zone. In some embodiments, the size or pattern of the microstructures varies among the capture zones. In some embodiments, the heights vary among the magnetic capture zones. In some embodiments, the first zone exhibits the highest linear velocity and thus retains cells with high magnetic content because the retaining magnetic force overcomes the drag force exerted by the locally high flow velocity. In some embodiments, the other magnetic capture zones exhibit gradually reduced linear velocities, with the final magnetic capture zone exhibiting the lowest velocity. This design allows cells with high levels of magnetization to be captured in the first zone of the device, whereas cells with lower magnetization become sorted in later zones according to level of magnetization.

In some methods disclosed herein, a population of TRLs suspended in fluid are propelled through the inlet, across the magnetic capture zones and through the outlet. In some embodiments, magnetically labelled cells are captured in the capture zones if the magnetic force exerted on the cells is sufficient to overcome the drag force compelling the cell to flow through the capture zone. Otherwise, a cell would be flush into the next zone with a lower drag force, and eventually into the syringe if it cannot be captured by any zone. After sorting, captured cells at each zone are recovered by removing the external magnets.

In some methods disclosed herein, a population of cells are loaded into the microfluidic device, through the inlet, at a flow rate of at least 1 milliliter per hour (mL h-1), 3 mL h-1, 6 mL h-1, 9 mL h-1, 12 mL h-1, 15 mL h-1, 18 mL h-1, 21 mL h-1, 24 mL h-1, 27 mL h-1, 30 mL h-1, 35 mL h-1, 40 mL h-1, 45 mL h-1, or 50 mL h-1. The magnetic force exerted on the cells will be determined by, for example, the size of the magnetic nanoparticle the number of magnetic nanoparticles attached to the cell, the size of the cell, and the strength of the applied magnetic field. In some embodiments, the relationship between drag force and linear flow velocity in a microfluidic device capable of magnetically capturing particles by leveraging rate reducing microstructures is disclosed in U.S. Pat. No. 10,073,079, which is hereby incorporated by reference in its entirety.

Disclosed in some embodiments herein are systems of producing a population of enhanced TRLs. In some embodiments, the systems comprise isolating a population of TRLs described using any of the systems disclosed herein, culturing the isolated TRLs using any of the systems disclosed herein, and introducing a cell from the cultured TRLs to a nucleotide that expresses a therapeutically enhancing polypeptide under conditions sufficient to produce the enhancing polypeptide by the cell. In some embodiments, the nucleotide encodes a chimeric antigen receptor (CAR). In some embodiments, systems of the present inventive concepts comprise culturing the therapeutically enhanced cells using any of the culturing systems disclosed herein.

Described herein are pharmaceutical formulations comprising the enhanced TRLs or the compositions described herein. In some embodiments, the pharmaceutical formulations further comprise a pharmaceutically acceptable: carrier, excipient, diluent, or nebulized inhalant.

In some embodiments, the pharmaceutical formulations include two or more active agents, or two or more therapeutic agents as disclosed herein. In some embodiments, the two or more active agents are contained in a single dosage unit such as, for example, when the enhanced TRLs (e.g., CAR or TCR) comprises two or more therapeutic agents. In embodiments, the two or more active agents are contained in separate dosage units such as when the enhanced TRLs (e.g., CAR or TCR) is administered separately from an additional therapeutic agent or adjuvant. In some embodiments, the active agents that may be, in some embodiments, the additional therapeutic agent include a chemotherapeutic agent, cytotoxic agent, cytokine, growth-inhibitory agent, anti-hormonal agent, anti-angiogenic agent, cardio protectant, and/or checkpoint inhibitor.

### IV. Kits

Disclosed herein, in some embodiments, are kits comprising the compositions or systems disclosed herein, with instructions for how to produce or use the compositions or systems. In some embodiments, the kit comprises the microfluidic device disclosed herein, with instructions for how to use the microfluidic device to isolate TRL's from a fluid sample obtained from a subject. In some embodiments, the kit further comprises reagents for isolating, enriching or expanding the TRLs from the fluid sample. Such reagents may include antibodies (e.g., magnetic nanoparticles), buffers, and/or conditioned medium.

In some embodiments, the kit comprises a vector comprising polynucleotides encoding the CAR components described herein. The kit can include multiple vectors each encoding different proteins or subsets of proteins. These vectors can be viral, non-viral, episomal, or integrating. In some embodiments, the vectors are transposons. In some embodiments, the kit further comprises reagents or devices for TRL transfection or transduction, such as calcium phosphate DNA co-precipitation, DEAE-dextran, electroporation, cationic liposome-mediated transfection, tungsten particle-facilitated microparticle bombardment, and strontium phosphate DNA co-precipitation.

In some embodiments, the instructions further comprise instructions for how to engineer the TRLs to produce enhanced TRLs. Instructions may also include instructions for cryopreserving, lyophilizing, or cryo-hibernating the compositions disclosed herein during storage and/or transport. Instructions may also include instructions for thawing or otherwise reviving the biological activity of the compositions disclosed herein prior to administration to a subject. In some embodiments, the instructions may direct a medical laboratory to separate TRLs from a fluid sample disclosed herein (e.g., peripheral blood) utilizing the system components disclosed herein (e.g., microfluidic device). For example, the instructions may include methods comprising separating from a fluid sample of a subject a population of SLC6A19+SIDT1+ lymphocytes. In some embodiments, the instructions may include methods comprising magnetically separating from a fluid sample of a subject a population of CD103 lymphocytes, wherein the magnetically separating comprises flowing the CD103 lymphocytes across a magnetic capture zone disposed in a channel of a microfluidic device. In some embodiments, instructions comprise methods for administering to the subject the pharmaceutical formulation disclosed herein or the engineered TRL disclosed herein. In some embodiments, the methods further comprise treating a cancer in the subject with the pharmaceutical formulation or engineered TRL disclosed herein. Generally, the instructions may comprise any of the methods disclosed herein.

In some embodiments, the kits disclosed herein may be used to treat a disease or condition in a subject. For example, the kit may comprise a population of TRLs or enhanced TRLs in a pharmaceutical formulation for administration to a subject disclosed herein. In some embodiments, the kit comprises instructions for how to formulate the population of TRLs or enhanced TRLs to produce the pharmaceutical formulation prior to administration to the subject. In some embodiments, the kit further comprises an additional therapeutic agent or active agent disclosed herein for treatment of the disease or the condition. In some embodiments, the disease or the condition comprises cancer. In some embodiments, the kits described herein comprise a pharmaceutical formulation disclosed herein, comprising a population of TRLs disclosed herein. In some embodiments, the TRLs can be further engineered to express a T-cell or chimeric antigen receptor (e.g., enhanced TRLs) as described herein. In some embodiments, the kits further comprise an additional therapeutic agent (e.g., anti-PD1 antibody) such as those disclosed herein. In some embodiments, the kit further comprises instructions for administering the pharmaceutical formulation and/or additional therapeutic agent to the subject to treat a disease or a condition in the subject such as cancer. In some embodiments, the kit comprises instruction that may direct healthcare providers how to treat a subject disclosed herein with the components of the kit in a medical facility or in a point of care capacity. In some embodiments, the kit comprises instructions for administering the composition to a subject in need thereof. In some embodiments, the kit comprises instructions for further engineering the composition to express a biomolecule (e.g., a therapeutic agent). In some embodiments, the kit comprises instructions thawing or otherwise restoring biological activity of the composition, which may have been cryopreserved, lyophilized, or cryo-hibernated during storage or transportation. In some embodiments, the kit comprises instructions for measure viability of the restored compositions, to ensure efficacy for its intended purpose (e.g., therapeutic efficacy if used for treating a subject).

In some embodiments, kits may comprise one or more of the compositions disclosed herein, e.g., the microfluidic device, the culture and/or media, the fluid sample obtained from the subject, or the engineered TCR (e.g., CAR), or any combination thereof. The kits disclosed herein may comprise instructions for separating the TRLs from the fluid sample and/or utilizing the TRLS for a therapeutic application (e.g., treating cancer).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package may be a glass vial or prefilled syringes used to contain suitable quantities of the pharmaceutical composition. The packaging material has an external label which indicates the contents and/or purpose of the kit and its components.

### V. Definitions

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some embodiments, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

Nucleic acids and/or nucleic acid sequences are “homologous” when they are derived, naturally or artificially, from a common ancestral nucleic acid or nucleic acid sequence. Proteins and/or protein sequences are homologous when their encoding DNAs are derived, naturally or artificially, from a common ancestral nucleic acid or nucleic acid sequence. The homologous molecules can be termed homologs. For example, any naturally occurring proteins, as described herein, can be modified by any available mutagenesis method. When expressed, this mutagenized nucleic acid encodes a polypeptide that is homologous to the protein encoded by the original nucleic acid. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The terms “identical” or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol, 48:443 (1970); by the search for similarity method of Pearson and Lipman, Proc. Nat. Acad. Sci U.S.A., 85:2444 (1988); by computerized implementations of these algorithms (including, but not limited to CLUSTAL in the PC/Gene program by Intelligentics, Mountain View Calif, GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CAB OS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment.

In some embodiments, the polypeptides herein are at least 80%, 85%, 90%, 98% 99% or 100% identical to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or 100% identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

A “functional variant” of a protein disclosed herein can, for example, comprise the amino acid sequence of the reference protein with at least or about 1, 2, 3, 4, 5, 6, 7, 8, 9, 10, 11, 12, 13, 14, 15, 16, 17, 18, 19, or 20 conservative amino acid substitutions. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge may be maintained; glutamic acid for aspartic acid and vice versa such that a negative charge may be maintained; serine for threonine such that a free-OH can be maintained; and glutamine for asparagine such that a free ˜NH2 can be maintained.

Alternatively, or additionally, the functional variants can comprise the amino acid sequence of the reference protein with at least one non-conservative amino acid substitution. “Non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution may enhance the biological activity of the functional variant, such that the biological activity of the functional variant is increased as compared to the parent CAR.

Proteins disclosed herein (including functional portions and functional variants thereof) may comprise synthetic amino acids in place of one or more naturally-occurring amino acids. Such synthetic amino acids are known in the art, and include, for example, aminocyclohexane carboxylic acid, norleucine, a-amino n-decanoic acid, homoserine, S-acetyl aminomethyl-cysteine, trans-3- and trans-4-hydroxyproline, 4-aminophenylalanine, 4-nitrophenylalanine, 4-chlorophenylalanine, 4-carboxyphenylalanine, β-phenyl serine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, a-(2-amino-2-norbornane)-carboxylic acid, α,γ-diaminobutyric acid, α,β-diaminopropionic acid, homophenylalanine, and a-tert-butylglycine.

“Polynucleotide” or “oligonucleotide” as used herein refers to a polymeric form of nucleotides of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs.

“Polypeptide” is used interchangeably with the terms “polypeptides,” “peptide(s)” and “protein(s)” and refers to a polymer of amino acid residues.

“Synthetic” as used herein refers to compounds formed or expressed through a chemical process and/or by human agency, as opposed to those of natural origin.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term “tumor-reactive lymphocytes” refer to immune cells that can recognize and target tumor cells. “Circulating tumor-reactive lymphocytes” refer to tumor-reactive lymphocytes found in the patients' fluid samples (e.g., peripheral blood).

As used herein, the term “engineered” and its grammatical equivalents as used herein can refer to one or more alterations of a nucleic acid (e.g., the nucleic acid within an organism's genome) or a polypeptide. The one or more alterations can include modifications, additions, and/or deletions of genes. An engineered cell can refer to a cell with an added, deleted and/or altered gene.

The term “isolate” refers to the removal of an entity (e.g., a cell, protein, nucleic acid) from its natural environment. It is to be understood, however, that nucleic acids, proteins, or cells may be formulated with diluents or adjuvants and still for practical purposes be isolated. For example, nucleic acids typically are mixed with an acceptable carrier or diluent when used for introduction into cells.

The term “purify” refers to when an entity has been increased in purity, wherein “purity” is a relative term, not “absolute purity.” For example, the purity can be at least about 50%, 60%, 70%, 80%, 90%, 95%, 96%, 97%, 98%, 99% or can be 100%. It is to be understood, however, that nucleic acids and proteins may be formulated with diluents or adjuvants and still for practical purposes be purified. For example, nucleic acids typically are mixed with an acceptable carrier or diluent when used for introduction into cells.

The term “enrich” or “enriching” with reference to cells of the present disclosure refers to isolation of a specific cell population from a larger heterogenous cell sample. For example, the systems, methods and kits may include enrichment of CD103+ circulating TRLs (CTRLs) from a heterologous cell sample. Enriching CD103+ CTRLs may refer to increasing the number or percentage of the CD103+ CTRLs as compared to the total number of cells in or volume of the composition, or relative to other cell types, such as by positive selection based on markers expressed by the population or cell, or by negative selection based on a marker not present on the cell population or cell to be depleted. The term should not be interpreted to require complete removal of other cells, cell type, or populations from the composition and does not require that the cells so enriched be present at or even near 100% in the enriched composition.

The term “sorting” refers to a process of identification, selection, and separation of a cell or cell population of interest from other components in a sample, such as other cells not of interest.

As used herein, a statement that a cell or population of cells is “positive” or “+” for a particular marker refers to the detectable presence on or in the cell of a particular marker, typically a surface marker. When referring to a surface marker, the term refers to the presence of surface expression. Surface expression can be detected by device (e.g., flow cytometry, MACS, microfluidic device) used to analyze characteristics of cells or particles by staining with an antibody that specifically binds to the marker and detecting the antibody, wherein the staining is detectable by the device at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, the term “effective amount” refers to the quantity or a concentration of a composition sufficient to bring about an intended result. The effective amount may be a “therapeutically effective amount,” which refers to a quantity or concentration of a composition sufficient to bring about a therapeutic result (e.g., delay the manifestation, arrest the progression, relieve or alleviate at least one symptom of a disorder treated by the methods of the present disclosure). The intended result may be a desired activity of the composition upon administration of the composition to a subject disclosed herein.

As used herein, the term “pharmaceutically acceptable carrier,” “pharmaceutically acceptable excipient,” “physiologically acceptable carrier,” or “physiologically acceptable excipient” refers to a pharmaceutically acceptable material, composition, or vehicle such as a liquid or solid filler, diluent, excipient, solvent, or encapsulating material. A component may be “pharmaceutically acceptable” in the sense of being compatible with the other ingredients of a pharmaceutical formulation. It may also be suitable for use in contact with the tissue or organ of humans and animals without excessive toxicity, irritation, allergic response, immunogenicity, or other problems or complications, commensurate with a reasonable benefit/risk ratio.

As used herein, the term “administration,” “administering” and variations thereof refer to introducing a composition or agent into a subject. Administration can include concurrent and sequential introduction of a composition or agent. The introduction of a composition or agent into a subject is by any suitable route, including orally, pulmonary, intranasally, parenterally (intravenously, intramuscularly, intraperitoneally, or subcutaneously), rectally, intralymphatically, or topically. Administration includes self-administration and administration by another. A suitable route of administration allows the composition or the agent to perform its intended function. For example, if a suitable route is intravenous, the composition is administered by introducing the composition or agent into a vein of the subject. Administration may be carried out by any suitable route. In some embodiments, the administering is intravenous administration. In some embodiments, the administering is pulmonary administration. In some embodiments, the administering is inhalation.

As used herein, the term “pharmaceutical formulation” refers to a mixture of a composition disclosed herein with other chemical components such as diluents or carriers (e.g., pharmaceutically acceptable inactive ingredients) such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or any combination thereof. The pharmaceutical formulation may facilitate administration of the composition to a subject or a cell. Multiple techniques of administering a compound exist in the art including, but not limited to, oral, injection, aerosol, parenteral, and topical administration.

An “expression vector” or a “vector” is any genetic element capable of introducing an exogenous nucleic acid sequence into a cell or organism. An expression vector can be, for e.g., a plasmid, a chromosome, a virus, a transposon, bacteriophages, or a cosmid. An expression vector can behave as an autonomous unit of polynucleotide replication within a cell (i.e., capable of replication under its own control) or being rendered capable of replication by insertion into a host cell chromosome, having attached to it another nucleic acid sequence, so as to bring about the replication and/or expression of the exogenous nucleic acid sequence. Vectors may contain polynucleotide sequences which are necessary to effect ligation or insertion of the vector into a desired host cell and to affect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors may be capable of directly expressing nucleic acid sequence products encoded therein without ligation or integration of the vector into host cell DNA sequences.

Vector also can comprise a “selectable marker gene.” The term “selectable marker gene,” as used herein, refers to a nucleic acid sequence that allows cells expressing the nucleic acid sequence to be specifically selected for or against, in the presence of a corresponding selective agent.

The nucleic acid sequences and vectors disclosed or contemplated herein may be introduced into a cell by “transfection,” “transformation,” or “transduction.” “Transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation, DEAE-dextran; electroporation; cationic liposome-mediated transfection; tungsten particle-facilitated microparticle bombardment, and strontium phosphate DNA co-precipitation. Phage or viral vectors can be introduced into host cells, after growth of infectious particles in suitable packaging cells, many of which are commercially available.

In some embodiments, the vector is an “episomal expression vector” or “episome,” which is able to replicate in a host cell and persists as an extrachromosomal segment of DNA within the host cell in the presence of appropriate selective pressure. Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP.

“Operably linked” as used herein refers to refers to the physical and/or functional linkage of a nucleic acid (e.g., DNA) segment to another nucleic acid (e.g., DNA) segment in such a way as to allow the segments to function in their intended manners. In a non-limiting example, a DNA sequence encoding a gene product is operably linked to a regulatory sequence (e.g., promoters, enhancers and/or silencers) when the regulatory sequence is capable of modulating transcription of the DNA sequence, directly or indirectly. In another non-limiting example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. In yet another non-limiting example, an enhancer or silencer is operably linked to a DNA sequence coding for a gene product when it is ligated to the DNA sequence in such a manner as to increase or decrease, respectively, the transcription of the DNA sequence. Enhancers and silencers may be located upstream, downstream or embedded within the coding regions of the DNA sequence. A DNA for a signal sequence can be operably linked to DNA coding for a polypeptide if the signal sequence is expressed as a preprotein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

The term “sample,” as used herein, can include any material from which nucleic acids, cells and/or proteins can be obtained. Non-limiting examples of sample include whole blood, peripheral blood, plasma, serum, saliva, mucus, urine, semen, lymph, fecal extract, cheek swab, cells or other bodily fluid or tissue, including but not limited to tissue obtained through surgical biopsy. In some embodiments, the sample comprises tissue from a tumor. In some embodiments, the sample is a fluid sample. In some embodiments, the sample comprises tissue from a tumor. In some embodiments, the sample is whole blood, or plasma or serum derived from whole blood. In some embodiments, the sample is a peripheral blood sample, or plasma or serum derived from the peripheral blood. Alternatively, a sample can be obtained through primary patient derived cell lines, or archived patient samples in the form of preserved samples, or fresh frozen samples.

“CD103”, also known as integrin subunit alpha E or ITGAE, refers to a cell surface protein, such as the protein provided in GenBank: AAI13437.1. It belongs to the integrin family of cell adhesion molecules. Integrins are heterodimeric integral membrane proteins composed of an alpha chain and a beta chain.

“CD3”, also known as Clusters of Differentiation 3, refers to a cell surface protein that is expressed on the surface of immune cells. The CD3 complex consists of several subunits, including CD3 gamma, CD3 delta, CD3 epsilon, and CD3 zeta.

“CD4”, also known as Clusters of Differentiation 4, refers to a cell surface protein that is expressed on the surface of immune cells. The CD4 antigen acts as a coreceptor with the T-cell receptor on the T lymphocyte to recognize antigens displayed by an antigen presenting cell in the context of class II MHC molecules. It is expressed not only in T lymphocytes, but also in B cells, macrophages, granulocytes, as well as in various regions of the brain.

“CD8”, also known as Clusters of Differentiation 8, refers to a cell surface protein that is expressed on the surface of immune cells. The CD8 antigen is a cell surface glycoprotein found on most cytotoxic T lymphocytes that mediates efficient cell-cell interactions within the immune system. The CD8 antigen acts as a coreceptor with the T-cell receptor on the T lymphocyte to recognize antigens displayed by an antigen presenting cell in the context of class I MHC molecules. The coreceptor functions as either a homodimer composed of two alpha chains or as a heterodimer composed of one alpha and one beta chain. Both alpha and beta chains share significant homology to immunoglobulin variable light chains. Multiple transcript variants encoding different isoforms have been found for this gene.

“CD39”, also known as ectonucleoside triphosphate diphosphohydrolase 1 (ENTPD1), refers to a cell surface enzyme expressed on various immune cells. The CD39 is a plasma membrane protein that hydrolyzes extracellular ATP and ADP to AMP. Inhibition of this protein's activity may confer anticancer benefits. Several transcript variants encoding different isoforms have been found for this gene.

“SLC6A19”, also known as solute carrier family 6 member 19, is a gene that encodes a system B (0) transmembrane protein that actively transports most neutral amino acids across the apical membrane of epithelial cells.

“SIDT1”, also known as SID1 transmembrane family member 1, is involved in the transport of small interfering RNA (siRNA) across cellular membranes. The SIDT1 belongs to SID1 family of transmembrane dsRNA-gated channels. Family members transport dsRNA into cells and are required for systemic RNA interference.

The section headings used herein are for organizational purposes only and are not to be construed as limiting the subject matter described.

### EXAMPLES

The following examples are included for illustrative purposes only and are not intended to limit the scope of the inventive concepts.

### Example 1: Profiling and Isolation of Circulating Tumor-Reactive Lymphocytes in Blood for Cancer Immunotherapy

The isolation and expansion of tumor-reactive lymphocytes (TRLs) is an essential procedure for adoptive cell therapy. However, existing expansion approaches require the use of tumor tissue as a starting material, which is invasive and not applicable to patients with unresectable tumors or recurrent metastases. This example has shown that the TRLs exist in peripheral blood circulation at a low frequency during tumor progression and can be selectively isolated with a high-performance microfluidic device. Transcriptomic analysis and tetramer-binding experiments on circulating TRLs (CTRLs) revealed that the expression of CD103 almost exclusively defines the tumor reactivity of CD8 T cells in peripheral blood. This example has further demonstrated that expanded CD8+CD103 cTRLs have comparable therapeutic potency to tumor-infiltrating lymphocytes. In addition, CD8+CD103 cTRLs isolated from patient samples resulted in significant enrichment of intratumoral TCR clonotypes and IFN-γ secreting populations during co-culture. This example demonstrates that TRLs can be isolated using a minimally invasive strategy for therapeutic applications.

### Introduction

The autologous transplantation of tumor-infiltrating lymphocytes (TILs) expanded from resected tumors has become a promising therapeutic modality in the clinic. TIL-based adoptive cell therapy has significant advantages over other allogenic and engineered cell therapies because of its inherent heterogeneity that maximizes the tumor-recognizing T cell receptors (TCRs) while minimizing off-tissue effects. The clinical outcome is extremely encouraging so far—long-term complete responses have been observed in subsets of melanoma patients.

Despite the positive outcomes from pioneering clinical trials, the applicability of TIL-mediated ACT has primarily been demonstrated for metastatic melanoma, where resectable metastatic lesions are often large (>3 cm in diameter), providing an optimal source material for TIL isolation⁴. However, for other solid tumors, such large lesions are not readily accessible. Moreover, in some embodiments excisional surgery may not be an option for patients due to a substantial risk or rapid tumor progression. Recent studies have explored the possibility to apply TIL ACT to other solid tumors, such as renal carcinoma, cervical cancer, and breast cancer. But limited functionality of TILs and reduced response rates were observed. Hence, it appears that the existing TIL expansion workflow is less compatible with the therapeutic modality of other types of solid tumors.

Advances in organoid development and deep sequencing have facilitated the generation of TIL-like tumor-reactive lymphocytes (TRLs) via the co-culture of peripheral blood lymphocytes with tumor-derived organoids or peptide pools from tumor-derived neoantigens¹⁰. However, these approaches still require a resection of primary tumor cells harvested using invasive surgical procedures. In addition, the establishment of organoid⁹ and the synthesis of neoantigen-derived peptides¹⁰ takes several weeks to complete. Overall, the requirement of tumor biopsy and lengthy workflow limits the translational value of these approaches as an alternative to TILs.

A subset of peripheral lymphocytes shares clonotypes with TILs and their expansion is highly correlated with response to treatment. Studies also indicate the presence of TRLs in circulation at a very low frequency in melanoma patients, even prior to immunotherapy. However, all existing approaches rely on neoantigen-derived multimers to identify and isolate such a rare population from circulation, which requires the characterization of tumor neoantigens through invasive biopsy. A biomarker that offers clear discrimination between cTRLs and their non-tumor reactive counterpart remains unexplored.

Rarity remains the key challenge for the comprehensive analysis and therapeutic application of cTRLs, as most molecular approaches require at least several thousand cells as the input and millions of cells are required to test therapeutic efficacy. With levels as low as 0.002% in peripheral T cell populations, it is extremely difficult to enrich cTRLs with high purity and recovery for downstream analysis. This example has focused on the development of microfluidic platforms to enable the analysis and enrichment of extremely rare cells, enabling accurate profiling of their phenotype under conditions with limited cell numbers.

A systematic identification of surface markers to isolate cTRLs using a microfluidic platform was performed. ATCR-mediated labeling workflow to convert a level of tumor reactivity into the degree of magnetization for immunomagnetic cell sorting was established. cTRLs for transcriptomic and clonal comparison from animal models with defined epitopes were purified. It was found that the CD8+CD103 signature almost exclusively defines the tumor-reactive population in circulation. This population has a tissue-resident-like (Trm-like) phenotype and has the capability to re-enter blood circulation from primary tumors and accumulate in secondary tumors. It was demonstrated that the cTRLs have strong potential as a therapeutic using multiple adoptive cell transfer models. It was also confirmed that the enrichment based on CD8+CD103 yields higher tumor reactivity in a cohort of patients by comparing the level of interferon gamma (IFN-γ) secretion and clonal similarity.

### High-Performance Cell Isolation to Enable CTRL Profiling

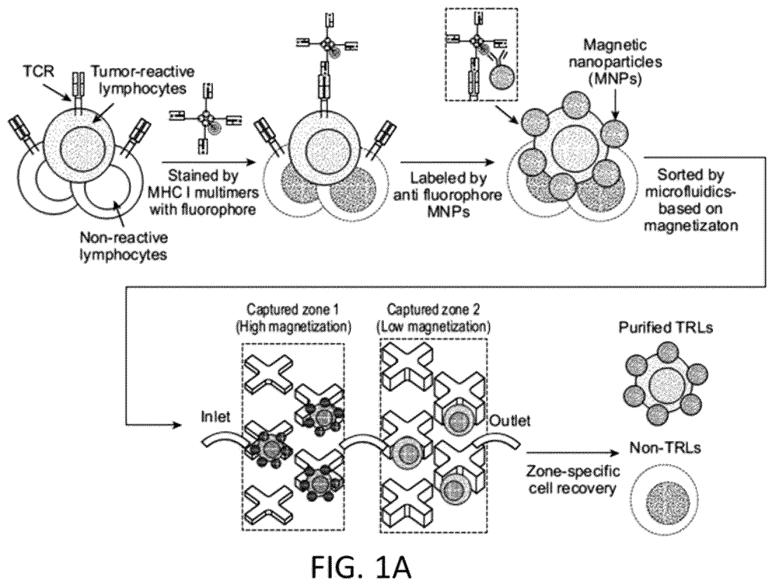

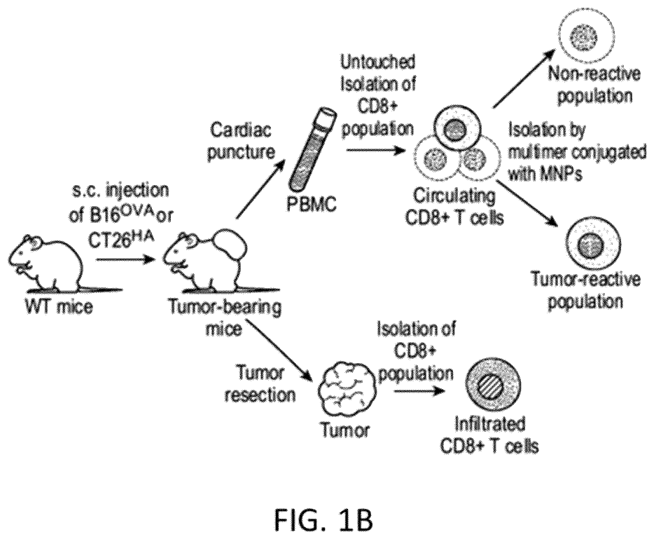

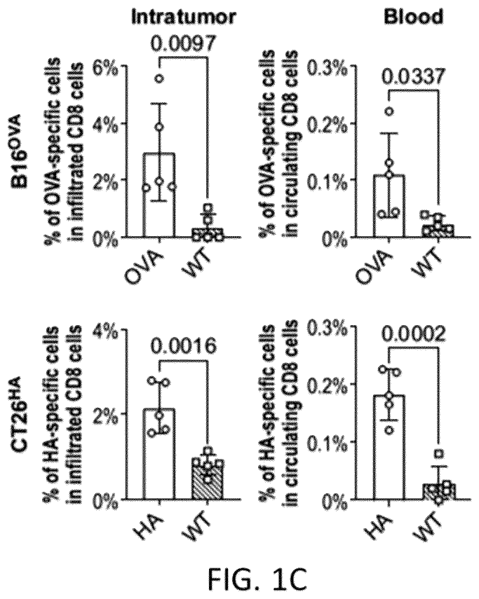

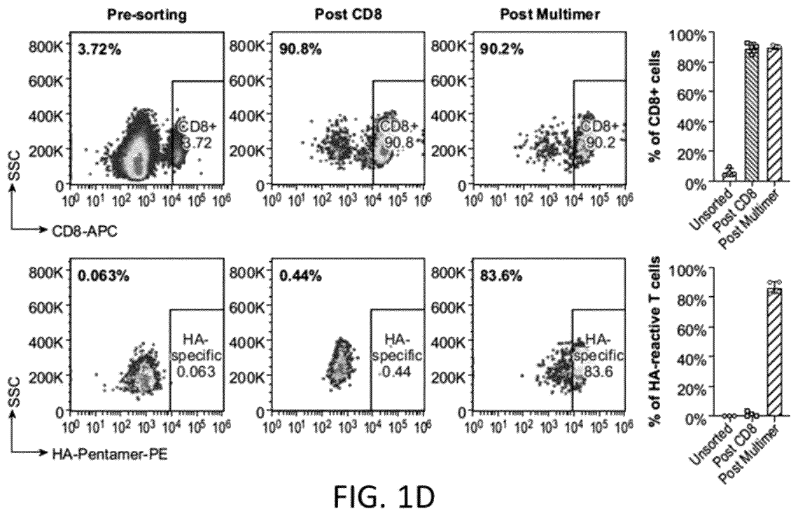

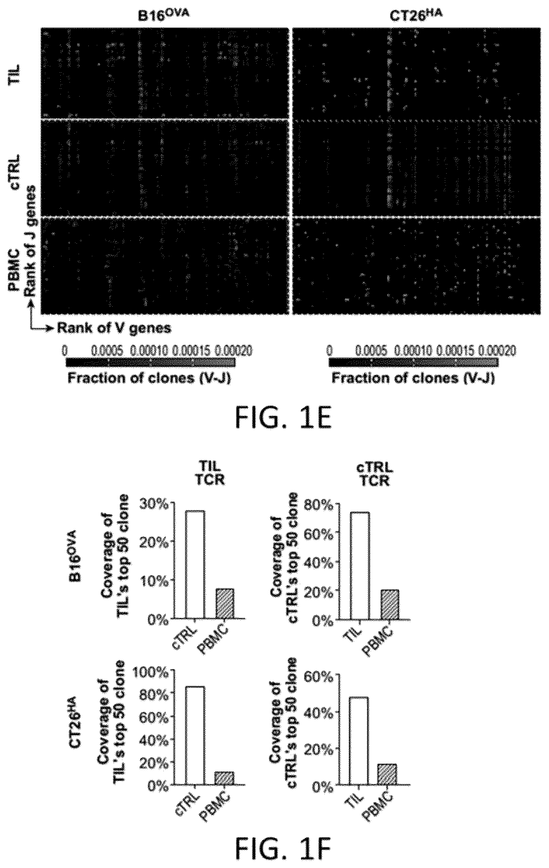

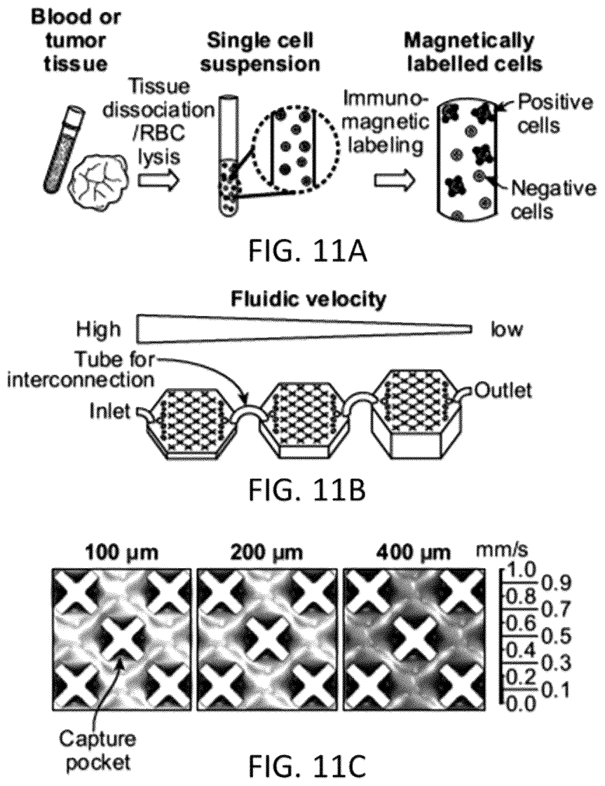

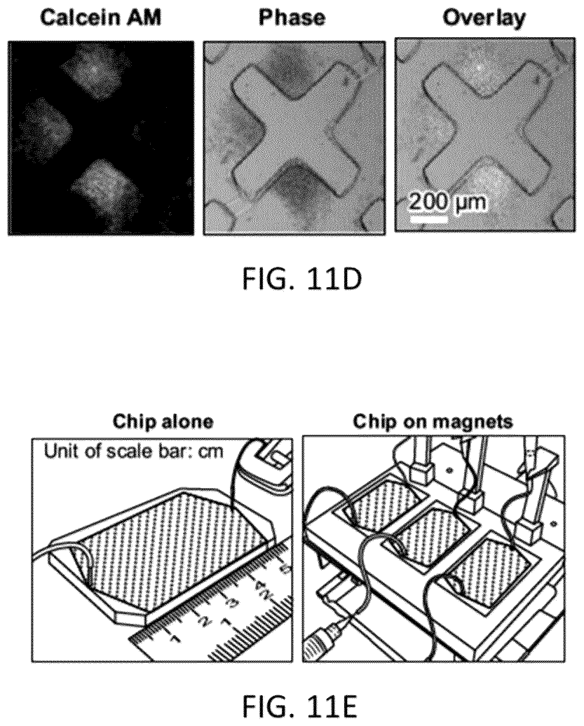

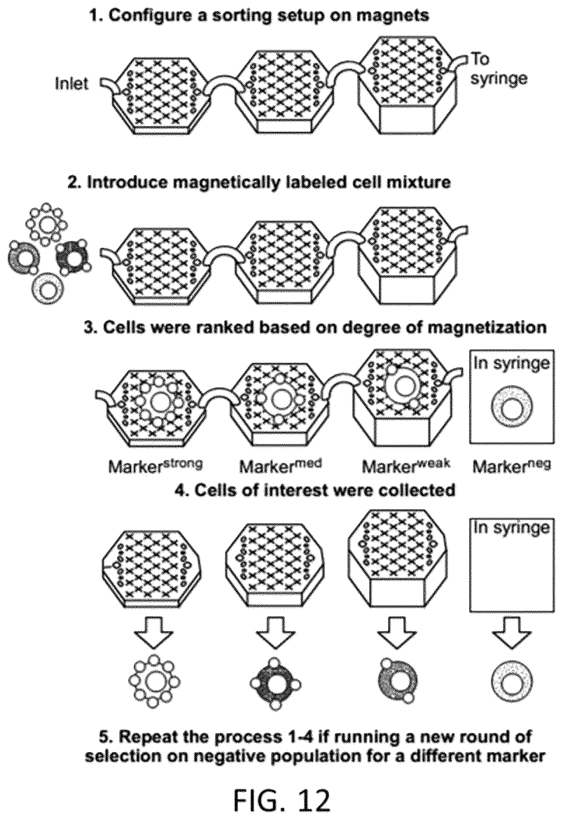

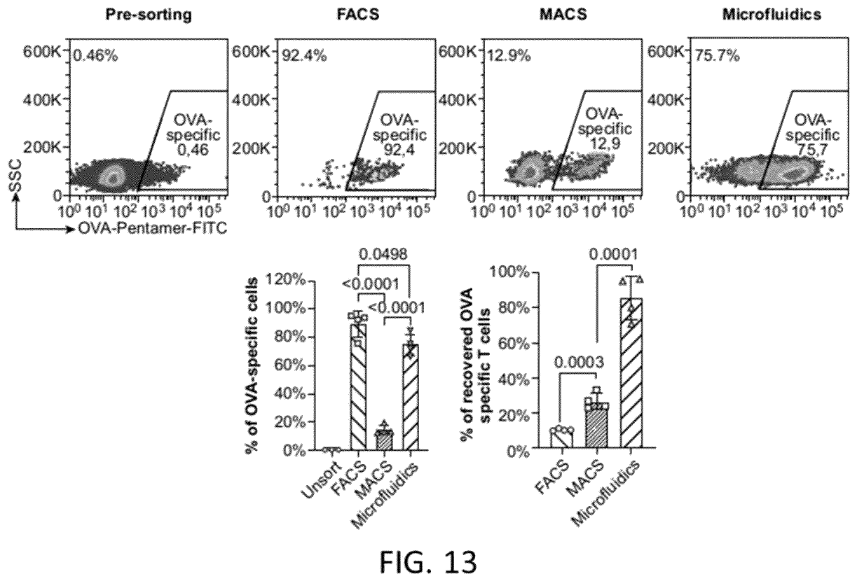

The overall workflow of the tumor reactivity-mediated cell labeling and sorting strategy is illustrated in FIG. 1A. Lymphocytes are treated with major histocompatibility complex (MHC) multimers mimicking a defined tumor epitope to selectively isolate a subset of TRLs with putative tumor reactivity. The multimers are conjugated with a fluorophore, which is used as a linker to attach magnetic nanoparticles (MNPs). To separate the multimer-binding lymphocytes, or tumor-reactive lymphocytes, magnetically labeled cell mixtures are processed with a microfluidic device sandwiched by arrays of magnets (FIGS. 11A-11E). The device contains multiple capture zones that can spatially separate cells with different degrees of magnetization—a higher degree of magnetization results in the capture in a compartment close to the inlet (FIG. 12). In the case of multimer-mediated labeling, the TRLs are captured here while the non-TRL populations are captured in a different compartment. Captured cells can be easily and efficiently recovered from the device compartments when the external magnets are removed. Recovered cells are highly viable and suitable for downstream culture and analysis. As previously reported, the microfluidic cell sorting outperforms commercial cell sorting techniques when recovering rare cell populations. For multimer-mediated labeling and sorting, our microfluidic approach achieved up to 10-fold higher cell recovery while retaining similar purity (FIG. 13).

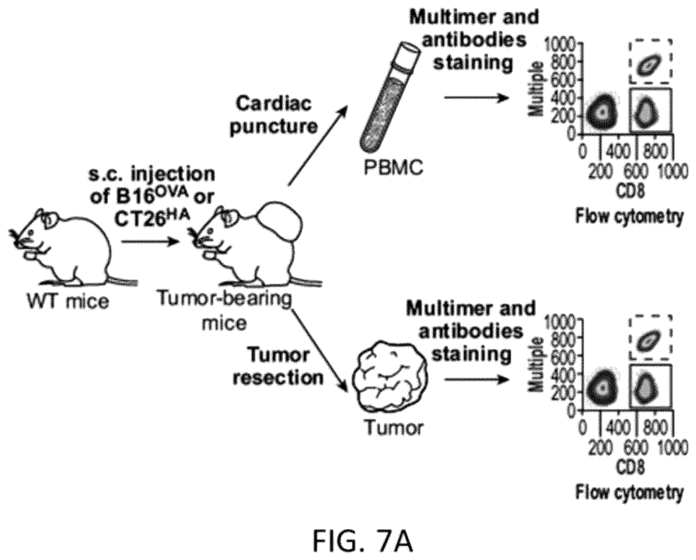

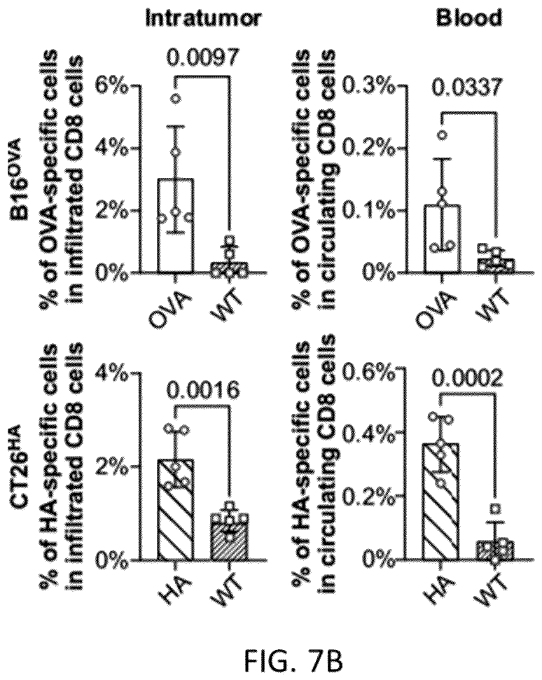

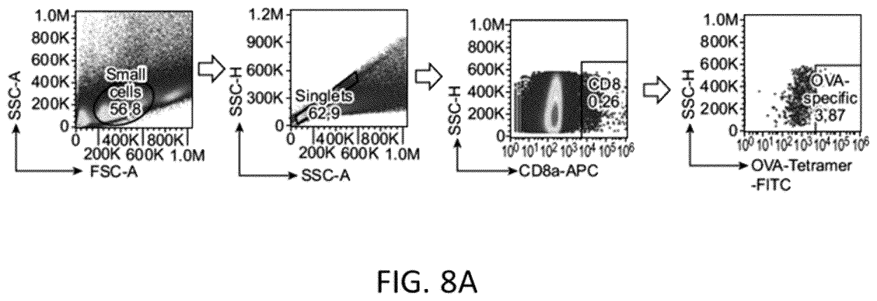

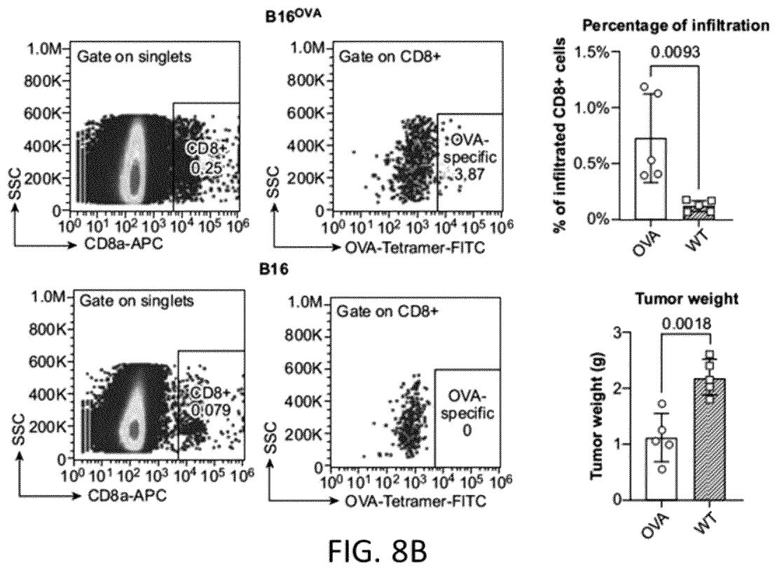

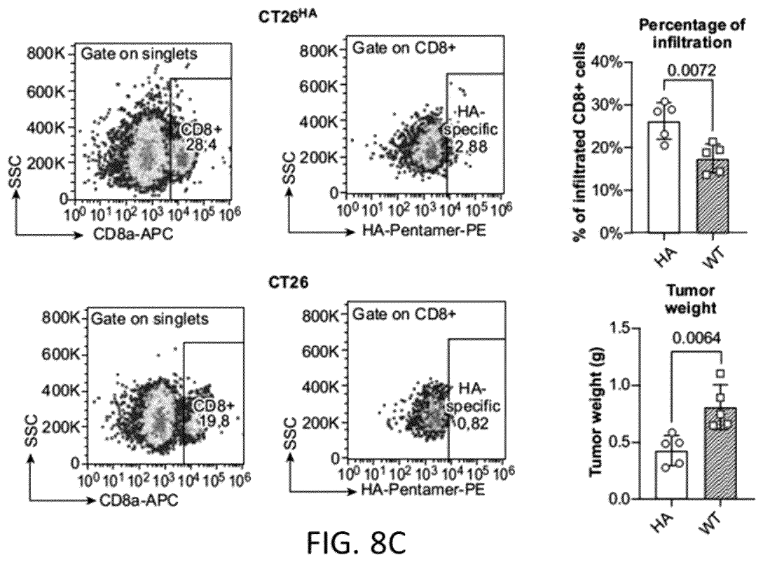

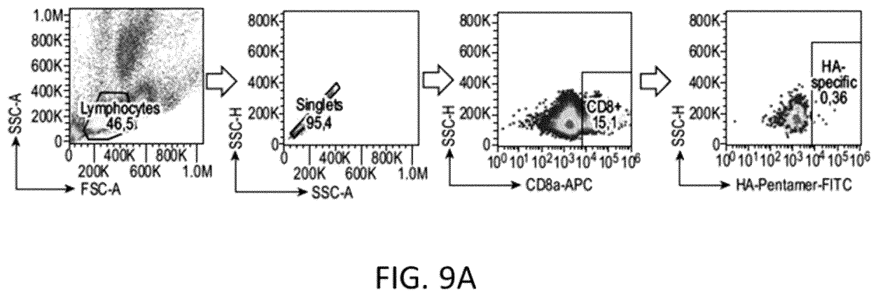

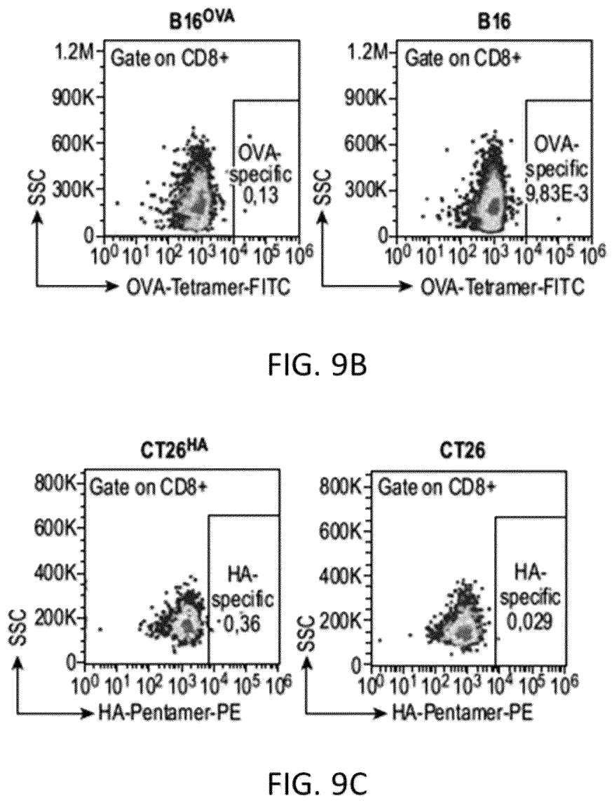

Isolation of cTRLs was pursued using this approach in animal models with two defined highly immunogenic epitopes—chicken ovalbumin (OVA257-264, SIINFEKL) in C57BL6 models and influenza A hemagglutinin (HA533-541, IYSTVASSL) in Balb/c models. Tumor cells with/without the expression of these defined epitopes were injected subcutaneously. Blood and tumor were collected at the mid-late stage (defined by 300-800 mm³ tumor size) of tumor development and CD8+ T cells were labeled by corresponding multimers and antibodies accordingly (FIG. 1B and FIGS. 7A-7B). Flow cytometric analysis indicated that mice bearing tumors expressing immunogenic epitopes exhibited a higher degree of immune response and slower growth of the tumors (FIGS. 8A-8C). The upregulated immune response produced a significantly higher fraction of OVA/HA-reactive T cells presented in tumor and blood (FIG. 1C. FIGS. 9A-9C), which matches a previous observation for melanoma patients. In addition to cytometric analysis, we utilized corresponding multimers with MNPs and microfluidics to purify tumor-reactive T cells. The purity of isolation is 76 and 84% for OVA and HA multimers (FIG. 1D), respectively.

The high yield and purity from microfluidic sorting enabled us to perform direct TCR sequencing on the rare cTRL populations (FIG. 1E). We identified over 1,000 clonotypes (defined by the unique CDR sequence) in the cTRLs. Mapping of V-J usage reveals a striking similarity between cTRLs and intratumoral TILs, rather than the non-reactive portion of PBMCs. In terms of the TCR repertoire, cTRLs cover 30%-85% of the top 50 clones presented in TILs, 3-8 times higher than the coverage observed in PBMC (˜10%, FIG. 1F). Taken together, these data suggest that the cTRLs exist in the circulation during disease progression and share a higher degree of clonal similarity with intratumoral TILs. This indicates the cTRLs may originate or be derived from the same progenitors as intratumoral TILs.

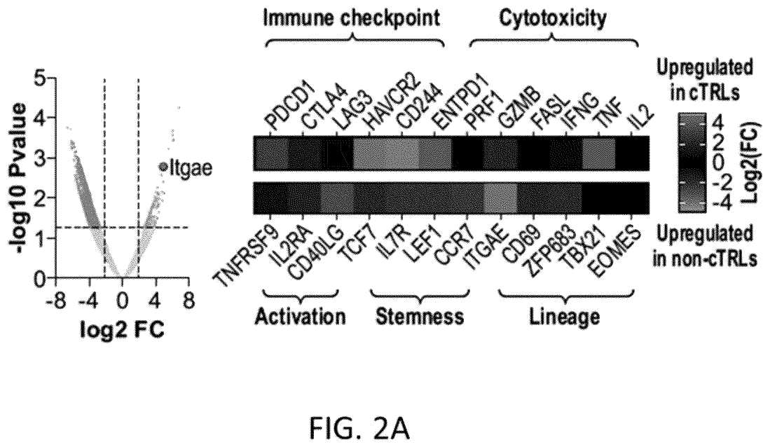

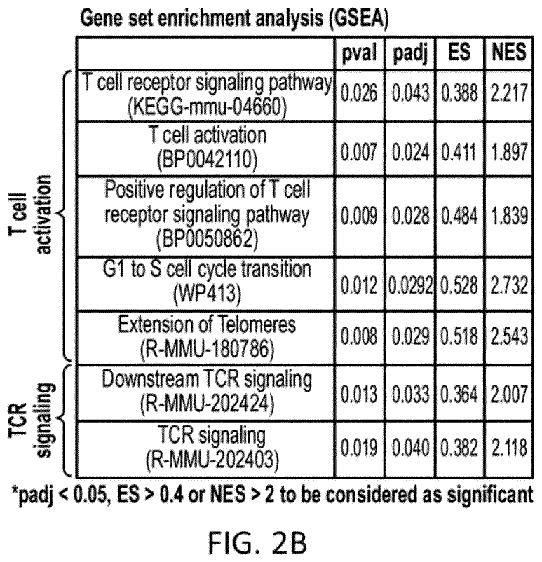

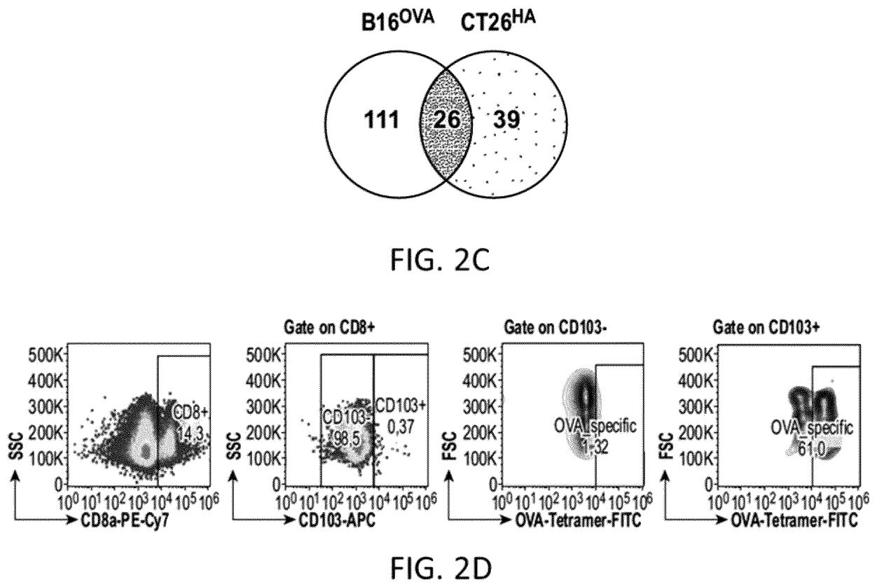

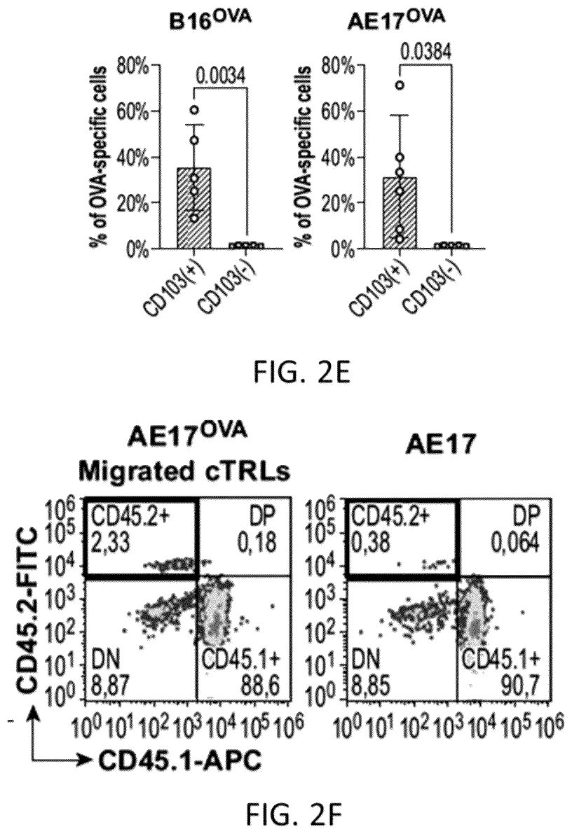

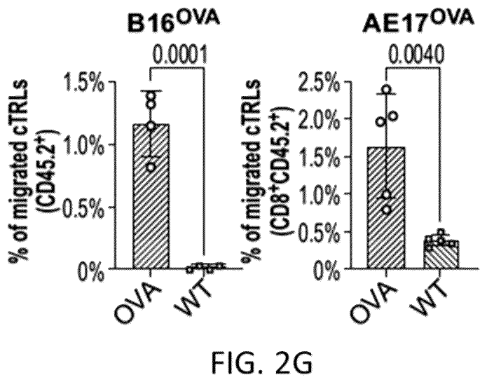

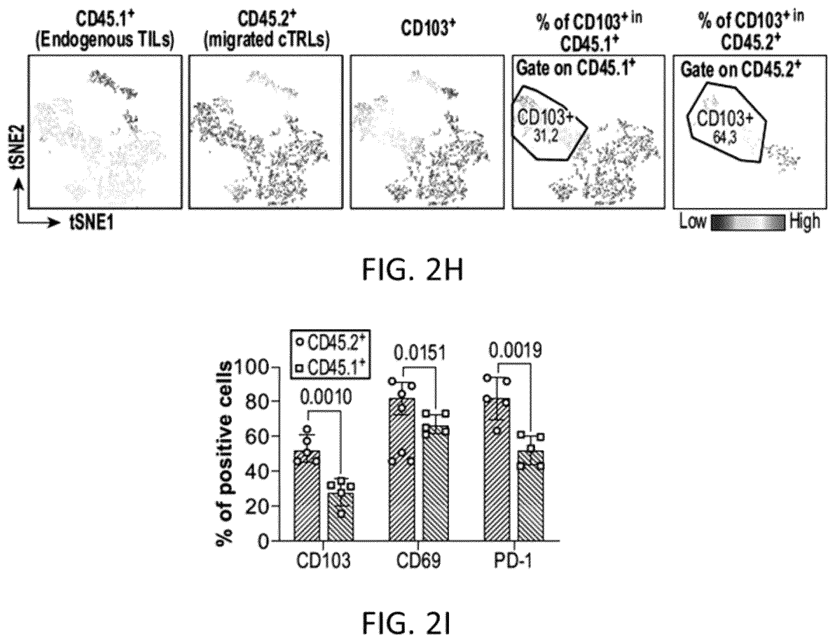

The high yield and purity from microfluidic sorting also allowed RNAseq to be performed on the rare CTRL population. For the OVA-reactive cTRLs isolated from the melanoma model, a memory phenotype was detected with upregulated expression of TCF7, IL7R, and LEF1 (FIG. 2A). In addition, cTRLs had a high expression of ITGAE (CD103) and medium-high expression of CD69. As for lineage-specific transcriptional factors, cTRLs upregulated the expression of ZFP683 (also known as Hobit) but not TBX21 (also known as T-bet) nor EOMES (FIG. 2A, right).

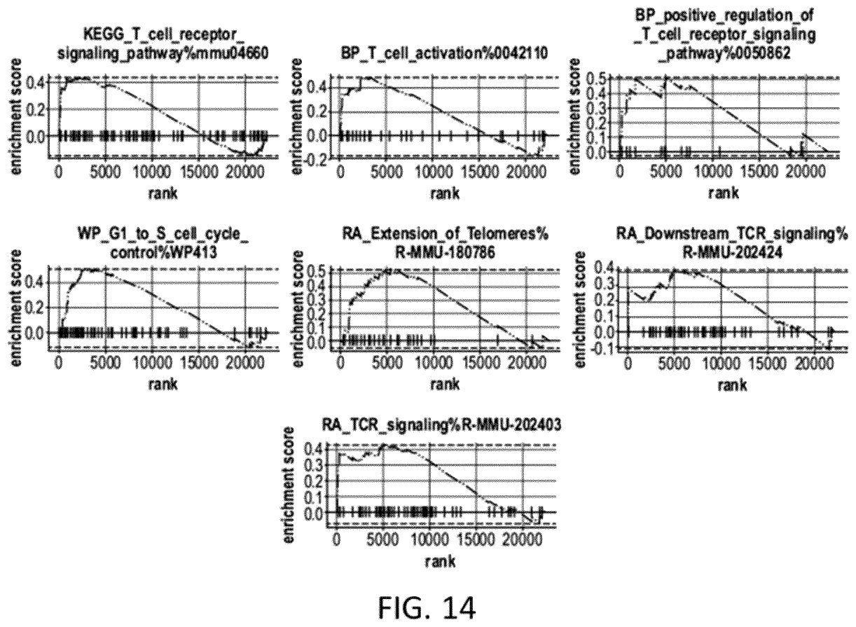

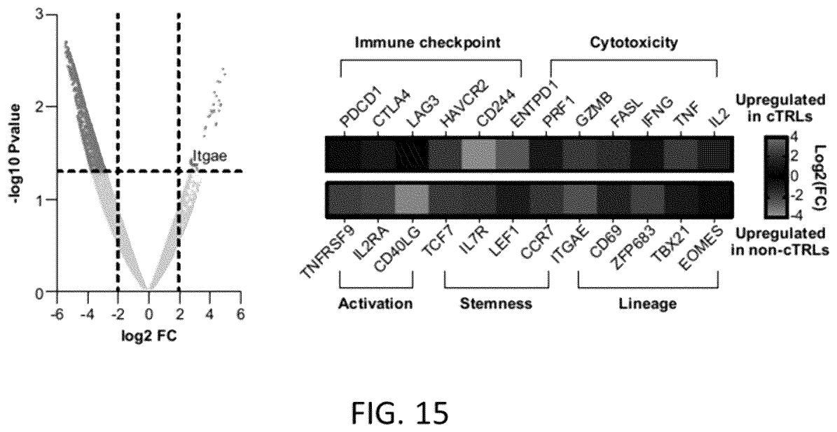

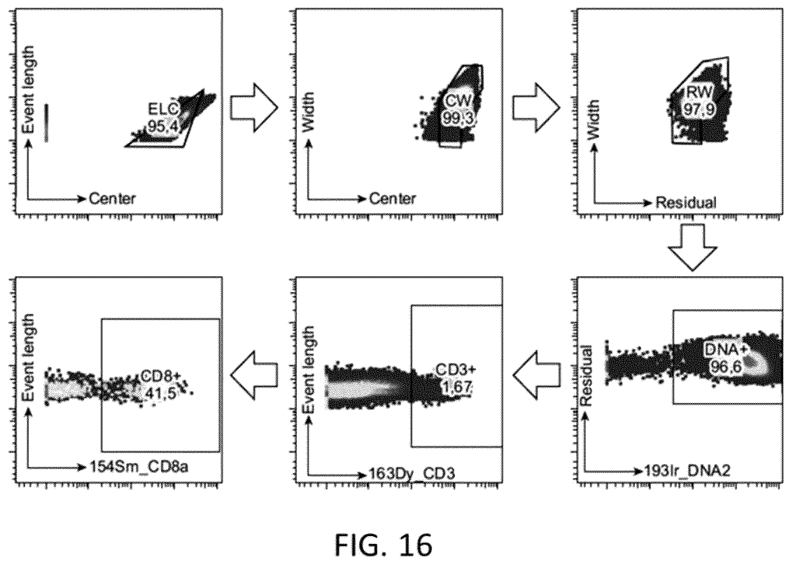

Hence, the cTRLs have a phenotype similar to tissue-resident memory T (Trm) cells. Trm cells are considered the frontline to protect the host at the site of pathogenesis²⁶. Gene set enrichment analysis (GSEA) further suggests that the cTRLs have statistically significant upregulation of T cell activation and TCR signaling pathways (FIG. 2B, FIG. 14). A similar trend of marker expression in HA-reactive cTRLs isolated from the colon cancer model was observed (FIG. 15). By overlaying the differentially expressed genes, 26 shared upregulated genes were identified (FIG. 2C). Genes that are known to be transiently or permanently upregulated during TCR-MHC interaction (e.g., CRTAM27, GGT128, and CD8A29) were eliminated from this subset. One of the strongest hits observed was ITGAE, a gene that encodes CD103. This factor is associated with a migratory phenotype and exhibits low expression across PBMCs³⁰, indicating that it may represent a specific, biologically relevant marker for cTRLs.

To further evaluate CD103 as a marker for CTRL isolation, circulating CD8+ lymphocytes were isolated from mice bearing B16F10 and AE17 cancer cell lines expressing the OVA epitope. Flow cytometric analysis indicated that the OVA-reactive cTRLs are almost exclusively CD103 (FIG. 2D). Collectively, about 30% of the CD8+CD103 cells in circulation are OVA-reactive (FIG. 2E), which yields up to 50-fold enrichment of OVA-reactive T cells. Accordingly, CD103 helps to define tumor-reactive populations in bulk T cells, alone^{31,32} or together with other markers³³, defines T cells with elevated potency for adoptive cell therapy and immune checkpoint blockade. However, it is interesting that such Trm-like phenotype, which is believed to reside in non-lymphoid tissue, is present in circulation.

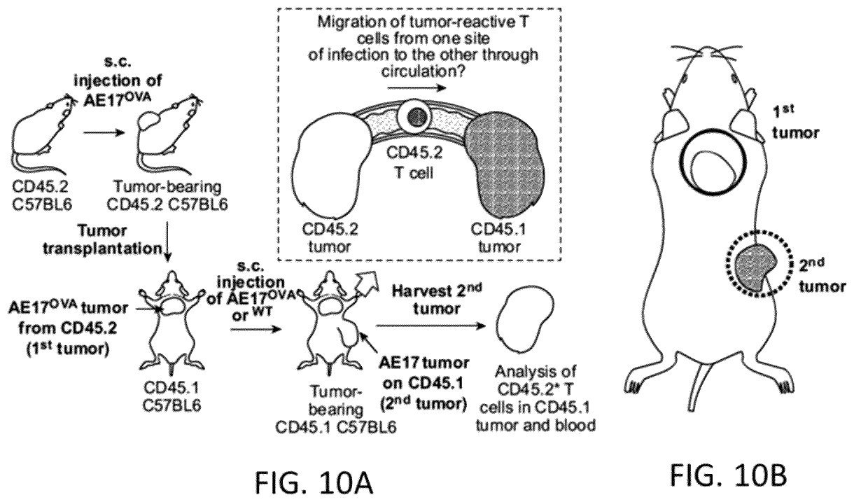

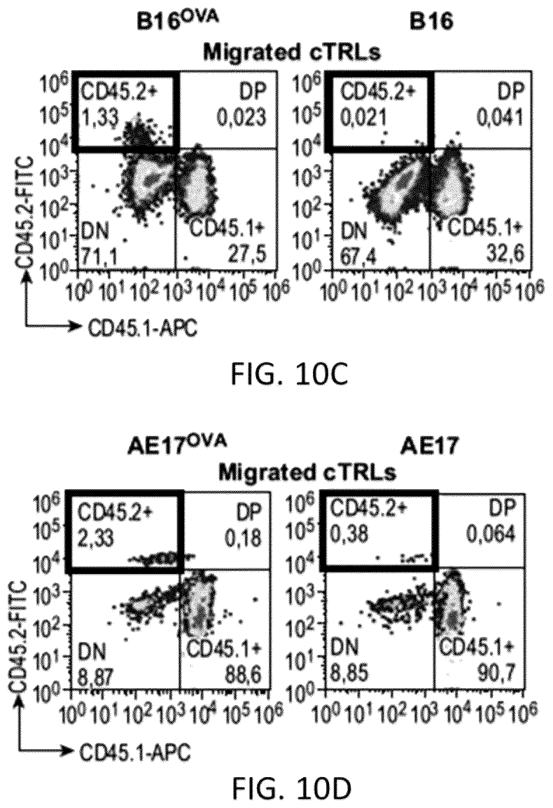

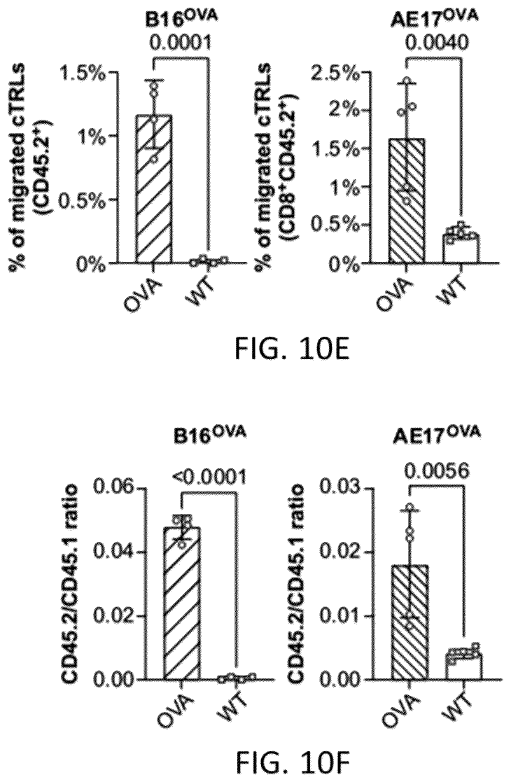

To probe the mechanism underlying the presence of CD8+CD103 CTRLs in circulation, a tumor transplantation model was used to visualize the trafficking of these cells where an OVA-expressing tumor from the donor CD45.2 mice was transplanted as a whole to the host CD45.1 mice bearing a secondary (2nd) tumor with/without OVA epitopes. Two tumors were allowed to grow simultaneously, and the 2nd tumors were collected at the end for flow cytometric analysis (FIG. 2F, FIGS. 10A-10B). For all of the specimens analyzed, we observed a small portion of CD45.2+ cells presented in the 2nd tumor. Since the only source of CD45.2+ T cells is the TILs within the transplanted tumor, this observation provides direct evidence that the TILs can enter the circulation and migrate to distant organs. In addition, the 2nd tumors with OVA epitopes attracted at least a 5-fold higher percentage of CD45.2+ T cells compared to OVA-free WT tumors (FIGS. 2F-2G, FIGS. 10C-10F)—this suggests the migration and the accumulation of cTRLs are antigen-driven. Sites with similar patterns of infection (e.g., same immunogenic epitope) attract more cTRLs to re-infiltrate and reside. The molecular signature of migrated cTRLs in the 2nd tumor by CyTOF was also examined. Compared to the CD45.1+ endogenous T cells, CD45.2+ CTRLs had higher expression of CD103, CD69+, and PD-1+ (FIGS. 2H-2I), suggesting they retained the Trm-like, activated phenotypes after migration.

Taken together, the data collected indicates that the Trm-like cTRLs in blood migrate towards and accumulate specifically in distal tumors. This observation is consistent with the emerging evidence showing Trm enter circulation to increase the overall immune response. Mechanistically, CD103 is an integrin protein that binds to E-cadherin and governs the formation of cell protrusions/filopodia, an essential component for initiating cell migration. This points to the critical role of CD103 in cell motility and moreover, CD103 TRLs are reported to have elevated energetic potential and greater migration capacity. Hence, the phenotypic properties of CTRLs are consistent with prior observations of CD103 T cells.

Expansion and Administration of cTRLs to Test Therapeutic Activity

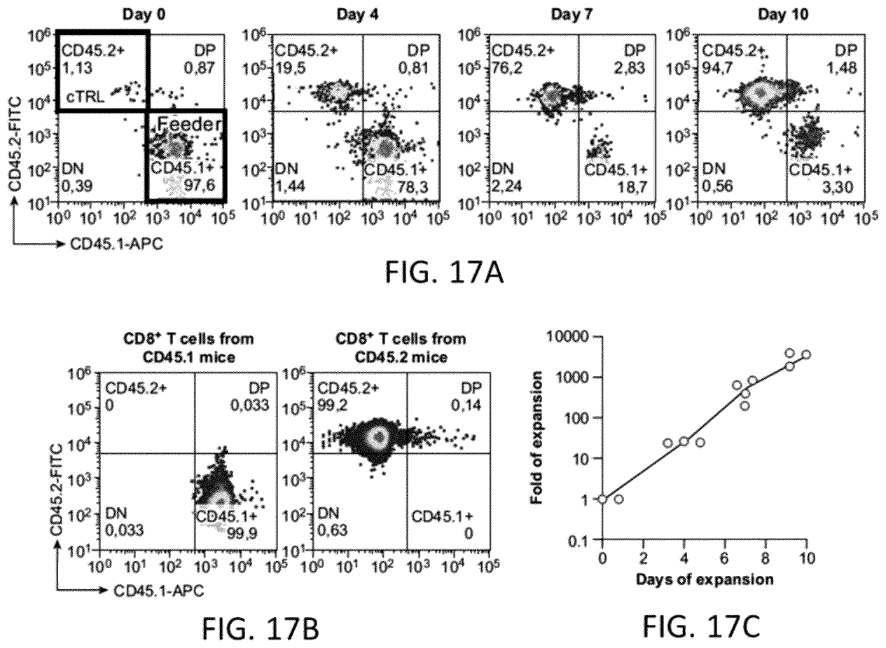

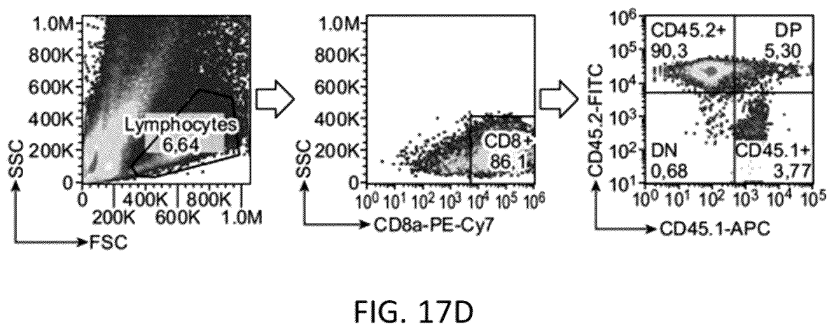

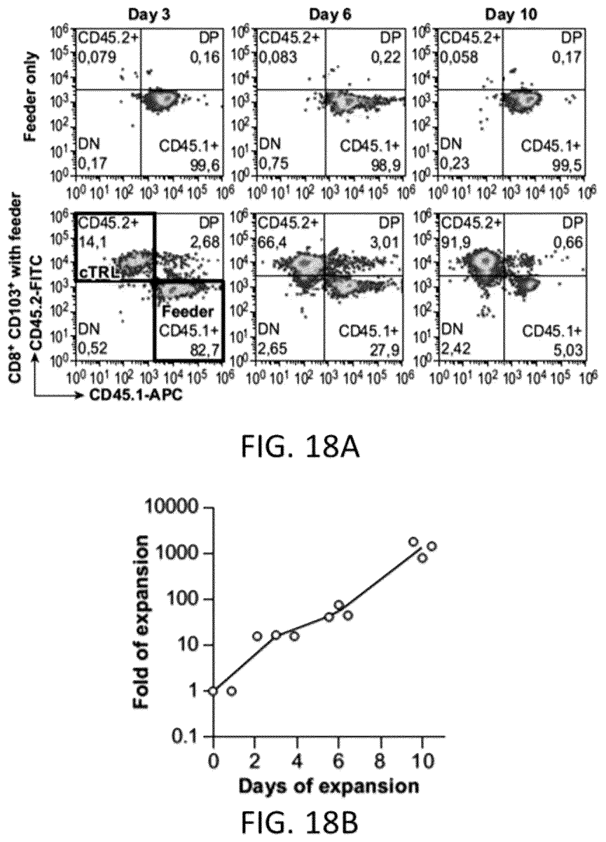

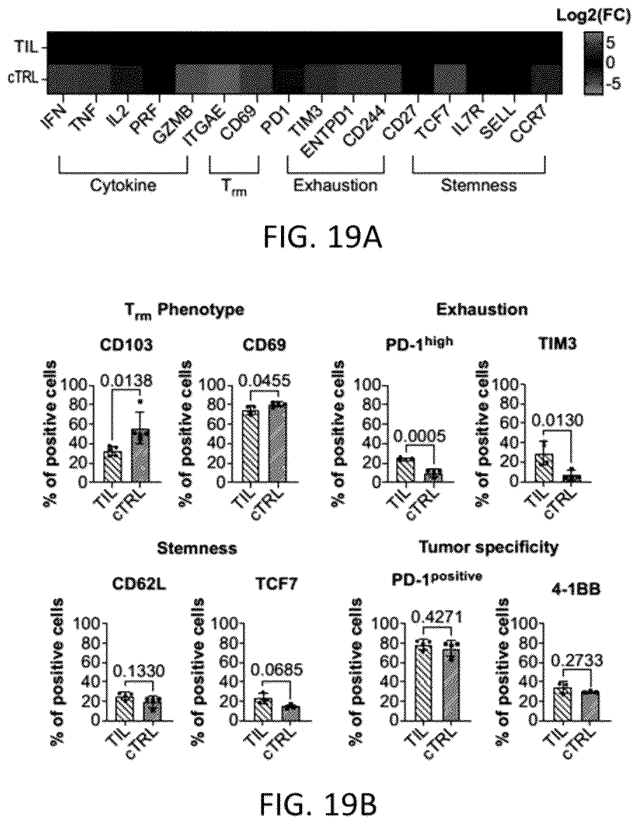

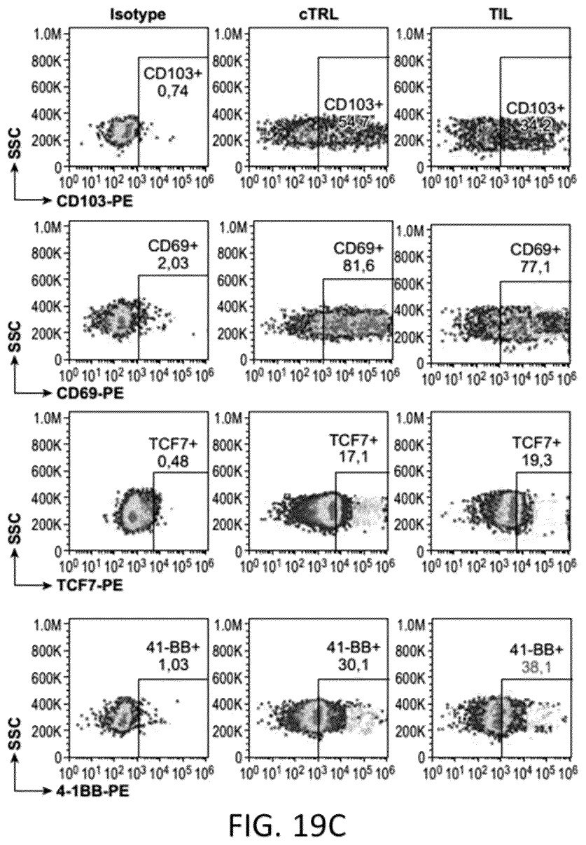

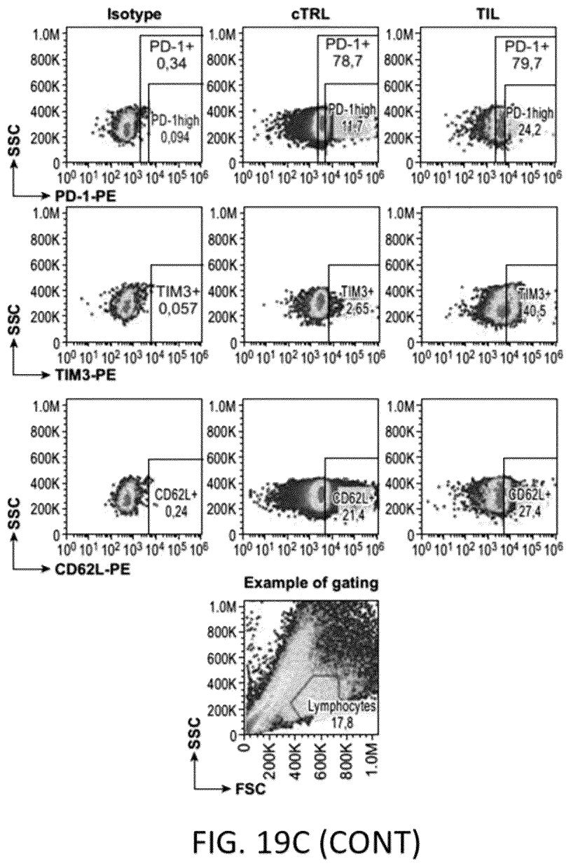

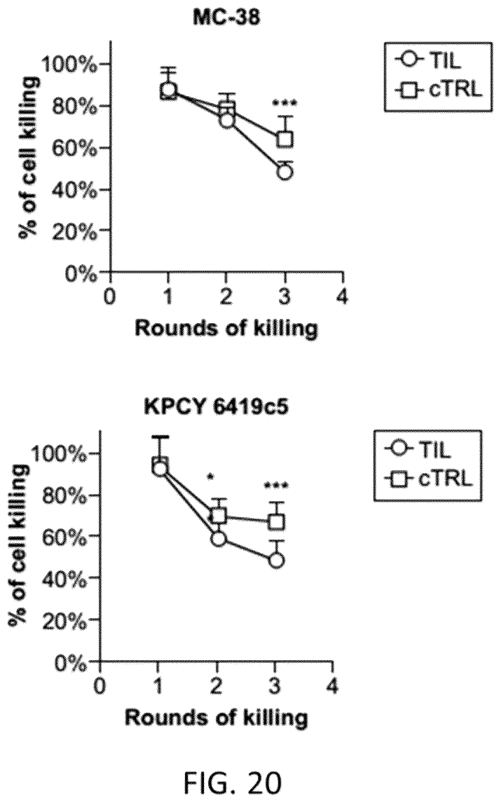

The ‘homing’ behavior of cTRLs toward a specific tumor microenvironment makes them an attractive candidate for adoptive cell therapy. Therefore, the rapid expansion protocol (REP) of rare tumor-reactive TILs was adapted and achieved up to 2000-fold expansion of cTRLs in 10 days, yielding the final number of cTRLs around 0.1-4 million per mouse (FIGS. 17A-17D and FIGS. 18A-18B). The cTRLs maintained their Tᵣₘ-like phenotypes upon expansion, including a high expression of critical surface (CD103 and CD69) and intracellular cytotoxic markers (IFNG and GZMB) (FIGS. 19A-19C). It is also worth noting that about 70% of the cTRLs are PD-1medTIM3-, indicating a partially exhausted yet tumor-reactive phenotype40,41. An in vitro killing assay revealed that expanded cTRLs lysed 65% of the cells in 24 hrs. Such killing potency is comparable to TILs. We also elaborated a continuous antigen exposure model⁴² to assess the degree of exhaustion (FIG. 20). Compared to TILs subjected to REP, expanded cTRLs showed greater killing potency over 3 rounds of antigen exposure, suggesting a strong anti-exhaustion phenotype. This result is in line with a recent pan-cancer analysis suggesting that the CD8+ ZNF683+ Trm subpopulations have a lower frequency of terminally exhausted T cells.

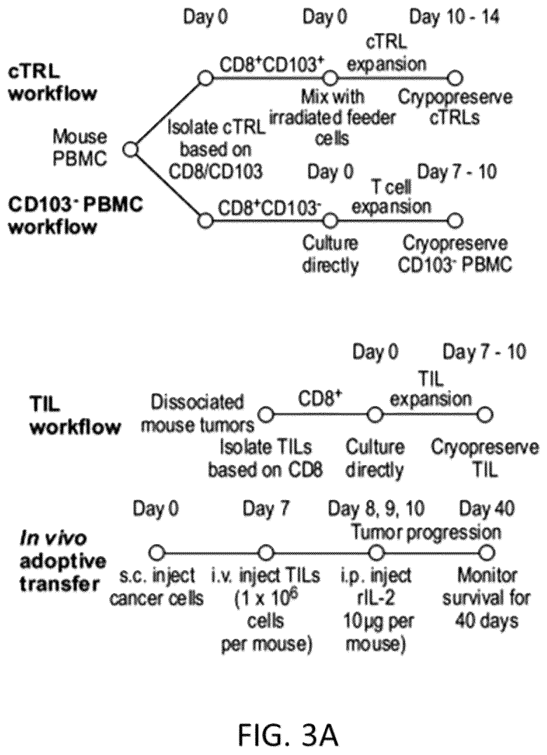

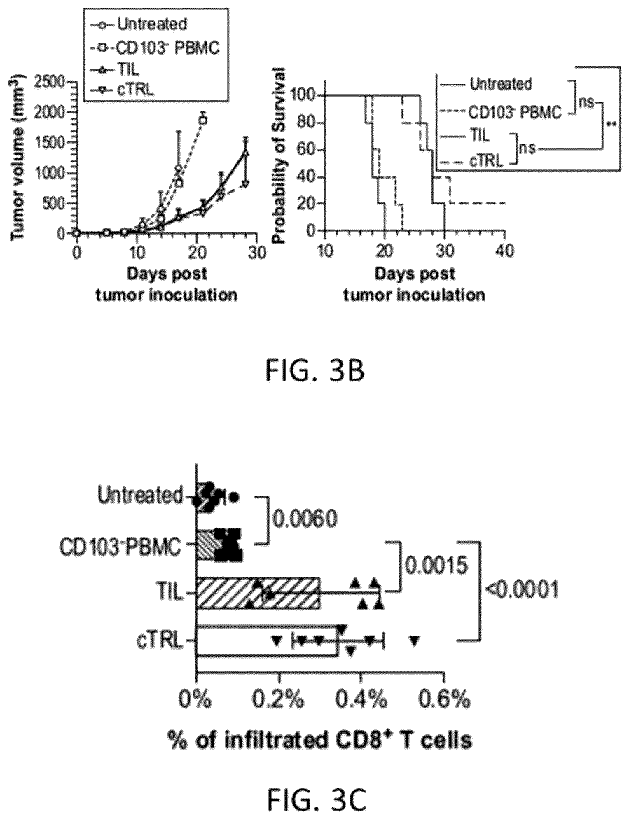

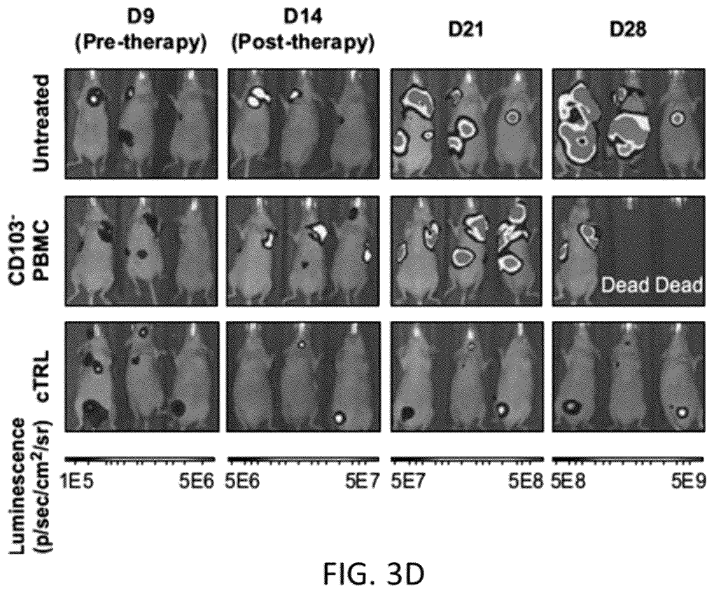

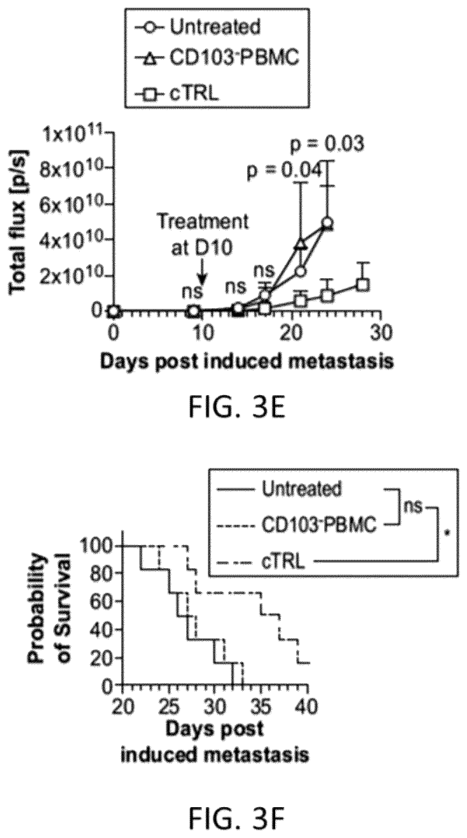

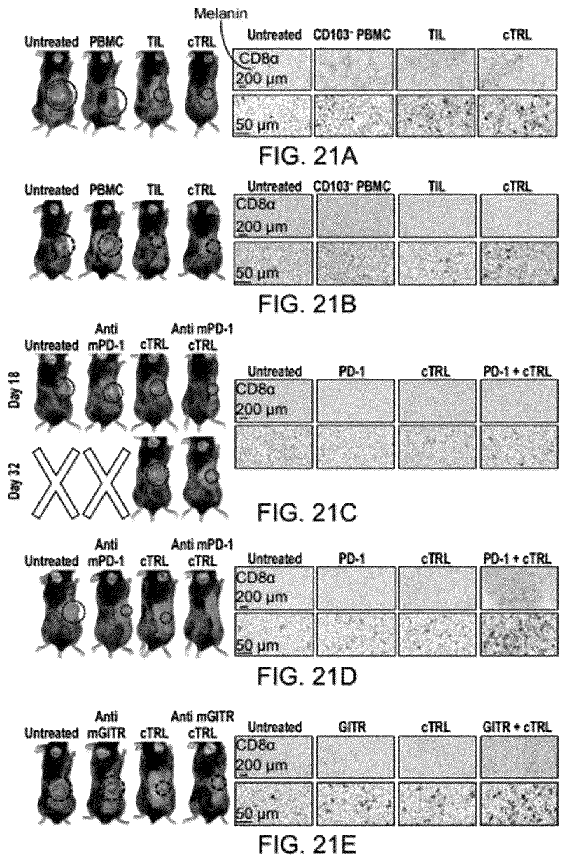

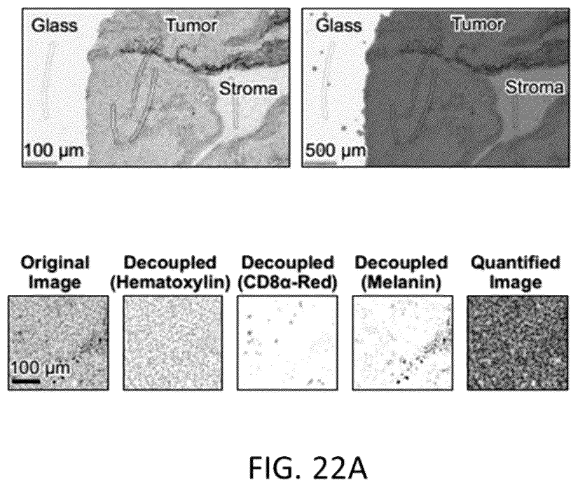

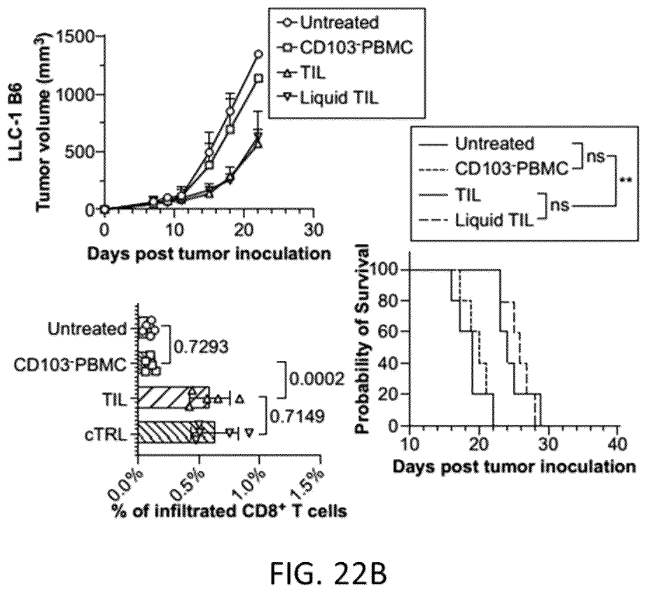

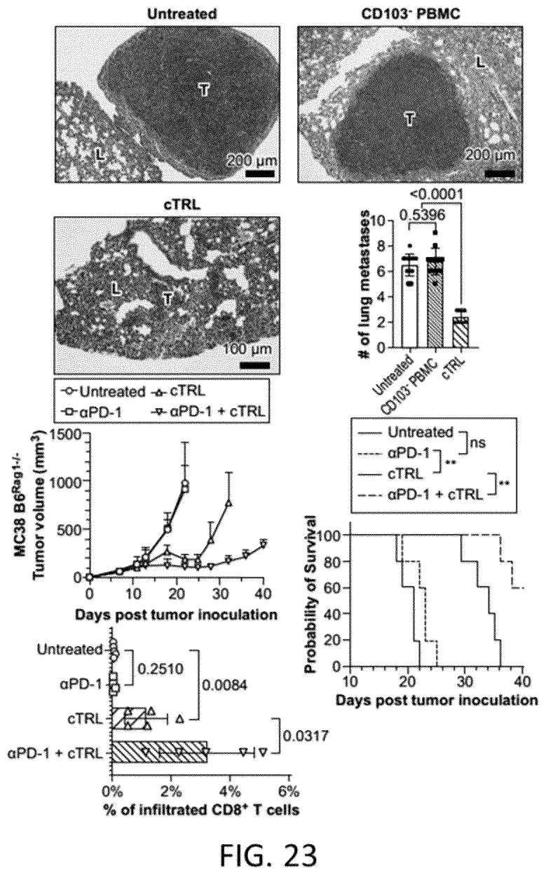

We next characterized the therapeutic efficacy of cTRLs in vivo using multiple animal models. We first benchmarked the therapeutic potency of expanded TILs, CTRLs, and CD8+CD103− PBMC populations with subcutaneous B16F10 melanoma (FIGS. 3A-3C, FIGS. 21A-E, and FIG. 22A) and LLC1 NSCLC model (FIG. 22B). We confirmed that cTRLs have improved therapeutic potency compared to CD8+CD103− PBMCs. It is worth noting that the potency of cTRLs is comparable to the TILs isolated from solid tumors-both extending median survival by 40%-50%. This suggests that the TCR repertoire of the cTRLs, although not as diverse as the TILs, exhibits sufficient tumor-reactive TCRs for tumor killing. In addition, we verified the feasibility of treating metastases with cTRLs using an induced metastasis model by tail-vein injection of 4T1 cells (FIGS. 3D-3F)—the group treated by cTRLs had a 30% increase in the median survival and a 60% reduction in the lung metastases (FIG. 23, top) compared to the CD8+CD103− PBMC group. It is worth noting that no primary tumor was involved during the CTRL isolation and reintroduction—this suggests that the use of cTRLs is applicable to a broader group of patients, including individuals with unresectable tumors or who underwent surgery (without TIL isolation/expansion) and went to developed recurrent metastasis. Taken together, our findings indicate that the cTRLs have significant therapeutic potency and could be used in conjunction with a more practical harvesting process. However, we observed that monotherapy using cTRLs was insufficient for long-term tumor management, with less than 20% of the mice in this study experienced a complete response (CR) at study endpoint.

To further improve the therapeutic efficacy of cTRLs, the combination of cTRLs with immune checkpoint blockade (ICB) was tested. It was hypothesized that the cTRLs would respond to the ICB considering their PD-1medTIM3-partially exhausted phenotype. In addition, existing studies showed that the percentage of CD8′CD103′ Trm predicts response to ICB therapy-further supporting this rationale^{31,32}. To test this hypothesis, we elaborated a MC38 colon cancer model in immunocompromised mice (RAG−/−) and treated them with the combination of cTRLs and anti-PD-1 blockade (FIG. 23, bottom 3 graphs), to solely test the effects of ICB on infused CTRLs. Not surprisingly, we observed no difference between the untreated and anti-PD-1 groups in immunocompromised backgrounds, as anti-PD-1 blockade requires the T cells to function. Monotherapy of cTRLs produced transient tumor control for about 2 weeks but underwent rapid tumor progression after this time point. A cocktail of anti-PD-1 and cTRLs was observed to exhibit durable tumor control over 4 weeks and extended the median survival by 100% compared to the untreated group. IHC analysis comparing the resected tumors from the cTRLs, and the cocktail revealed a large difference in the number of infiltrated CD8′ T cells (1.2% vs 3.2%). Taken together, this immunocompromised model provides direct evidence that the cTRLs are responsive to ICB and the cocktail of cTRLs and ICB can achieve long-lasting tumor control.

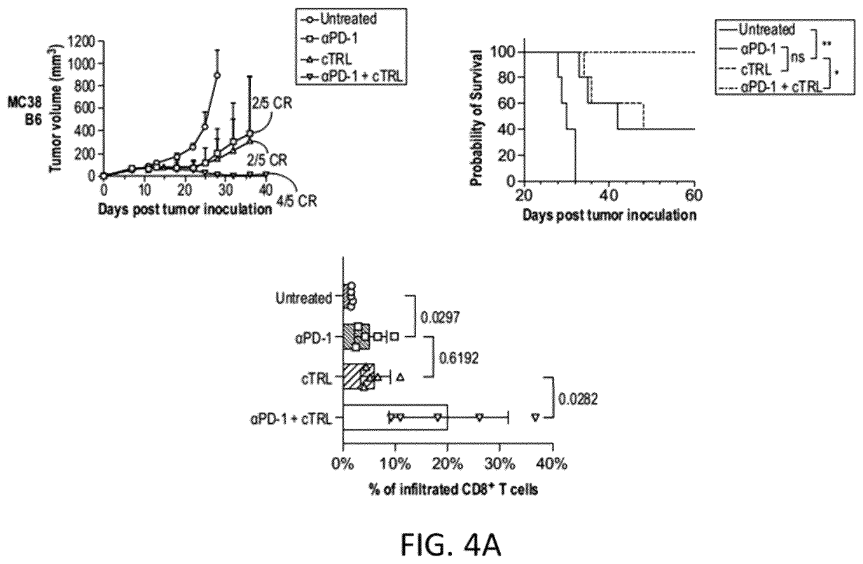

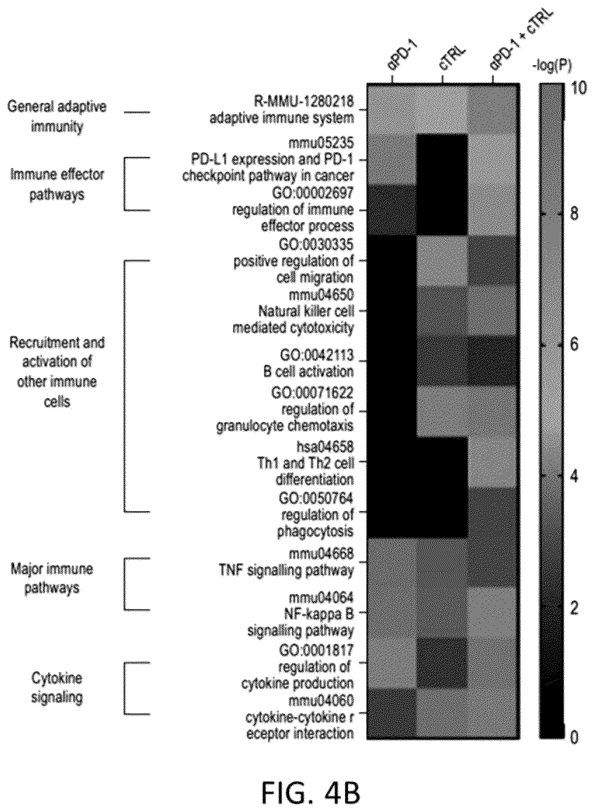

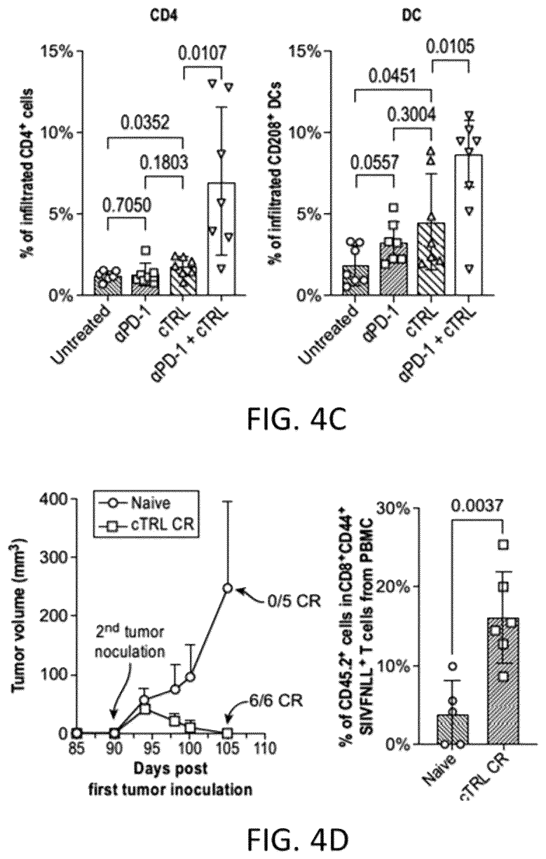

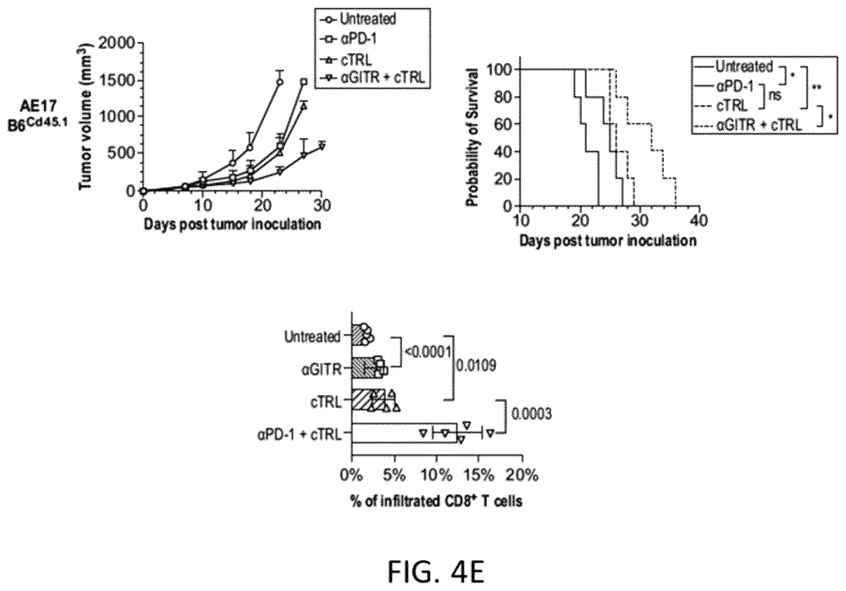

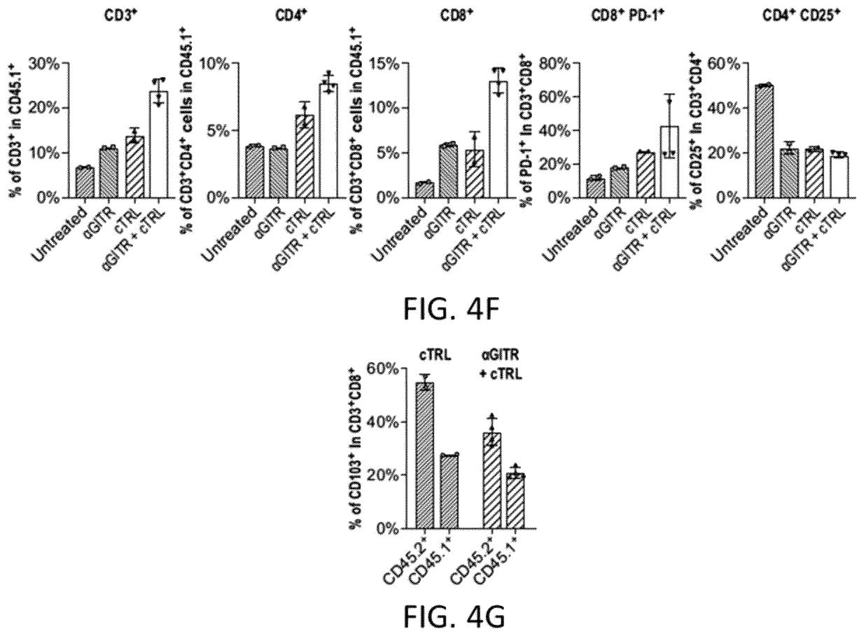

In addition to direct tumor killing, Trm is also known to utilize other mechanisms for tumor control, such as the new recruitment of other immune cells. To gain insights related to these indirect processes, we used a MC38 model in immunocompetent mice (CD45.1+) and treated them with the CD45.2+ CTRLs and ICB (FIG. 4A). With intact endogenous immunity, the cocktail of cTRLs and ICB achieved remarkable tumor control, with 5 out of 5 mice surviving at study endpoint and 4 mice exhibiting CR. To better understand how cTRLs and ICB altered the tumor microenvironment (TME), pathway enrichment was performed on the upregulated genes from gene expression analysis (FIG. 4B, FIG. 14). Although both treatments upregulated the overall adaptive immune response, the type of enriched immune pathways was different between the treatments, with PD-1 ICB significantly altering the PD-1/L1 pathways and enhancing the immune effector functions, while cTRLs promoted migration and activation of other immune cells, including granulocytes, B cells, and NK cells. The cocktail of PD-1 ICB and cTRLs further boosted the differentiation of helper T cells and phagocytosis. IHC analysis on CD4 and CD208 (DC-LAMP) further confirmed this observation at the protein level (FIG. 4C). Taken together, these data indicate that cTRLs prompt additional immune cells to infiltrate while ICB can improve the functionality of infiltrated immune cells—hence co-administration of cTRLs and ICB shows synergistic effects. In addition, it is worth noting that the antitumor immunity against the MC-38 tumor is durable as observed through tumor rechallenge experiments conducted 3 months post initial therapy (FIG. 4D). Flow cytometric analysis using a MC-38 derived multimer (SIIVFNLL) confirmed the presence of tumor-reactive memory T cells of both CD45.1+ and CD45.2+ origin, providing a direct evidence that the cTRLs participate in the establishment of long-term anti-tumor immunity.

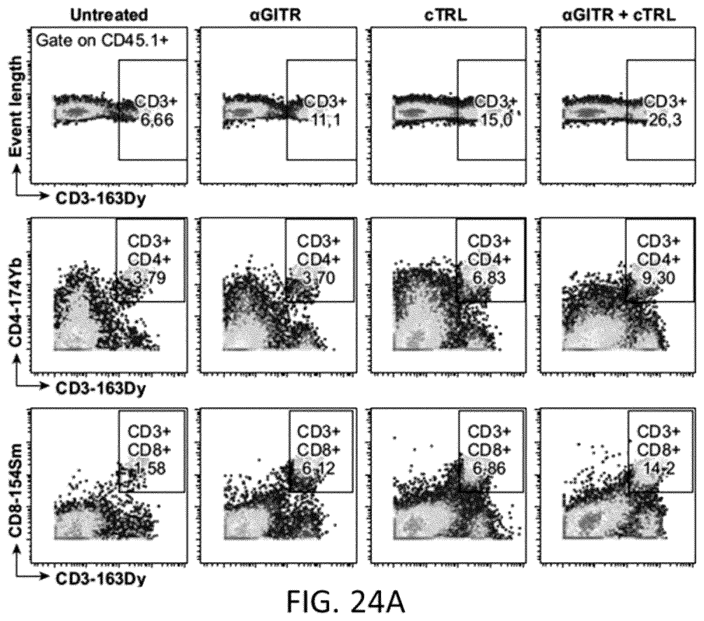

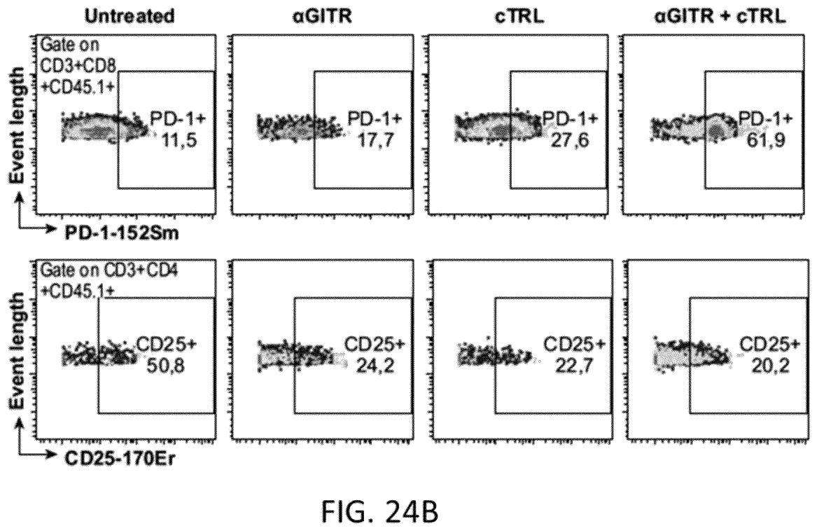

In addition to ICB, we also confirmed that the cocktail of cTRLs and co-stimulatory molecules (e.g., GITR) can yield synergistic effects when treating mouse mesothelioma (FIG. 4E). The combination of anti-GITR antibodies and cTRLs yields 47% extended median survival compared to untreated animals. CyTOF analysis revealed that the administration of cTRLs promoted the infiltration of endogenous CD4 and CD8 cells (FIGS. 4F-G, FIG. 24A), similar to what was observed in the MC-38 model. The cocktail of CTRLs and anti-GITR antibodies upregulated the frequency of CD8+ and CD8+PD-1+ cytotoxic T cells and reduced the number of CD4+CD25+ regulatory T cells (FIG. 24B), generating a more proinflammatory TME for better tumor control. This observation matches with the intrinsic function of Trm cells—that is to secrete proinflammatory cytokines at the diseased site to trigger downstream adaptive immune response.

CD103 Expression Defines cTRLs in Human PBMCs

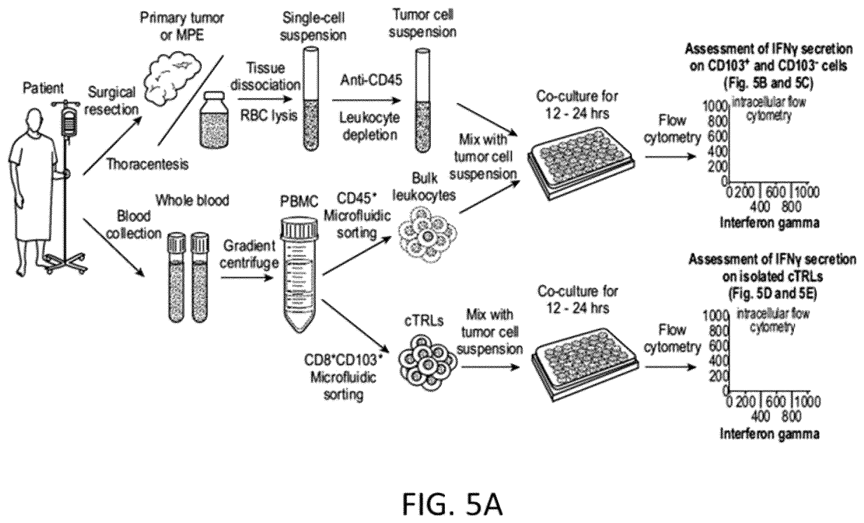

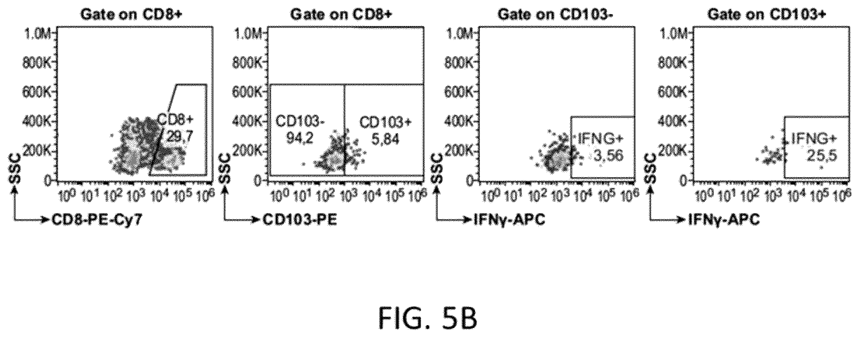

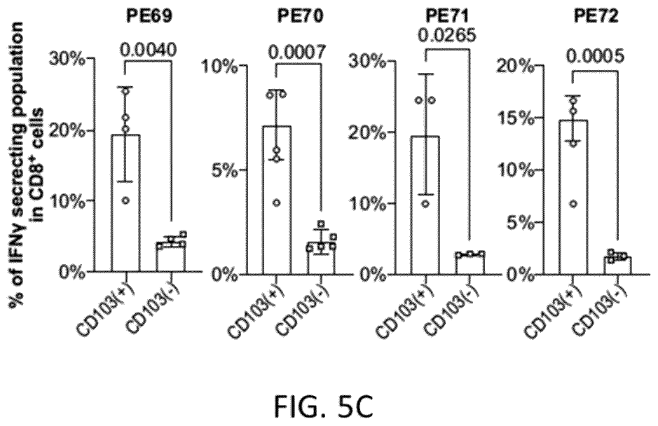

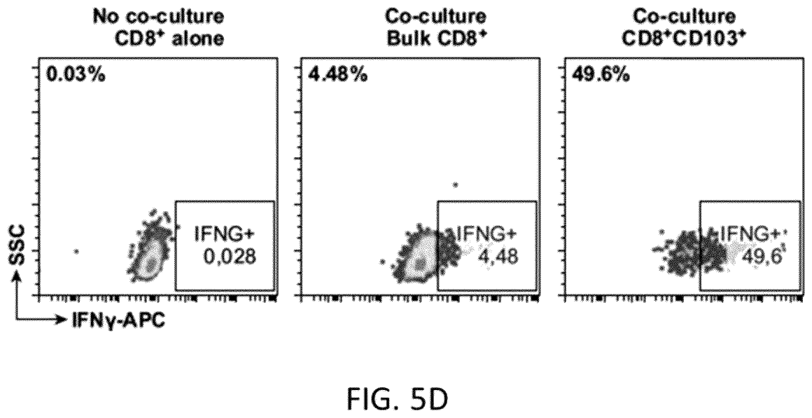

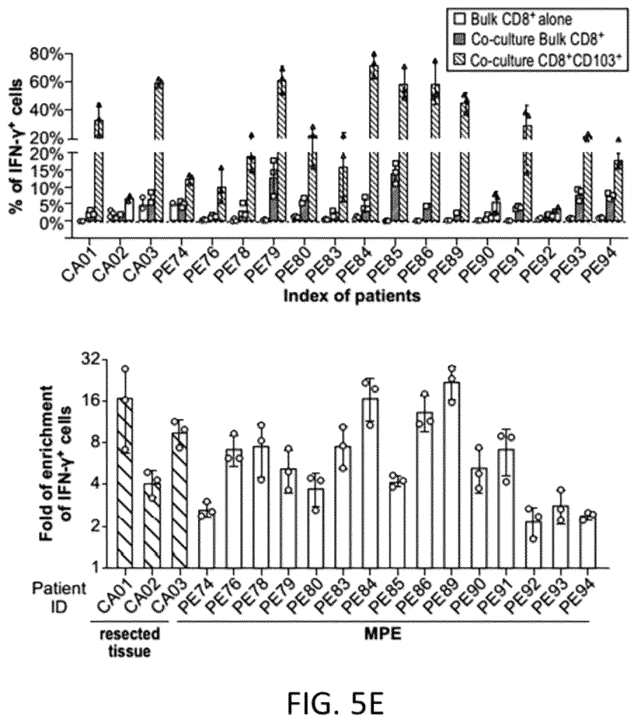

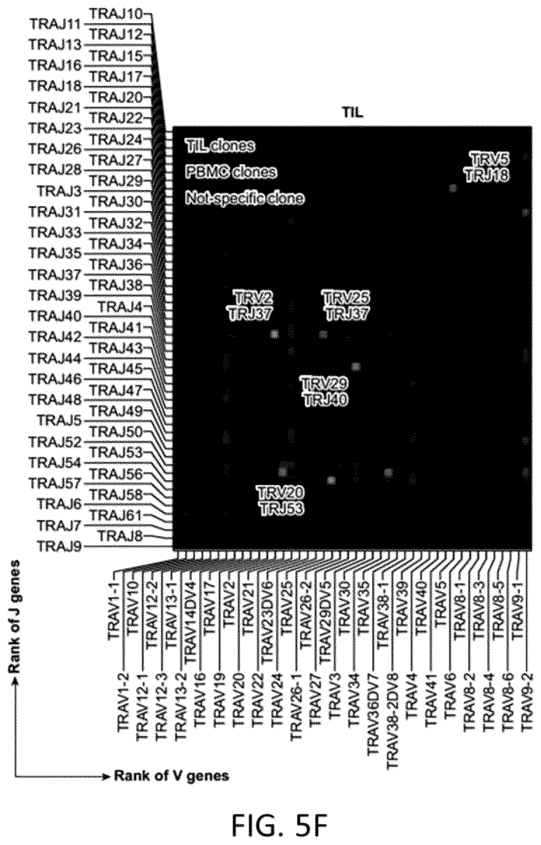

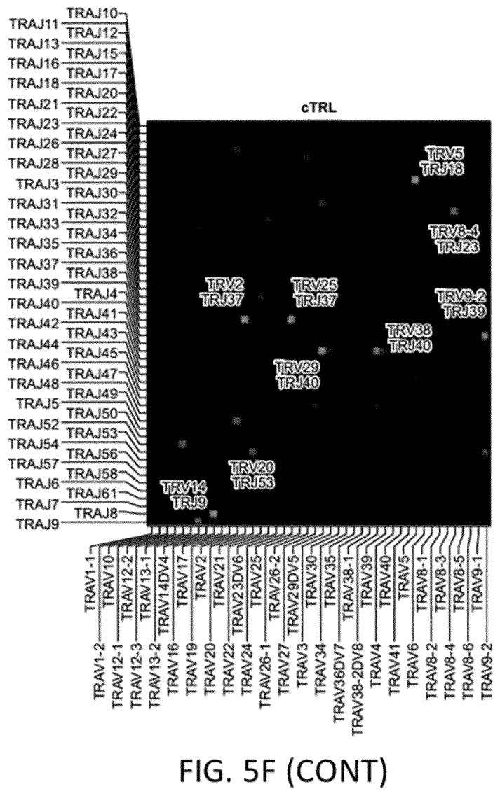

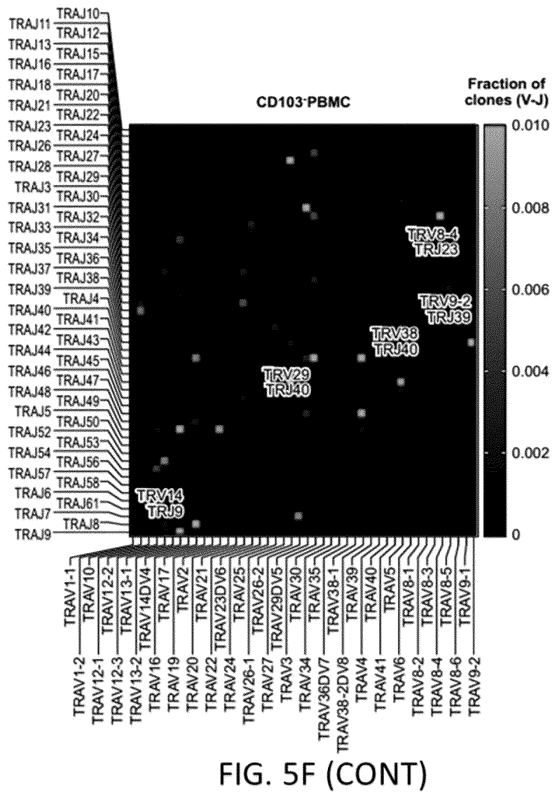

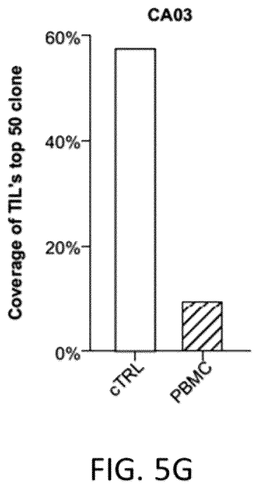

To investigate the presence of cTRLs in human specimens, samples from a small cohort of immunotherapy-naïve patients with a variety of solid tumors (N=22) were acquired and PBMCs were analyzed to determine if CD8+CD103 cells were tumor-reactive. To stimulate the immune cells during co-culture, tumor cells were isolated either from resected tissue or malignant pleural effusions (MPEs). The major tumor types in this study included lung, colon, breast, and mesothelioma. We co-cultured the bulk PBMCs with CD45+ depleted dissociated tumor cells or MPE-derived tumor cells (FIG. 5A). After 12-24 hours, the fraction of IFN-γ secreting cells in CD8+CD103− and CD8+CD103 subpopulations was measured by intracellular flow cytometry (FIGS. 5B and 5C). We found that the majority of IFN-γ secreting cells were among the CD8+CD103 T cell subset. In addition, we isolated CD8+CD103 cells before performing co-culture (FIGS. 5A, 5D-5E). A global increase of IFN-γ secretion after CD103− mediated isolation was consistently observed, suggesting that sorting based on CD8+CD103 helped to define and enrich for a tumor-reactive T cell subpopulation. In addition, we found the presence of cTRL is common in immunotherapy-naïve PBMC—this is in line with the previous study reporting neoantigen-reactive T cells can be detected prior to immunotherapy.

In a subset of patients, we also isolated the TILs, cTRLs, and CD8+CD103− PBMC for TCRseq using a workflow similar to FIG. 1B. Among three sequenced samples, similarity between the major clones presented in TILs and cTRLs was consistently observed (FIGS. 5F-5G). For example, in the case of CA03, cTRLs share 4 major clones with TILs-TRAV2/TRAJ37, TRAV25/TRAJ37, TRAV20/TRAJ53, and TRAV5/TRAJ18 (FIG. 5F). Over 25% of the cTRLs belong to one of these four clonotypes. In contrast, only 0.6% of the CD8+CD103− PBMC fall into these clonotypes. Overall, similar to mouse samples, CD8+CD103 cTRLs cover about 60% of the top 50 clones (ranked by fractions) presented in CD8+ TILs (FIG. 5G). The significant overlap of TCR proves the tumor reactivity of CD8+CD103 cTRLs15 from the perspective of clonal analysis. This is in line with a set of prior studies reporting that the positive expression of CD103 defines the tumor-reactive TILs across human breast, lung, cervical, and oral cancer.

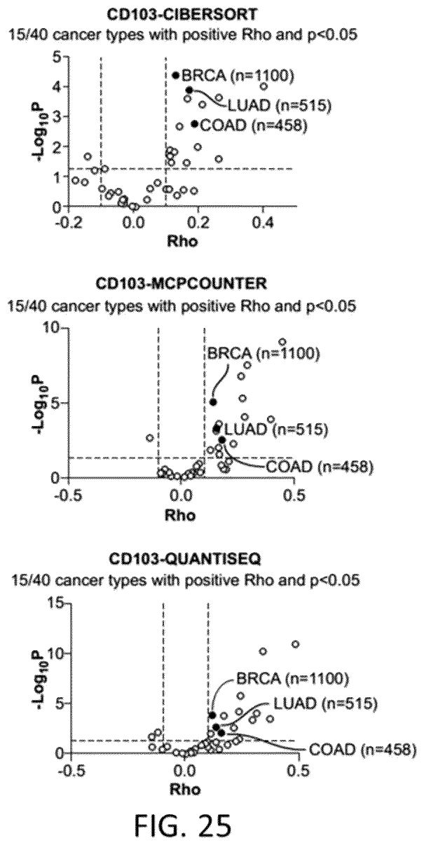

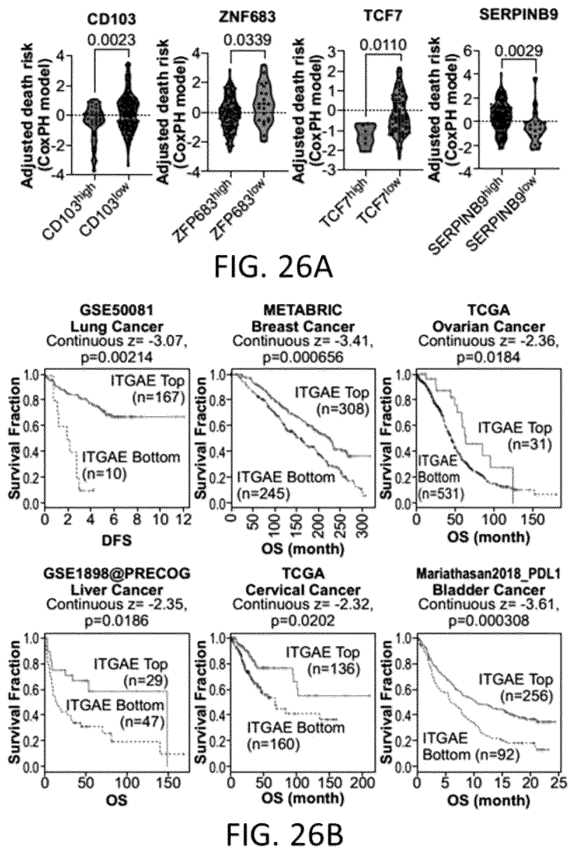

Taken together, our results showcase that the expression of CD103 also defines the CTRL population in human PBMC. Although the therapeutic use of human CD8+CD103 cTRLs is yet to be experimentally examined, bioinformatic analyses have predicted their strong potential. For example, the TIMER (Tumor IMmune Estimation Resource) algorithm reveals that the expression of CD103 has strong correlation with a high level of immune infiltration in many cancer types (FIG. 25) using predicted immune cell fraction from bulk RNAseq. In addition, the TIDE (Tumor Immune Dysfunction and Exclusion) algorithm on the mRNA-seq data across 194 cohorts of solid tumors shows that the upregulated expression of intratumoral ITGAE correlates with statistically lower death risk and good prognosis over multiple cancer types (FIGS. 26A-26B). Furthermore, a meta-analysis involving 2,824 patients reports that patients with intratumoral CD103 immune cells were associated with favorable survivals.

### Conclusions

Thus far, the production of therapeutic TILs requires an accessible tumor lesion for excisional biopsy as source material for TIL isolation and expansion. However, such lesions are not always available from patients and moreover, surgery on patients bearing unresectable cancers can pose a substantial risk. The discovery of cTRLs in blood circulation highlights a new strategy for isolated cells for ACT. The minimal invasiveness of blood collection makes the cTRL acquisition a more feasible and amenable process for the patients compared to TIL therapy. In addition, this analysis reveals that the cTRLs have sufficient coverage of dominant clones in TILs and are primed to target similar tumor microenvironment. These unique characteristics grant their therapeutic potency against metastatic tumors. Hence, the implementation of cTRLs would greatly extend the applicability of adoptive cell therapy and may provide a new treatment option for late-stage patients with unresectable and/or metastasized tumors.

Another outstanding issue for TIL therapy is the low CR rates (<20%) in nonmelanoma cancer in the clinic. At present, it is unclear which phenotypes of TILs should be used and how often should TILs be administrated to deliver a persistent therapeutic outcome54. We noticed that the cTRLs were responsive to immune checkpoint blockade (ICB) and co-stimulatory molecules—the combination of ACT and ICB achieved 80% CR rate in mouse colon cancer models. Considering the ease of collection/administration of cTRLs, the cocktail of ACT and ICB may hold promise as an immune-oncology combination. In addition, a recent phase 1/2 trial indicates that multiple doses of CAR-T therapy can boost the overall response rate against B-cell leukemia⁵⁵. Therefore, the administration of multiple doses of cTRLs may also offer better therapeutic outcomes and it will likely be straightforward to isolate the cTRLs in a repeated fashion.

Taken together, our study provides new evidence supporting the presence of CD8+ tumor-reactive lymphocytes in circulation and highlights the usefulness of such a population for cancer immunotherapy. Future studies shall focus on verifying the therapeutic potency of cTRLs in humanized models. In addition, recent studies have suggested various molecular signatures of potent TIL subpopulations, such as CD39-/CD69-56 and XCL1+57. It may be fruitful to include additional rounds of selection on expanded cTRLs to further boost their therapeutic potency.

### Example 2: Isolation of Tumor-Reactive TRLs in Blood Circulation

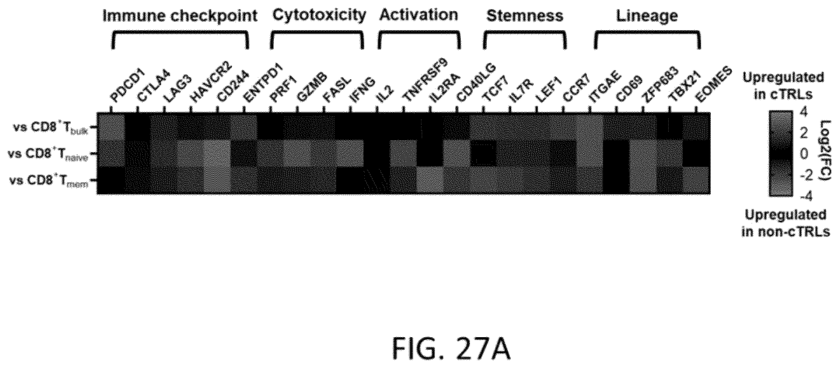

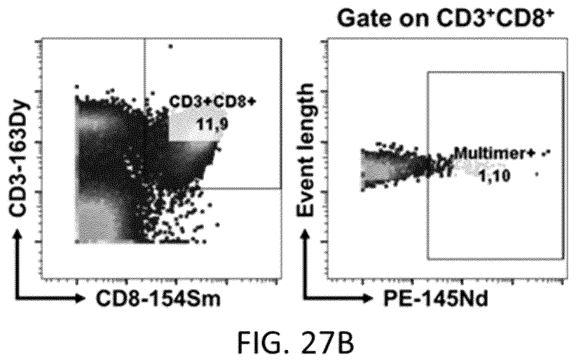

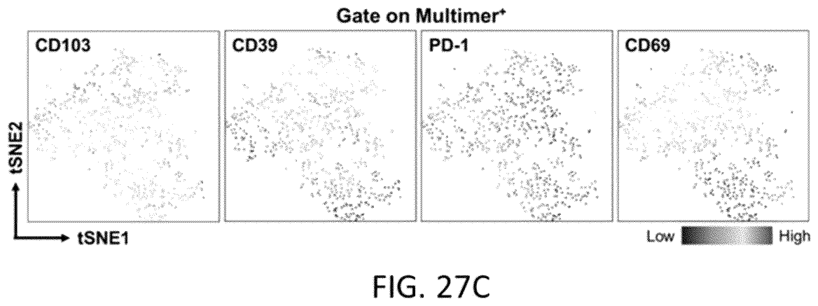

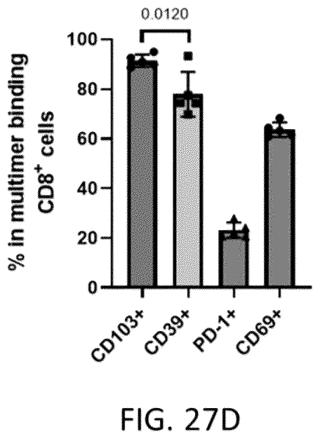

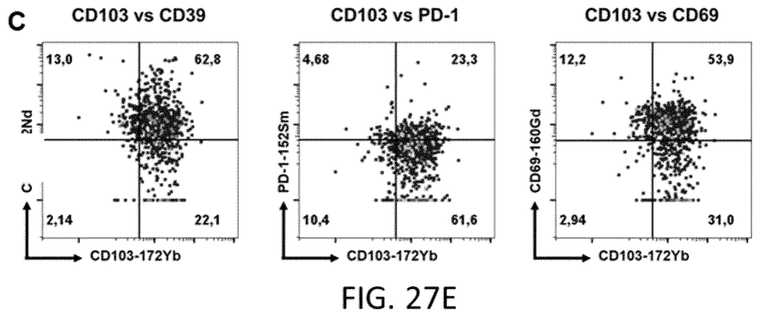

For the OVA-reactive cTRLs isolated from the melanoma model, a tissue-resident memory (Trm) phenotype was detected with strongly upregulated expression of ITGAE compared to non-cTRL bulk CD8+ cells and upregulated expression of ZFP683, a transcriptional hallmark of Trm (FIG. 27A).

Circulating CD8+ lymphocytes were isolated from mice bearing B16F10 and AE17 cancer cell lines expressing the OVA epitope. Flow cytometric analysis indicated that the OVA-reactive cTRLs are almost exclusively CD103+ (FIG. 27B). Collectively, about 30% of the CD8+CD103+ cells in circulation are OVA-reactive (FIG. 27C), which yields up to 50-fold enrichment of OVA-reactive T cells. It suggests CD103, alone or together with other markers, defines T cells with elevated potency for adoptive cell therapy and immune checkpoint blockade.

Further analysis showed that CD39+ lymphocytes, in addition to CD103, were present in the relative cTRLs (FIGS. 27B-27D). As illustrated in FIG. 27D, approximately 80% or more of the CD8+ reactive CTRL population expressed CD103 and/or CD39 markers. Moreover, FIG. 27E demonstrates that about 62.8% of the reactive cTRLs identified were CD39+CD103+ lymphocytes.

### Example 3: T Cell Expansion in Various Culture Conditions in Feeder-Cell Free System

The effects of different culture conditions on T cell expansion in the feeder-cell free system were investigated. Cells were cultured at initial seeding densities of either 1,000 or 10,000 cells per well in 96-well plates. Table 3 presents a summary of the specialized T cell media that were assessed: TexMACS (Miltenyi Biotec), OpTmizer (Thermo Fisher Scientific), and ImmunoCult (STEMCELL Technologies). These media were supplemented with either 10% or lower human AB serum (BioIVT) or serum substitute (Gibco), a concentration of 50 IU/mL or higher of human interleukin 2 (IL-2), and a T cell stimulator (OKT3, T cell TransAct, etc.). The medium was replaced every 2 to 3 days, and cells were passaged and moved to larger wells once the concentration reached 0.1×10\hat{ }5 cells/mL or higher.

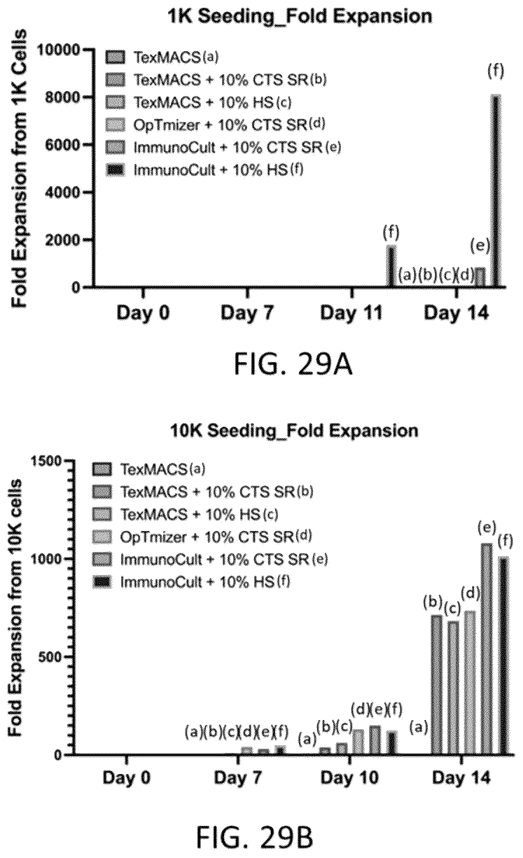

As demonstrated in FIGS. 29A and 29B, higher initial cell concentrations resulted in faster expansion rates and improved viability, while lower concentrations led to slower expansion and reduced viability. By Day 14, the conditioned media comprising of ImmunoCult and 10% CTS SR, seeded at 10,000 cells, resulted in the most significant fold expansion. The conditioned media containing ImmunoCult, and 10% HS seeded at 10,000 cells yielded comparable results.

TABLE 1
T cell conditioned culture media

| | Serum/Serum | | | Initial Cell |
|---|---|---|---|---|
| Media | Replacement | Cytokines | Stim | Seeding |
| Miltenyi | None | 50 IU/ml | 1:100 | 1K and 10K |
| TexMACS | | | Transact | |
| | | | (Miltenyi) | |
| Miltenyi | 10% CTS | 50 IU/ml | 1:100 Transact | 1K and 10K |
| TexMACS | Serum | | (Miltenyi) | |
| | Replacement | | | |
| | (Gibco) | | | |
| Miltenyi | 10% Human | 50 IU/ml | 1:100 Transact | 1K and 10K |
| TexMACS | Serum | | (Miltenyi) | |
| | Replacement | | | |
| | (BioIVT) | | | |
| Gibco | 10% CTS | 50 IU/ml | 1:100 Transact | 1K and 10K |
| OpTmizer | Serum | | (Miltenyi) | |
| | Replacement | | | |
| | (Gibco) | | | |
| Stemcell | 10% CTS | 50 IU/ml | 1:100 Transact | 1K and 10K |
| Immunocult | Serum | | (Miltenyi) | |
| | Replacement | | | |
| | (Gibco) | | | |
| Stemcell | 10% CTS | 50 IU/ml | 1:100 Transact | 1K and 10K |
| Immunocult | Serum | | (Miltenyi) | |
| | Replacement | | | |
| | (Gibco) | | | |

### Example 4: Isolation of Human Tumor-Reactive Lymphocytes

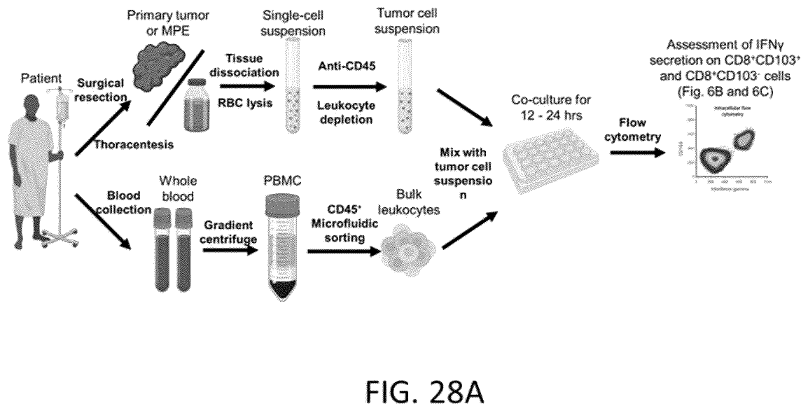

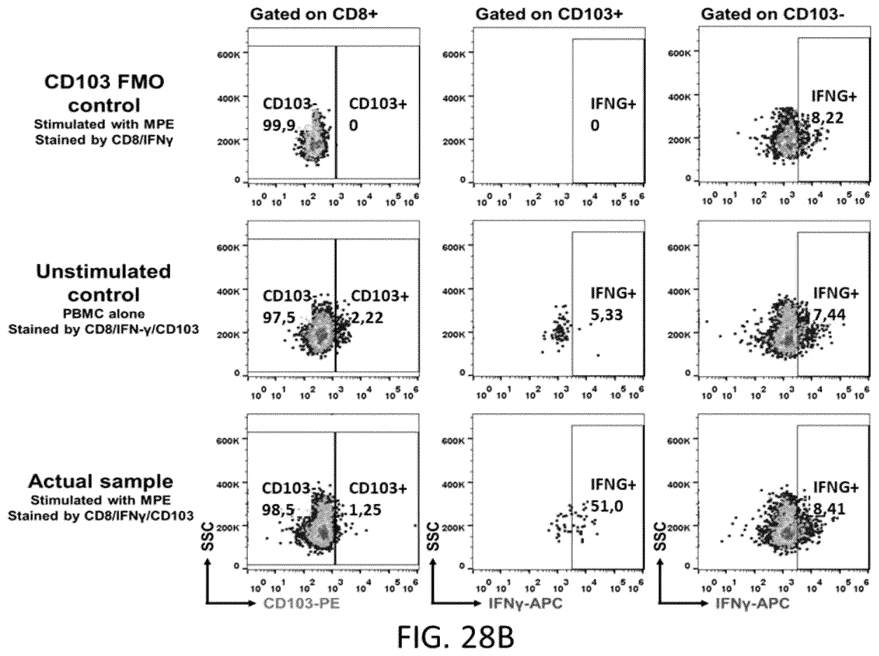

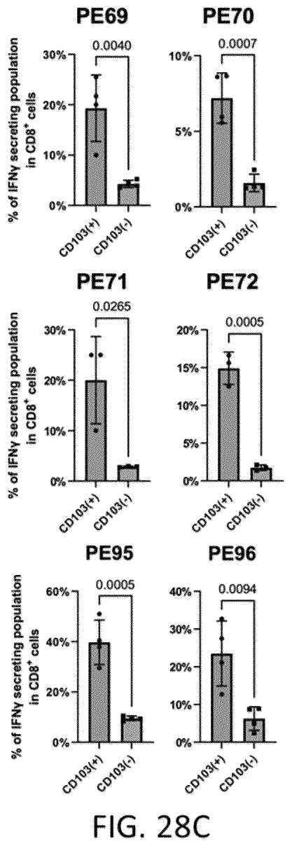

The presence of cTRLs in human specimens was investigated by analyzing paired malignant pleural effusion (MPE) samples and PBMCs from 6 immunotherapy-naïve patients' samples. The bulk PBMCs were co-cultured with CD45+ depleted MPE-derived tumor cells and measured the fraction of IFN-γ secreting cells in CD8+CD103− and CD8+CD103+ subpopulations by intracellular flow cytometry post 12-24 hours (FIG. 28A). The panel for flow cytometry included CD103 FMO (fluorescence minus one) and unstimulated (PBMC alone) controls for the precise identification of CD103+ populations (FIG. 28B). A small portion of interferon gamma (IFNγ) secreting cells were observed in the unstimulated control (5.3% for CD103+, 7.4% for CD103−). When stimulated by MPE-derived tumor cells, a significant portion of CD103+ T cells exhibited IFNγ secretion (51%) while the percentage of IFNγ secreting CD103− T cells remained roughly unchanged (8.4%) (FIG. 28C). This observation suggested that the IFNγ secretion in CD103+ T cells is tumor-dependent.

Table 2 demonstrates the results of isolated CD103+ circulating TRLs from patient samples using the methods and device (e.g., microfluidic device) described herein. Table 2 presents a comparison of the post-isolation purity of CD103+ cells with the initial purity, showing a significant increase in purity (average % purity of 0.648 vs. 21.26, respectively). Additionally, the methods and device described herein resulted in an enhanced fold enrichment and % recovery of CD103+ cells from patient samples.

TABLE 2
Microfluidic device isolation of CD103+
circulating TRLs of the patient samples

| | | Post- | | |
|---|---|---|---|---|
| | Initial CTRL | Isolation | | |
| | Purity (%, | Purity (%, | Fold | % Recovered |
| Patient | CD103+) | CD103+) | enrichment | CD103+ cells |
| 888976935 | 1.2 | 36.2 | 11.6 | 39.6 |
| P803 | 0.42 | 11.9 | 28.3 | 42.4 |
| P518 | 0.76 | 28.1 | 37.0 | 39.5 |
| P468 | 0.33 | 11.6 | 27.6 | 36.2 |
| P6389 | 0.53 | 18.5 | 34.9 | 38.9 |

### Example 5: Tumor-Reactive Lymphocytes Expression Profile

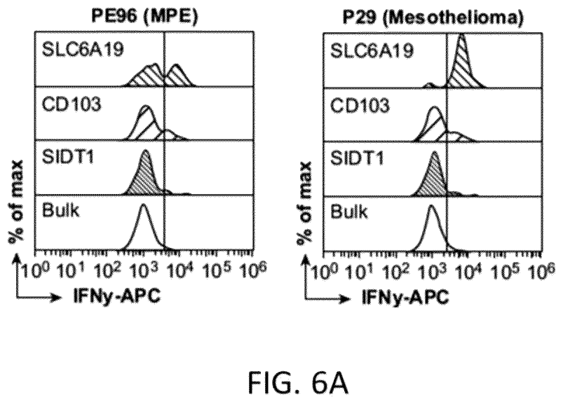

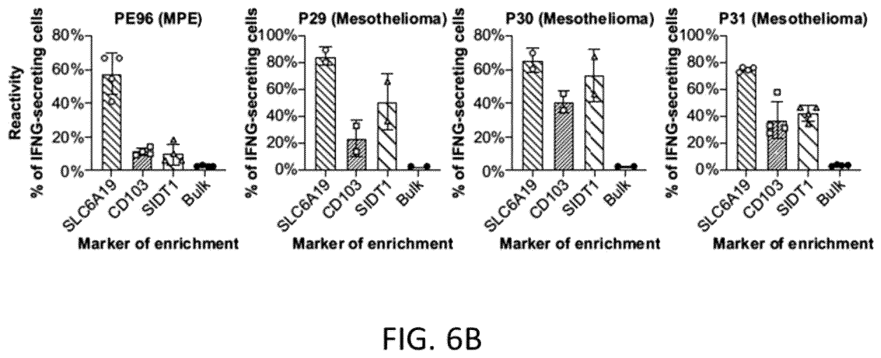

The goodness of other hits obtained from the RNAseq of B16/CT26 models, including SLC6A19 and SIDT1 was quantified (FIGS. 6A and 6B). As shown in FIG. 6A, it was found that the SLC6A19 or SIDT1-expressing cells in CD8+ populations also help to improve tumor reactivity in a cohort of 4 patients with lung cancer/mesothelioma. Indeed, as shown in FIG. 6B, SLC6A19 is the most specific marker that defines the CTRL population in PBMC. 60-80% of SLC6A19+CD8+ cells secrete IFNγ when co-culturing with corresponding tumor cells or malignant pleural effusion. Taken together, the rightness of SLC6A19, CD103, and SIDT1 as the isolation marker for circulating TRL (CTRL) was validated.

### Example 6: Comparison of FACS/MACS/Microfluidic for CD103 Lymphocytes Sorting

Three cell separation techniques: Fluorescence-Activated Cell Sorting (FACS), Magnetic-Activated Cell Sorting (MACS), and Microfluidic sorting were examined using device targeting CD8+CD103+ markers for Tumor-Resident Lymphocytes (TRLs) isolated from the E0771 mouse breast cancer model.

Table 3 illustrates that Microfluidic sorting (36.3±8.0%) yielded purity levels comparable to FACS (43.1±16.7%). Additionally, Microfluidic sorting demonstrated the highest cell recovery rate (73.2±10.2%), significantly surpassing both FACS (4.5±1.6%) and MACS (30.1±10.0%). The enrichment ratio was the highest for Microfluidic sorting (69.4), followed by MACS (6.3), and was the lowest for FACS (5.0). Microfluidic sorting displayed the highest throughput per device (200×10\hat{ }6 cells/hr.) and per setup (2000×10\hat{ }6 cells/hr.), while MACS followed with 50×10\hat{ }6 cells/hr. per device and 200×10\hat{ }6 cells/hr. per setup. FACS had the shortest total assay time of 30 minutes; however, both MACS and Microfluidic sorting exhibited comparable assay times of 20-40 minutes per marker. Although FACS had a shorter assay time, it is crucial to consider its lower recovery rate and throughput in comparison to the other methods. These results demonstrated that Microfluidic sorting provides the highest recovery rate, enrichment ratio, and throughput, making it a promising method for cell separation applications.

TABLE 3
TRL separation by FACS, MACS, and Microfluidic

| | FACS | MACS | Microfluidic |
|---|---|---|---|
| Purity | 43.1 ± 16.7% | 8.1 ± 3.6% | 36.3 ± 8.0% |
| Recovery | 4.5 ± 1.6% | 30.1 ± 10.0% | 73.2 ± 10.2% |
| Enrichment | 5.0 | 6.3 | 69.4 |
| Ratio | | | |
| Throughput | 2 × 10⁶ | 50 × 10⁶ cells/hr. | 200 × 10⁶ cells/hr. |
| per device | cells/hr. | | |
| Throughput | 2 × 10⁶ | 200 × 10⁶ cells/hr. | 2000 × 10⁶ cells/hr. |
| per setup | cells/hr. | | |
| Assay time | 30 min in total | 20-40 min per | 20-40 min per |
| | | marker | marker |

### Example 7: Cell Preparation for CAR-T or TCR-T Engineering

Peripheral blood samples are collected from cancer patients. Circulating tumor-reactive immune cells (e.g., cTRLs) expressing CD103, CD36, SLC6A19 and/or SIDT1 are isolated using Microfluidic sorting as described herein. These isolated immune cells are then genetically enhanced and modified to express CARs targeting various tumor antigens (e.g, CD19). In some cases, CAR-T cell therapy can be further modified to reduce the risk of graft-versus-host disease (GVHD) by removing the T cell receptor (e.g., endogenous TCR) or the alpha-beta (αβ) TCR chains. The modified cells are expanded in vitro to generate a sufficient number of CAR-T cells for infusion.

The personalized CAR-T cell therapy is administered using various dosing schemes, including a single-dose administration (e.g., patients receive a single infusion of CAR-T cells), a dose-escalation approach (e.g., patients receive gradually increasing doses of CAR-T cells over multiple infusions) or a combination therapy (e.g., patients receive CAR-T cells in combination with other immunotherapies, chemotherapies, or targeted therapies). The number of CAR-T cells (e.g., dose) infused into a patient can vary depending on factors such as the specific CAR-T cell product, the patient's body weight, disease type, disease burden, and the severity of the patient's condition. For example, a pediatric and young adult patient can receive a single dose of 0.2 to 5.0×10\hat{ }6 CAR-positive viable T cells per kg of body weight, and an adult patient can receive a single dose of 0.6 to 6.0×10\hat{ }8 CAR-positive viable T cells or A single dose of 2.0×10\hat{ }6 CAR-positive viable T cells per kg of body weight.

The response to treatment is monitored using clinical and imaging evaluations, including periodic blood tests to assess levels of circulating tumor-reactive cells, tumor imaging using techniques such as CT, MRI, or PET scans to evaluate changes in tumor size and location, and immune monitoring to assess the persistence and expansion of infused CAR-T cells and evaluate changes in immune cell populations.

While preferred embodiments of the present inventive concepts have been shown and described herein, it will be obvious to those skilled in the art that such embodiments are provided by way of example only. Numerous variations, changes, and substitutions will now occur to those skilled in the art without departing from the inventive concepts. It should be understood that various alternatives to the embodiments of the inventive concepts described herein may be employed in practicing the inventive concepts. It is intended that the following claims define the scope of the inventive concepts and that methods and structures within the scope of these claims and their equivalents be covered thereby.

1. (canceled)

2. A system, comprising:
a fluid sample obtained from a subject, wherein the fluid sample comprises a population of cells comprising CD103+ lymphocytes, and
a microfluidic device comprising a magnetic capture zone disposed in a channel of the microfluidic device,
wherein the microfluidic device is configured to magnetically separate the population of cells from the fluid sample.

3. The system of claim 2, wherein the CD103+ lymphocytes are CD8+CD103+ lymphocytes.

4. The system of claim 2, wherein the CD103+ lymphocytes are CD8+CD39+CD103+ lymphocytes.

5. The system of claim 2, wherein the fluid sample is obtained from a peripheral blood sample or a processed sample obtained therefrom.

6. The system of claim 2, wherein the fluid sample comprises a pleural effusion.

7. The system of claim 2, wherein the fluid sample comprises ascites.

8. The system of claim 2, wherein the system further comprises an antibody or antigen binding fragment thereof capable of binding a TRL surface protein.

9. The system of claim 8, wherein the TRL surface protein is CD8 or CD103.

10. The system of claim 8, wherein the antibody or antigen-binding fragment is conjugated to a magnetic nanoparticle.

11. The system of claim 2, wherein the system further comprises a plurality of major histocompatibility complex (MHC) multimers mimicking a tumor epitope.

12. The system of claim 11, wherein at least one of the plurality of MHC multimers is conjugated to a linker molecule comprising a fluorophore or a magnetic particle.

13. The system of claim 2, wherein the microfluidic device comprises a plurality of magnetic capture zones, wherein the plurality of magnetic capture zones are disposed to spatially separate cells with different degrees of magnetization.

14. The system of claim 2, wherein the system further comprises a cell culture configured to culture therapeutically enhanced cells derived from the population of the CD103+ lymphocytes.

15. The system of claim 2, wherein the system further comprises a pharmaceutically acceptable: excipient, diluent, or vehicle, wherein the population of the CD103+ lymphocytes is formulated with the pharmaceutically acceptable: excipient, diluent, or vehicle for administration to a subject having or suspected of having cancer.

16. A system, comprising:
a fluid sample obtained from a subject, wherein the fluid sample comprises a population of cells comprising CD39+ lymphocytes, and
a microfluidic device comprising a magnetic capture zone disposed in a channel of the microfluidic device,
wherein the microfluidic device is configured to magnetically separate the population of cells from the fluid sample.

17. The system of claim 16, wherein the system further comprises an antibody or antigen binding fragment thereof capable of binding a TRL surface protein.

18. The system of claim 17, wherein the antibody or antigen-binding fragment is conjugated to a magnetic nanoparticle.

19. A system, comprising:
a fluid sample obtained from a subject, wherein the fluid sample comprises a population of cells comprising SIDT1+ lymphocytes, SLC6A19+ lymphocytes, or a combination thereof, and
a microfluidic device comprising a magnetic capture zone disposed in a channel of the microfluidic device,
wherein the microfluidic device is configured to magnetically separate the population of cells from the fluid sample.

20. The system of claim 19, wherein the system further comprises an antibody or antigen binding fragment thereof capable of binding a TRL surface protein.

21. The system of claim 20, wherein the antibody or antigen-binding fragment is conjugated to a magnetic nanoparticle.
